(19) United States
(12) Patent Application Publication
(10) Pub. No.: US 2023/0218631 A1
(43) Pub. Date: Jul. 13, 2023

(54) G9A INHIBITION DECREASES STRESS-INDUCED AND DEPENDENCE-INDUCED ESCALATION OF ALCOHOL DRINKING

(71) Applicant: MUSC Foundation for Research Development, Charleston, SC (US)

(72) Inventors: Ethan Anderson, North Charleston, SC (US); Christopher Cowan, Mount Pleasant, SC (US)

(21) Appl. No.: 18/155,587

(22) Filed: Jan. 17, 2023

Related U.S. Application Data
(63) Continuation-in-part of application No. PCT/US2021/042044, filed on Jul. 16, 2021.
(60) Provisional application No. 63/052,750, filed on Jul. 16, 2020.

(51) Int. Cl. A61K 31/551; A61K 31/517; A61K 31/47; A61K 31/404; A61P 25/32

(52) U.S. Cl. CPC A61K 31/551; A61K 31/517; A61K 31/47; A61K 31/404; A61P 25/32

(57) ABSTRACT

Provided are methods for reducing substance consumption by subjects. In some embodiments, the presently disclosed methods include administering to a subject in need thereof a composition that includes an effective amount of an inhibitor of an EHMT2/G9A biological activity. In some embodiments, the inhibitor of an EHMT2/G9A biological activity is a small molecule inhibitor, a nucleic acid-based inhibitor, and anti-EHMT2/G9A antibody or a fragment or derivative thereof, or any combination thereof. Also provided are methods for reducing relapse vulnerability in subjects that have Alcohol Use Disorder (AUD) and/or another substance use disorder. In some embodiments, the presently disclosed methods further include administering at least one additional therapy to subjects, including but not limited to behavioral therapies such as cognitive behavioral therapies.

### CROSS REFERENCE TO RELATED APPLICATION

This application is a continuation-in-part of PCT International Patent Application Serial No. PCT/US2021/042044, filed Jul. 16, 2021, herein incorporated by reference in its entirety, which claims priority to and benefit of U.S. Provisional Patent application Ser. No. 63/052,750, filed Jul. 16, 2020, the disclosure of which is incorporated herein by reference in its entirety.

### GRANT STATEMENT

This invention was made with government support under DA027664, DA046513, AA10761, and DA032708 awarded by the National Institutes of Health. The government has certain rights in the invention.

### REFERENCE TO SEQUENCE LISTING SUBMITTED ELECTRONICALLY

The content of the electronically submitted sequence listing in ASCII text file (Name: 1586_21_2_PCT_ST26.XML; Size: 106,034 bytes; and Date of Creation: Jan. 15, 2023) filed with the application via the Patent Center is incorporated herein by reference in its entirety.

### TECHNICAL FIELD

The presently disclosed subject matter relates in some embodiments to compositions and methods for treating stress-induced and/or dependence-induced escalation of alcohol drinking and/or other substance use disorders.

### BACKGROUND

Alcohol use disorder (AUD) is a chronic, relapsing disease that is difficult to treat due in part to co-morbidities with other neuropsychiatric illnesses like stress- or anxiety-related disorders. In addition, evidence suggests that the chronic use of abused substances, like alcohol, can lead to the formation of lasting stress disorders produced by dysregulation of stress-response systems in the brain (Becker, 2012). However, the mechanisms that lead to these stable changes are currently unknown. A second reason for the pervasiveness of AUD is that heavy alcohol drinking can produce alcohol dependence, and alcohol dependence further dysregulates the body's stress systems (Becker, 2012) to increase alcohol drinking. Therefore, targeting dependence- and/or stress-related alcohol drinking clinically could greatly reduce “heavy drinking” in AUD patients, potentially halt the downward spiral of “the dark side of addiction”, and reduce stress-related relapse in abstinent patients. Since many AUD patients present with co-morbid psychiatric diseases and/or disorders related to stress (Moss et al, 2010), targeting stress-related alcohol drinking could be particularly useful.

Epigenetics, which involves long-lasting changes in chromatin landscape and gene expression, has emerged as a likely mechanism underlying the enduring changes in brain functions that contribute to the myriad symptoms of alcohol use disorder (AUD) and substance use disorder (SUD), including the sensitivity to triggers of relapse, such as stress. Several epigenetic enzymes, such as histone deacetylases and histone methyl transferases, are regulated by acute or chronic exposure to abused substances and can influence the development of AUD/SUD-related behaviors (Anderson et al., 2018a).

One such enzyme, G9A (also known as euchromatic histone-lysine N-methyltransferase 2 or EHMT2), is a histone methyltransferase that catalyzes di-methylation on lysine 9 of histone H3 (H3K9me2; Maze et al., 2010; Covington et al., 2011). H3K9me2 is typically associated with condensed chromatin and repression of target gene expression; and G9A is a major regulator of this histone mark in NAc neurons (Anderson et al., 2018a). Interestingly, both cocaine and opioids regulate G9A levels in the NAc (Maze et al., 2010; Sun et al., 2012), and in cocaine self-administration assays, G9A has bi-directional effects on motivation to take cocaine and stress-induced reinstatement of cocaine seeking—a model of relapse-like behavior in rodents (Anderson et al., 2018b; Anderson et al., 2019). In addition, G9A in the NAc has bidirectional effects on anxiety-like behaviors (Anderson et al., 2018b; Anderson et al., 2019). Similar to cocaine and opioids, G9A is regulated by alcohol exposure in the developing brain in models of fetal alcohol syndrome, in the amygdala in adult mice, and it is required for alcohol-induced changes in H3K9me2 levels in in vitro models (Qiang et al., 2011; Subbanna et al., 2013; Subbanna and Basavarajappa, 2014; Subbanna et al., 2014; Gangisetty et al., 2015; Veazey et al., 2015; Berkel et al., 2019); however, studies of G9A's potential role in the NAc as it relates to AUD-associated behavior is unexplored.

As set forth herein, the role and regulation of NAc G9A were tested in an animal model of alcohol use disorder (AUD). It is demonstrated here that CIE exposure-induced ethanol dependence in mice reduced both G9A and H3K9me2 levels in the adult NAc, but not in dorsal striatum, and that G9A in NAc was required for stress-regulated changes in alcohol drinking. Moreover, chronic systemic administration of a G9A inhibitor (2-(4,4-difluoropiperidin-1-yl)-N-(1-isopropylpiperidin-4-yl)-6-methoxy-7-(3-(pyrrolidin-1-yl)propoxy)quinazolin-4-amine, also called UNC0642; Chemical Abstracts Service Registry (CAS) No.: 1481677-78-4) blocked both stress-potentiated and dependence-induced ethanol drinking in male mice. These findings suggested that chronic ethanol use, similar to other abused substances, produced a reduction of G9A in the NAc, and that this reduction limited stress-induced and dependence-induced changes in ethanol drinking.

Furthermore, systemic inhibition of G9A activity reduced stress-potentiated and dependence-induced ethanol drinking, suggesting a novel therapeutic approach to reduce stress-induced and dependence-induced heavy drinking in individuals suffering from AUD.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter relates to methods for reducing substance consumption by subjects, such as a subject with a substance use disorder (SUD), optionally alcohol use disorder (AUD). In some embodiments, the methods comprise, consist essentially of, or consist of administering to a subject in need thereof a composition comprising, consisting essentially of, or consisting of an effective amount of an inhibitor of a euchromatic histone-lysine N-methyltransferase 2 (EHMT2/G9A) biological activity, whereby consumption of the substance by the subject is reduced as compared to what would have occurred had the subject not been administered the composition. In some embodiments, the substance is alcohol. In some embodiments, the consumption of alcohol is stress-induced consumption, dependence-induced consumption, or both. In some embodiments, the consumption of alcohol is associated with a kappa opioid receptor (KOR) biological activity in the subject, optionally wherein the KOR biological activity is associated with stress in the subject. In some embodiments, the subject is a human. In some embodiments, EHMT2/G9A inhibitor is selected from the group comprising (2-(4,4-difluoropiperidin-1-yl)-N-(1-isopropylpiperidin-4-yl)-6-methoxy-7-(3-(pyrrolidin-1-yl)propoxy)quinazolin-4-amine, 2-(hexahydro-4-methyl-1H-1,4-diazepin-1-yl)-6,7-dimethoxy-N-(1-(phenylmethyl)-4-piperidinyl)-4-quinazolinamine (also known as Histone Lysine Methyltransferase Inhibitor (CAS 935693-62-2) or BIX 01294 trihydrochloride hydrate), 6-Methoxy-2-morpholin-4-yl-N-(1-propan-2-ylpiperidin-4-yl)-7-(3-pyrrolidin-1-ylpropoxy)quinazolin-4-amine (also known as UNC1479), 6-Chloro-N-(4-ethoxyphenyl)-2-methylquinolin-4-amine (also known as CSV0C018875), CPUY074020 (CAS No. 902279-44-1), 2-(benzoylamino)-1-(3-phenylpropyl)-1H-benzimidazole-5-carboxylic acid, methyl ester (also known as BRD4770, CAS No. 1374601-40-7), Chaetocin (CAS No. 28097-03-2), A-366 (CAS No. 1527503-11-2), a derivative thereof, a metabolic precursor thereof, a metabolic product thereof, a salt thereof, or any combination thereof; and/or is a nucleic acid that binds to and inhibits the activity of an EHMT2/G9A gene product; and/or is an antibody and/or a paratope-containing fragment thereof that binds to and inhibits the activity of an EHMT2/G9A gene product. In some embodiments, the EHMT2/G9A inhibitor is (2-(4,4-difluoropiperidin-1-yl)-N-(1-isopropylpiperidin-4-yl)-6-methoxy-7-(3-(pyrrolidin-1-yl)propoxy)quinazolin-4-amine (UNC0642). In some embodiments, the EHMT2/G9A inhibitor is 6-Methoxy-2-morpholin-4-yl-N-(1-propan-2-ylpiperidin-4-yl)-7-(3-pyrrolidin-1-ylpropoxy)quinazolin-4-amine (UNC1479). In some embodiments, the administering results in a reduction of dimethylation of lysine 9 of histone H3 (H3K9me2) in the subject, optionally a reduction of dimethylation of lysine 9 of histone H3 (H3K9me2) in the nucleus accumbens (NAc) in the subject. In some embodiments, the administering is repeated one or more times a day for at least 1, 2, 3, 4, 5, 6, 7, 10, or 15 days.

In some embodiments, the subject has a stress-related and/or anxiety-related disorder and/or a disorder exacerbated by stress and/or anxiety, optionally wherein the stress-related and/or anxiety-related disorder and/or a disorder exacerbated by stress and/or anxiety is selected from the group consisting of post-traumatic stress disorder (PTSD), panic disorder, social anxiety disorder, general anxiety disorder, and major depressive disorder.

The presently disclosed subject matter also relates in some embodiments to methods for reducing relapse vulnerability in subjects that have a substance use disorder (SUD), optionally Alcohol Use Disorder (AUD). In some embodiments, the methods comprise, consist essentially of, or consist of administering to a subject that has a substance use disorder (SUD), optionally Alcohol Use Disorder (AUD) a composition comprising, consisting essentially of, or consisting of an effective amount of an inhibitor of a euchromatic histone-lysine N-methyltransferase 2 (EHMT2/G9A) biological activity, whereby the effective amount is sufficient to reduce the incidence of stress-related alcohol consumption, dependence-related alcohol consumption, and/or another substance consumption by the subject as compared to what would have occurred had the subject not been administered the composition. In some embodiments, the subject has stress-related alcohol consumption, dependence-related alcohol consumption, or both. In some embodiments, the stress-related alcohol consumption, dependence-related alcohol consumption, or both is associated with a kappa opioid receptor (KOR) biological activity in the subject, optionally wherein the KOR biological activity is associated with stress in the subject. In some embodiments, the subject is a human. In some embodiments, the EHMT2/G9A inhibitor is selected from the group comprising (2-(4,4-difluoropiperidin-1-yl)-N-(1-isopropylpiperidin-4-yl)-6-methoxy-7-(3-(pyrrolidin-1-yl)propoxy)quinazolin-4-amine, 2-(Hexahydro-4-methyl-1H-1,4-diazepin-1-yl)-6,7-dimethoxy-N-(1-(phenylmethyl)-4-piperidinyl)-4-quinazolinamine (also known as Histone Lysine Methyltransferase Inhibitor (CAS 935693-62-2) or BIX 01294 trihydrochloride hydrate), 6-Methoxy-2-morpholin-4-yl-N-(1-propan-2-ylpiperidin-4-yl)-7-(3-pyrrolidin-1-ylpropoxy)quinazolin-4-amine (also known as UNC1479), 6-Chloro-N-(4-ethoxyphenyl)-2-methylquinolin-4-amine (also known as CSV0C018875), CPUY074020 (CAS No. 902279-44-1), 2-(benzoylamino)-1-(3-phenylpropyl)-1H-benzimidazole-5-carboxylic acid, methyl ester (also known as BRD4770, CAS No. 1374601-40-7), Chaetocin (CAS No. 28097-03-2), A-366 (CAS No. 1527503-11-2), a derivative thereof, a metabolic precursor thereof, a metabolic product thereof, a salt thereof, or any combination thereof; and/or is a nucleic acid that binds to and inhibits the activity of an EHMT2/G9A gene product; and/or is an antibody and/or a paratope-containing fragment thereof that binds to and inhibits the activity of an EHMT2/G9A gene product. In some embodiments, the EHMT2/G9A inhibitor is (2-(4,4-difluoropiperidin-1-yl)-N-(1-isopropylpiperidin-4-yl)-6-methoxy-7-(3-(pyrrolidin-1-yl)propoxy)quinazolin-4-amine (UNC0642). In some embodiments, the EHMT2/G9A inhibitor is 6-Methoxy-2-morpholin-4-yl-N-(1-propan-2-ylpiperidin-4-yl)-7-(3-pyrrolidin-1-ylpropoxy)quinazolin-4-amine (UNC1479). In some embodiments, the administering results in a reduction of dimethylation of lysine 9 of histone H3 (H3K9me2) in the subject, optionally a reduction of dimethylation of lysine 9 of histone H3 (H3K9me2) in the nucleus accumbens (NAc) in the subject. In some embodiments, the administering is repeated one or more times a day for at least 1, 2, 3, 4, 5, 6, 7, 10, or 15 days.

In some embodiments, the presently disclosed methods further comprise, consist essentially of, or consist of administering at least one additional therapy to the subject. In some embodiments, the at least one additional therapy comprises, consists essentially of, or consists of a behavioral therapy. In some embodiments, the at least one additional therapy comprises, consists essentially of, or consists of a cognitive behavioral therapy.

In some embodiments, the subject has a stress-related and/or anxiety-related disorder and/or a disorder exacerbated by stress and/or anxiety, optionally wherein the stress-related and/or anxiety-related disorder and/or a disorder exacerbated by stress and/or anxiety is selected from the group consisting of post-traumatic stress disorder (PTSD), panic disorder, social anxiety disorder, general anxiety disorder, and major depressive disorder Thus, it is an object of the presently disclosed subject matter to provide compositions and methods for treating stress-induced and dependence-induced escalation of alcohol drinking and/or for treating other substance use disorders.

An object of the presently disclosed subject matter having been stated hereinabove, and which is achieved in whole or in part by the presently disclosed subject matter, other objects will become evident as the description proceeds when taken in connection with the accompanying Detailed Description, EXAMPLES, and Figures as best described herein below.

### BRIEF DESCRIPTION OF THE FIGURES

FIGS. 1A-1F. Ethanol regulates G9A in the accumbens. FIG. 1A is a timeline for CIE vs Air exposure before tissue harvesting. Nucleus accumbens (NAc; FIG. 1B) and dorsal striatum (DStr; FIG. 1C) western blot results for G9A, H3K9me2, H3 total, and B-tubulin following 4 weeks of CIE or Air exposure. FIG. 1D is a timeline for microarray testing following CIE vs Air exposure. FIG. 1E is a representative scatterplot for Air-treated mice following test 3. FIG. 1F is a representative scatterplot CIE-treated mice following test 3. Data are expressed as mean+/−s.e.m. *p<0.05 compared with controls. In FIG. 1D, light gray boxes correspond to 2-bottle choice session timepoints, dark gray boxes correspond to timepoints of No Testing, and the black box corresponds to the timepoint of tissue collection.

FIGS. 2A-2F. Viral-mediated G9A knockdown in the mouse nucleus accumbens has no effect on CIE-induced escalation of drinking. FIG. 2A is a representation of AAV surgeries in mouse NAc. FIG. 2B are fluorescence micrographs if GFP IHC and DNA staining (Hoechst) following AAV-shG9A (G9A knockdown shRNA; 5′-GAGCCACCTCCAGGTGGTTGT-3′; SEQ ID NO: 5). The white scale bar equals 100 microns and the anterior commissure is circled. FIG. 2C is a bar graph of quantitative PCR on viral infused NAc tissue using G9A primers (n=3 for each group). FIG. 2D is an exemplary timeline of experimentation. FIG. 2E is a graph of average drinking during baseline. FIG. 2F is a graph of drinking after three repeated CIE/air exposure cycles. Data are expressed as mean+/−s.e.m. *p<0.05. In FIG. 2D, light gray boxes correspond to 2-bottle choice session timepoints and dark gray boxes correspond to timepoints of No Testing.

FIGS. 3A-3E. NAc G9A knockdown blocks stress-potentiated drinking. FIG. 3A is an exemplary timeline of experiment, which is a continuation of the experiment from FIG. 2. FIG. 3B is a bar graph of drinking 30 minutes after a saline i.p. injection following a 5^{th} CIE/air exposure cycle. FIG. 3C is a bar graph of saline vs. U50,488 comparison for only air-treated controls. FIG. 3D is a bar graph of saline vs. U50,488 comparison for only CIE-treated controls. FIG. 3E is a bar graph of drinking 30 minutes after a 5 mg/kg U50,488 i.p. injection following 3 days of withdrawal. Data are expressed as mean+/−s.e.m. **p<0.01 and ***p<0.001. In FIG. 3A, light gray boxes correspond to 2-bottle choice session timepoints, dark gray boxes that lack any characters above or below correspond to timepoints of No Testing, dark gray boxes with an asterisk above correspond to timepoints of administration of 1.25 mg/kg U50,488, and dark gray boxes with two carets (\hat{ }) below correspond to timepoints of administration of 5 mg/kg U50,488.

FIGS. 4A-4E. NAc G9A knockdown blocks two forms of stress-regulated drinking. FIG. 4A is an exemplary timeline of experiment. FIG. 4B is a graph of average drinking during baseline. FIG. 4C is a bar graph of drinking 30 minutes after a 5 mg/kg U50,488 i.p. injection. FIG. 4D is a bar graph of drinking 30 minutes after predator odor exposure. FIG. 4E is a bar graph of average drinking during a week with no stress testing. Data are expressed as mean+/−s.e.m. *p<0.05, **p<0.01, and ****p<0.0001. In FIG. 4A, light gray boxes correspond to 2-bottle choice session timepoints, dark gray boxes that lack any characters below correspond to timepoints of No Testing, dark gray boxes with a caret below correspond to timepoints of administration of 5 mg/kg U50,488, and dark gray boxes with an asterisk below correspond to timepoints of predator odor exposure.

FIGS. 5A-5D. Systemic administration of a pharmacological G9A inhibitor blocks stress-regulated drinking. FIG. 5A is an exemplary timeline of experiment. FIG. 5B is a graph of average drinking during baseline (weeks 1-2) and following repeated injections (weeks 3-4). FIG. 5C is a bar graph of drinking 30 minutes after a 5 mg/kg U50,488 i.p. injection. FIG. 5D is a bar graph of drinking 30 minutes after an acute injection of UNC0642 and 5 mg/kg U50,488 i.p. injection. Data are expressed as mean+/−s.e.m. *p<0.05, **p<0.01, and ****p<0.0001. In FIG. 5A, light gray boxes correspond to 2-bottle choice session timepoints, dark gray boxes that lack any characters below correspond to timepoints of No Testing, gray boxes with a number sign below correspond to timepoints of administration of 4 mg/kg UNC0642 to the chronic group, the gray box with a caret below corresponds to a timepoint of administration of 4 mg/kg UNC0642 to the acute group, and dark gray boxes with an asterisk above correspond to timepoints of administration of 5 mg/kg U50,488.

FIGS. 6A and 6B. Systemic administration of a pharmacological G9A inhibitor reduces both dependence-induced escalation and stress+dependence-induced escalation of ethanol drinking. FIG. 6A is an exemplary timeline of experiment. FIG. 6B (left panel) is a graph of average drinking in control vs dependent mice following repeated injections of vehicle or a G9A inhibitor. FIG. 6B (right panel) is similar to FIG. 5B except that all mice were exposed to forced-swim stress before access to ethanol drinking. Data are expressed as mean+/−s.e.m. ***p<0.001, and ****p<0.0001. In FIG. 6A, light gray boxes correspond to 2-bottle choice session timepoints and dark gray boxes correspond to timepoints of No Testing. Boxes that have asterisks below correspond to timepoints of vehicle or drug administration.

FIGS. 7A-7E. Systemic EHMT2/G9A Inhibition Suppresses Stress-potentiated Alcohol Drinking and Reduces Anxiety-Like Behavior. FIG. 7A is a graph of average drinking during baseline (weeks 1-3) and following repeated injections (weeks 4-5). FIG. 7B is a bar graph of drinking 30 minutes after a 5 mg/kg U50,488 i.p. injection.

FIG. 7C is a bar graph of the latency to enter the open arms of an elevated plus maze (EPM), measured in seconds. FIG. 7D is a bar graph of the number of seconds spent in the open arms of the EPM. FIG. 7E is a bar graph of the number of entries in the open arms of the EPM. Data are expressed as mean+/−s.e.m. *p<0.05 and **p<0.01.

FIGS. 8A and 8B. Systemic EHMT2/G9A Inhibition Does Not Alter Binge-like Ethanol Drinking. FIG. 8A is a graph of average drinking during baseline (week 1) and following repeated injections (weeks 2-3) during 2-hour alcohol drinking sessions. FIG. 8B is a graph of binge-like drinking during 4-hour alcohol drinking sessions. Data are expressed as mean+/−s.e.m.

FIGS. 9A-9K. Systemic EHMT2/G9A Inhibition Does Not Alter Sucrose Self-Administration Behavior. FIGS. 9A-9E are graphs of the number of sucrose pellets earned, paired nose pokes, unpaired nose-pokes, the discrimination ratio, and timeout responding during sucrose-self-administration following 10 days of repeated injections, respectively. FIGS. 9F-9H are graphs of the number of paired nose pokes, unpaired nose-pokes, and the discrimination ratio during extinction of sucrose-self-administration, respectively. FIGS. 9I-9K are bar graphs of the number of paired nose pokes, unpaired nose-pokes, and the discrimination ratio during the last day extinction of sucrose-self-administration as compared to cue-induced reinstatement of sucrose-seeking, respectively. Data are expressed as mean+/−s.e.m.

### DETAILED DESCRIPTION

The epigenetic enzyme histone methyltransferase G9A (hereinafter “G9A” or in some embodiments “G9a”) is a histone methyltransferase that dimethlyates lysine 9 on histone H3 (referred to as “H3K9me2”). It is exemplified by the humans gene products disclosed in Accession Nos. NM_001289413.1 (SEQ ID NO: 7) and NP_001276342.1 (SEQ ID NO: 8) of the GENBANK® biosequence database. In the adult nucleus accumbens (NAc), G9A regulates multiple behaviors associated with substance use disorder. Described herein is evidence that ethanol dependence in mice, produced by chronic intermittent ethanol (CIE) exposure, reduced both G9A and H3K9me2 levels in the adult NAc, but not in the dorsal striatum. Viral-mediated reduction of G9A in the NAc had no effect on baseline volitional ethanol drinking or escalated ethanol drinking produced by CIE exposure. However, NAc G9A was required for stress-regulated and dependence-induced changes in ethanol drinking, including potentiated ethanol drinking produced by activation of the kappa opioid receptor. Consistent with these findings, it was observed that chronic systemic administration of a G9A inhibitor, UNC0642, also blocked stress-induced escalation of ethanol drinking. In addition, chronic systemic administration of a G9A inhibitor, UNC0642, also blocked dependence-induced escalation of ethanol drinking in the CIE model and also reduced drinking in a combined forced swim stress+dependence model. Together, these findings suggested that chronic ethanol use, similar to other abused substances, produced a NAc-selective reduction in G9A levels, which served to limit stress-induced and dependence-induced changes in alcohol drinking. Moreover, the findings described herein suggested that pharmacological inhibition of G9A might provide a novel therapeutic approach to treat stress-induced alcohol drinking—a major trigger of relapse in individuals suffering from AUD—as well as dependence-induced alcohol drinking.

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. For example, the phrase “a composition” refers to one or more compositions, including a plurality of the same composition. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods. Accordingly, unless indicated to the contrary, the numerical parameters set forth in this specification and attached claims are approximations that can vary depending upon the desired properties sought to be obtained by the presently disclosed subject matter.

As used herein, the term “amino acid” refers to α-amino acids that can be employed in producing the presently disclosed subject matter. There are twenty “standard” amino acids that naturally occur in polypeptides, and these are summarized in Table 1.

TABLE 1
Amino Acid Abbreviations and Codes

| Amino Acid | 3-Letter Code | 1-Letter Code |
|---|---|---|
| Alanine | Ala | A |
| Arginine | Arg | R |
| Asparagine | Asn | N |
| Aspartic Acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and other inactive agents can and likely would be present in the pharmaceutical composition.

With respect to the terms “comprising”, “consisting of”, and “consisting essentially of”, where one of these three terms is used herein, the presently disclosed and claimed subject matter can include the use of either or both of the other two terms. For example, in some embodiments, the presently disclosed subject matter relates to compositions comprising peptides. It would be understood by one of ordinary skill in the art after review of the instant disclosure that the presently disclosed subject matter thus encompasses compositions that consist essentially of the peptides of the presently disclosed subject matter, as well as compositions that consist of the peptides of the presently disclosed subject matter.

The term “subject” as used herein refers to a member of any invertebrate or vertebrate species. Accordingly, the term “subject” is intended to encompass any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals)), and all Orders and Families encompassed therein.

The compositions and methods of the presently disclosed subject matter are particularly useful for warm-blooded vertebrates. Thus, the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

Similarly, all genes, gene names, and gene products disclosed herein are intended to correspond to orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes presented herein, the human amino acid sequences disclosed are intended to encompass homologous genes and gene products from other animals including, but not limited to other mammals, fish, amphibians, reptiles, and birds. Also encompassed are any and all nucleotide sequences that encode the disclosed amino acid sequences, including but not limited to those disclosed in the corresponding GENBANK® biosequence database entries.

### II. Methods for Inhibiting Alcohol and/or Other Substance Consumption

In some embodiments, the presently disclosed subject matter relates to methods for reducing alcohol and/or other substance consumption by a subject. As used herein “substance” or “substances” are psychoactive compounds which can be addictive such as alcohol, caffeine, cannabis, hallucinogens, inhalants, opioids, sedatives, hypnotics, anxiolytics, stimulants, nicotine, and tobacco. In some embodiments, the methods comprise, consist essentially of, or consist of administering to a subject in need thereof a composition comprising, consisting essentially of, or consisting of an effective amount of an inhibitor of a euchromatic histone-lysine N-methyltransferase 2 (EHMT2; also referred to herein as “G9A”) biological activity, whereby alcohol and/or other substance consumption by the subject is reduced as compared to what would have occurred had the subject not been administered the composition.

As used herein, the term “inhibitor” refers to an agent which can decrease the expression and/or activity of a H3K9me2 methyltransferase gene product (e.g., a transcription product and/or a translation product), by in some embodiments at least 10% or more, by in some embodiments 10% or more, in some embodiments 50% or more, in some embodiments 70% or more, in some embodiments 80% or more, in some embodiments 90% or more, in some embodiments 95% or more, or in some embodiments 98% or more. The efficacy of an inhibitor of one or more H3K9me2 methyltransferases, e.g., its ability to decrease the level and/or activity of the target can be determined, e.g., by measuring the level of an expression product of the target and/or the activity of the target. Methods for measuring the level of a given mRNA and/or polypeptide are known to one of skill in the art, e.g., RT-PCR with primers can be used to determine the level of RNA and Western blotting with an antibody (e.g., an anti-EHMT2/G9A antibody; such as but not limited to Cat No. ab185050; Abcam US; Cambridge, Mass., United States of America) can be used to determine the expression level of a polypeptide. The activity of, e.g., a H3K9me2 methyltransferase can be determined using methods known in the art, e.g., using commercially available kits for EHMT2/G9A activity (e.g., Cat No. 52001L; BPS Bioscience, San Diego, Calif., United States of America). In some embodiments, the inhibitor can be an inhibitory nucleic acid; an aptamer; an antibody reagent; an antibody; or a small molecule.

As used herein, the phrase “euchromatic histone-lysine N-methyltransferase 2 (EHMT2)”, also referred to as “G9A”, “KMT1C”, “Histone-Lysine N-Methyltransferase”, “Histone H3-K9 Methyltransferase”, “HLA-B Associated Transcript 8”, “Lysine N-Methyltransferase 1C”, “H3-K9-HMTase 3”, “Chromosome 6 Open Reading Frame 30 (C6orf30)”, “BAT8”, “NG36”, “Histone-Lysine N-Methyltransferase, H3 Lysine-9 Specific 3”, “Ankyrin Repeat-Containing Protein”, “G9A Histone Methyltransferase”, “Em:AF134726.3”, “EC 2.1.1.-”, “NG36/G9a”, and “GAT8”, refers to a genetic locus, a gene, and its products that are exemplified by the human EHMT2 gene, which is located on human chromosome 6 as the complement of nucleotides 31,879,759-31,897,698 of Accession No. NC_000006.12 of the GENBANK® biosequence database (SEQ ID NO: 9). Several transcript variants of human EHMT2/G9A gene products have been identified, which are exemplified by Accession Nos. NM_001289413.1 (SEQ ID NO: 7), NM_006709.5 (SEQ ID NO: 10), NM_025256.7 (SEQ ID NO: 12), NM_001318833.1 (SEQ ID NO: 14), and NM_001363689.1 (SEQ ID NO: 16) of the GENBANK® biosequence database. These Accession Nos. of the GENBANK® biosequence database encode proteins identified as Accession Nos. NP_001276342.1 (SEQ ID NO: 8), NP_006700.3 (SEQ ID NO: 11), NP_079532.5 (SEQ ID NO: 13), NP_001305762.1 (SEQ ID NO: 15), and NP_001350618.1 (SEQ ID NO: 17) of the GENBANK® biosequence database, respectively. The biological activities of the EHMT2/G9A gene include methylation of lysine residues of histone H3. Methylation of H3 at lysine 9 by EHMT2/G9A results in recruitment of additional epigenetic regulators and repression of transcription.

Inhibitors of EHMT2/G9A biological activities include those disclosed in U.S. Patent Application Publication Nos. 2018/0256749, 2020/0054635, and 2020/0113901, each of which is incorporated by reference in its entirety. A particular small molecule EHMT2/G9A inhibitor is 2-(4,4-Difluoropiperidin-1-yl)-6-methoxy-N-[1-(propan-2-yl)piperidin-4-yl]-7-[3-(pyrrolidin-1-yl)propoxy]quinazolin-4-amine, also called UNC0642 (CAS No. 1481677-78-4). UNC0642 is commercially available from Sigma-Aldrich Corp. (Catalog No. SML1037; St. Louis, Mo., United States of America). It has the following structure:

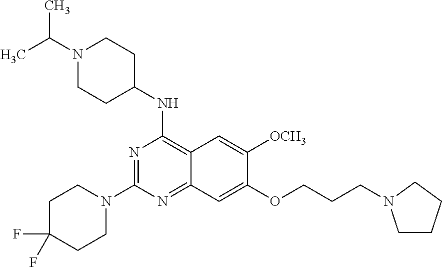 [SMILES: COc1cc2c(NC3CCN(C(C)C)CC3)nc(N3CCC(F)(F)CC3)nc2cc1OCCCN1CCCC1]

Another exemplary small molecule EHMT2/G9A inhibitor is 6-Methoxy-2-morpholin-4-yl-N-(1-propan-2-ylpiperidin-4-yl)-7-(3-pyrrolidin-1-ylpropoxy)quinazolin-4-amine, also referred to as UNC1479. Other exemplary EHMT2/G9A inhibitors include, but are not limited to 2-cyclohexyl-6-methoxy-N-[1-(1-methylethyl)-4-piperidinyl]-7-[3-(1-pyrrolidinyl)propoxy]-4-quinazolinamine; N-(1-isopropylpiperidin-4-yl)-6-methoxy-2-(4-methyl-1,4-diazepan-1-yl)-7-(3-(piperidin-1-yl)propoxy)quinazolin-4-amine; 2-(4,4-difluoropiperidin-1-yl)-N-(1-isopropylpiperidin-4-yl)-6-methoxy-7-(3-(pyrrolidin-1-yl)propoxy)quinazolin-4-amine; or 2-(4-isopropyl-1,4-diazepan-1-yl)-N-(1-isopropylpiperidin-4-yl)-6-methoxy-7-(3-(piperidin-1-yl)propoxy)quinazolin-4-amine, derivatives thereof, metabolic precursors thereof, metabolic products thereof, and/or pharmaceutically acceptable salts thereof. Other EHMT2/G9A inhibitors include 2-(Hexahydro-4-methyl-1H-1,4-diazepin-1-yl)-6,7-dimethoxy-N-(1-(phenylmethyl)-4-piperidinyl)-4-quinazolinamine (also known as Histone Lysine Methyltransferase Inhibitor (CAS 935693-62-2) or BIX 01294 trihydrochloride hydrate), 6-Chloro-N-(4-ethoxyphenyl)-2-methylquinolin-4-amine (also known as CSV0C018875), CPUY074020 (CAS No. 902279-44-1), 2-(benzoylamino)-1-(3-phenylpropyl)-1H-benzimidazole-5-carboxylic acid, methyl ester (also known as BRD4770, CAS No. 1374601-40-7), Chaetocin (CAS No. 28097-03-2), and A-366 (CAS No. 1527503-11-2). Other EHMT2/G9A inhibitors are disclosed in U.S. Patent Application Publication No. 2020/0054635 and U.S. Pat. Nos. 9,284,272 and 9,840,500, each of which is incorporated herein by reference in its entirety.

Also encompassed within the presently disclosed subject matter are derivatives of the disclosed EHMT2/G9A inhibitors. As used herein, the term “derivative” refers to a compound that is structurally similar to but not identical to an EHMT2/G9A inhibitor as disclosed herein and that has at least some EHMT2/G9A inhibitory activity. In some embodiments, an EHMT2/G9A inhibitor is a derivative of UNC0642. See e.g., Liu et al., 2013.

In some embodiments, EHMT2/G9A inhibitors of the presently disclosed subject matter can be metabolic precursors, metabolic products, and/or pharmaceutically acceptable salts of an EHMT2/G9A inhibitors as disclosed herein. As used herein, the term “metabolic precursor” refers to a compound that is metabolized to a biologically active EHMT2/G9A inhibitor of the presently disclosed subject matter in vivo, which in some embodiments can be in vivo in a mammal, including but not limited to a human. As used herein, the term “metabolic product” refers to a compound that results from in vivo metabolism of an EHMT2/G9A inhibitor of the presently disclosed subject matter in order to provide EHMT2/G9A inhibitory activity in a subject. In some embodiments, the metabolic product can be the species that provides the EHMT2/G9A inhibitory activity in vivo, whereas in some embodiments the metabolic product can have some or all of the EHMT2/G9A inhibitory activity in vivo. In some embodiments, some or all of the EHMT2/G9A inhibitor metabolic precursor, the EHMT2/G9A inhibitor, and the EHMT2/G9A inhibitor metabolic product are exposed to metabolic activity in vivo such that the concentrations of each can change within a subject over time.

Inhibition of EHMT2/G9A can also be accomplished using inhibitory nucleic acids. In some embodiments, an inhibitory nucleic acid binds to and partially or completely inhibits processing and/or translation of an RNA gene product of an EHMT2/G9A gene. Exemplary, non-limiting EHMT2/G9A gene products are disclosed herein under various Accession Nos. of the GENBANK® biosequence database, and any subsequence of any of the transcription products of an RNA gene product of an EHMT2/G9A gene can be targeted with an appropriate inhibitory nucleic acid. In some embodiments, the inhibitory nucleic acid is an inhibitory RNA (iRNA). Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the terms inhibitory RNA and “iRNA” refer to an agent that comprises RNA as that term is defined herein, and which mediates the targeted cleavage of an RNA transcript via an RNA-induced silencing complex (RISC) pathway (e.g., an RNA interference (RNAi) pathway). In some embodiments, an iRNA as described herein effects inhibition of the expression and/or activity of a target, e.g., at least one H3K9me2 methyltransferase. In some embodiments, contacting a cell with the inhibitor (e.g., an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA.

In some embodiments, the iRNA can be a double-stranded RNA (dsRNA). A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and in some embodiments fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous subsequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, in some embodiments 15-30 nucleotides in length.

In some embodiments, the RNA component of an iRNA, e.g., a dsRNA, is chemically modified to enhance its stability and/or other beneficial characteristics. The nucleic acids of the presently disclosed subject matter can be synthesized and/or modified by methods well established in the art, such as those described in Current Protocols in Nucleic Acid Chemistry (Beaucage et al., 2002), which is hereby incorporated herein by reference. Modifications include, for example, (a) end modifications (e.g., 5′-end modifications (phosphorylation, conjugation, inverted linkages, etc.) and/or 3′-end modifications (e.g., conjugation, DNA nucleotides, inverted linkages, etc.), (b) base modifications (e.g., replacement of one or more cases with stabilizing bases, destabilizing bases, and/or bases that base pair with an expanded repertoire of partners; (c) removal of bases (abasic nucleotides); conjugated bases; sugar modifications (e.g., at the 2′ position or 4′ position) or replacement of the sugar; as well as (d) backbone modifications, including modification and/or replacement of the phosphodiester linkages. Specific examples of RNA compounds useful in the embodiments described herein include, but are not limited to, RNAs containing modified backbones or no natural internucleoside linkages. RNAs having modified backbones include, among others, those that do not have a phosphorus atom in the backbone. For the purposes of this disclosure, and as sometimes referenced in the art, modified RNAs that do not have a phosphorus atom in their internucleoside backbone can also be considered to be oligonucleosides. In some embodiments, a modified RNA comprises a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Reissue Pat. Ser. No. RE39464, each of which is herein incorporated by reference in its entirety.

Modified RNA backbones that do not include a phosphorus atom therein have backbones that are formed by short chain alkyl or cycloalkyl internucleoside linkages, mixed heteroatoms, and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, each of which is herein incorporated by reference in its entirety.

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference in its entirety. Further teaching of PNA compounds can be found, for example, in Nielsen et al., 1991.

Some embodiments featured in the invention include RNAs with phosphorothioate backbones and oligonucleosides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂—CH₂-[wherein the native phosphodiester backbone is represented as —O—P—O—CH₂-] of the above referenced U.S. Pat. No. 5,489,677, and the amide backbones of the above referenced U.S. Pat. No. 5,602,240. In some embodiments, the RNAs featured herein have morpholino backbone structures of the above referenced U.S. Pat. No. 5,034,506.

Modified RNAs can also contain one or more substituted sugar moieties. The iRNAs, e.g., dsRNAs, featured herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH₂)ₙO]mCH₃, O(CH₂)ₙOCH₃, O(CH₂)ₙNH₂, O(CH₂)ₙCH₃, O(CH₂)ₙONH₂, and O(CH₂)ₙON[(CH₂)ₘCH₃)]₂, where n and m are from 1 to about 10. In other embodiments, dsRNAs include one of the following at the 2′ position: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′ methoxyethoxy (2′-O—CH₂CH₂OCH₃, also known as 2′-O-(2-methoxyethyl) or 2′-MOE) (Martin et al., 1995) i.e., an alkoxy-alkoxy group. Another exemplary modification is 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in U.S. Patent Application Publication No. 2019/0136199, which is incorporated herein by reference in its entirety.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bronco, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Herdewijn, 2008); those disclosed in Kroschwitz, 1990; these disclosed by Englisch et al., 1991; and those disclosed by Sanghvi, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine, 5-methylcytosine substitutions have been shown to increase nucleic acid duplex stability by 0.6-1.2° C. (Sanghvi, 1993) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference in its entirety.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen et al., 2005; Mook et al., 2007; Grunweller et al., 2003). Representative U.S. patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the RNA of an iRNA featured in the invention involves chemically linking to the RNA one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., 1989), cholic acid (Manoharan et al., 1994), a thioether, e.g., beryl-S-tritylthiol (Manoharan et al., 1992; Manoharan et al., 1993), a thiocholesterol (Oberhauser et al., 1992), an aliphatic chain, e.g., dodecandiol or undecyl residues (Saison-Behmoaras et al., 1991; Kabanov et al., 1990; Svinarchuk et al., 1993), a phospholipid, e.g., di-hexadecyl-rac-glycerol or triethyl-ammonium 1,2-di-O-hexadecyl-rac-glycero-3-phosphonate (Manoharan et al., 1995a; Shea et al., 1990), a polyamine or a polyethylene glycol chain (Manoharan et al., 1995b), or adamantane acetic acid (Manoharan et al., 1995a), a palmityl moiety (Mishra et al., 1995), and/or an octadecylamine or hexylamino-carbonyloxycholesterol moiety (Crooke et al., 1996).

In some embodiments, a nucleic acid as described herein is comprised by a vector. In some of the aspects described herein, a nucleic acid sequence as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments, an adeno-associated virus (AAV2) viral-mediated short hairpin RNA (shRNA) interference approach (e.g., an AAV vector encoding a shRNA targeting a G9a gene product; including but not limited to AAV-shG9a; an AAV vector comprising SEQ ID NO: 5) as described in Anderson et al., 2019 and in the EXAMPLES below can be employed. Other inhibitory nucleic acids targeting EHMT2/G9A gene products can also be designed and employed as EHMT2/G9A inhibitors. By way of example and not limitation, the human EHMT2/G9A genetic locus is found on human chromosome 6 and corresponds to the complement of nucleotides 31,879,759-31,897,698 of Accession No. NC_000006.12 of the GENBANK® biosequence database (SEQ ID NO: 9). This locus encodes several alternative polypeptides, including but not limited to Accession Nos. XP_006715037.1 (SEQ ID NO: 18), XP_006715038.1 (SEQ ID NO: 19), NP_001382089.1 (SEQ ID NO: 20), NP_001382092.1 (SEQ ID NO: 21), NP_001276342.1 (SEQ ID NO: 22), NP_001305762.1 (SEQ ID NO: 15), NP_001350618.1 (SEQ ID NO: 17), NP_006700.3 (SEQ ID NO: 11), and NP_079532.5 (SEQ ID NO: 13) of the GENBANK® biosequence database. The GENBANK® biosequence database also includes five reference nucleotide sequences for transcription products of the EHMT2/G9A genetic locus, which are Accession Nos. NM_001289413.1 (SEQ ID NO: 7), NM_001318833.1 (SEQ ID NO: 14), NM_001363689.1 (SEQ ID NO: 16), NM_006709.5 (SEQ ID NO: 10), and NM_025256.7 (SEQ ID NO: 12). Based on the nucleotide sequences of these transcription produces, one of ordinary skill in the art can design numerous inhibitory nucleic acids that target human EHMT2/G9A gene products. Similar approaches can be taken for targeting EHMT2/G9A gene products from other species based on sequences found in the GENBANK® biosequence database, including such species as mouse (exemplary transcripts can be found at Accession Nos. NM_145830.3 (SEQ ID NO: 27, encoding Accession No. NP_665829.1; SEQ ID NO: 28) and NM_001286573.2 (SEQ ID NO: 25, encoding Accession No. NP_001273502.1; SEQ ID NO: 26) of the GENBANK® biosequence database), rat (exemplary transcript can be found at Accession No. NM_212463.1 (SEQ ID NO: 29, encoding Accession No. NP_997628.1; SEQ ID NO: 30) of the GENBANK® biosequence database), Equus caballus (exemplary transcript can be found at Accession No. XM_023624646.1 (SEQ ID NO: 31, encoding Accession No. XP_023480414.1; SEQ ID NO: 32) of the GENBANK® biosequence database), Bos taurus (exemplary transcript can be found at Accession No. NM_001206263.2 (SEQ ID NO: 23, encoding Accession No. NP_001193192.2; SEQ ID NO: 24) of the GENBANK® biosequence database), etc.

In some embodiments, EHMT2/G9A inhibitors of the presently disclosed subject matter can be an antibody that binds to an EHMT2/G9A polypeptide and/or a fragment or derivative thereof that comprises an antigen-binding domain (i.e., a paratope) that binds to an EHMT2/G9A polypeptide. In some embodiments, one or more antibodies or fragments thereof are used. In some embodiments, one or both antibodies are single chain, monoclonal, bi-specific, synthetic, polyclonal, chimeric, human, or humanized, or active fragments or homologs thereof. In some embodiments, the antibody binding fragment is scFV, F(ab′)₂, F(ab)₂, Fab′, or Fab. Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′)₂, and single chain Fv (scFv) fragments. In some embodiments, the specific binding molecule is a single-chain variable (scFv). The specific binding molecule or scFv may be linked to other specific binding molecules (for example other scFvs, Fab antibody fragments, chimeric IgG antibodies (e.g., with human frameworks)) or linked to other scFvs of the presently disclosed subject matter so as to form a multimer which is a multi-specific binding protein, for example a dimer, a trimer, or a tetramer. Bi-specific scFvs are sometimes referred to as diabodies. Fragments within the scope of the term “antibody” include those produced by digestion with various proteases, those produced by chemical cleavage and/or chemical dissociation and those produced recombinantly, so long as the fragment remains capable of specific binding to a target molecule (i.e., comprise at least one paratope). Representative patent documents disclosing techniques relating to antibody production include the following, all of which are herein incorporated by reference in their entireties: PCT International Patent Application Publication Nos. WO 1992/02190 and WO 1993/16185; U.S. Patent Application Publication Nos. 2004/0253645, 2003/0153043, 2006/0073137, 2002/0034765, and 2003/0022244; and U.S. Pat. Nos. 4,816,567; 4,946,778; 4,975,369; 5,001,065; 5,075,431; 5,081,235; 5,169,939; 5,202,238; 5,204,244; 5,225,539; 5,231,026; 5,292,867; 5,354,847; 5,436,157; 5,472,693; 5,482,856; 5,491,088; 5,500,362; 5,502,167; 5,530,101; 5,571,894; 5,585,089; 5,587,458; 5,641,870; 5,643,759; 5,693,761; 5,693,762; 5,712,120; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,548,640; 6,632,927; 6,639,055; 6,750,325; and 6,797,492. Commercially available anti-EHMT2/G9A antibodies include those sold by Abcam US (Cambridge, Mass., United States of America; e.g., Catalog Nos. ab 185050, ab 133482, ab 240289, ab 229455, ab 183889, ab 40542, ab 248517, and ab 218359), Proteintech North America (Rosemont, Ill., United States of America; Catalog No. 66689-1-1g); Thermo Fisher Scientific (Waltham, Mass., United States of America; e.g., Catalog Nos. MA5-14880, PA5-34971, PA5-78347, MA5-38514, MA5-36145, PA5-111317, PA5-40552, and others), and Novus Biologicals LLC (Centennial, Colo., United States of America; Catalog No. NB100-40825).

As disclosed herein, modulation of EHMT2 biological activities can result in a reduction in stress-related and/or dependence-related alcohol consumption. As such and since stress is associated with relapse in subjects with Alcohol Use Disorder (AUD), in some embodiments the presently disclosed subject matter also relates to methods for reducing relapse vulnerability in AUD subjects. In some embodiments, the methods comprise, consist essentially of, or consist of administering to a subject suffering from AUD a composition comprising, consisting essentially of, or consisting of an effective amount of an inhibitor of a euchromatic histone-lysine N-methyltransferase 2 (EHMT2/G9A) biological activity, whereby the effective amount is sufficient to reduce the incidence of stress-related alcohol consumption by the subject as compared to what would have occurred had the subject not been administered the composition.

In any of the methods of the presently disclosed subject matter, in some embodiments administration of the EHMT2/G9A inhibitors results in a reduction of dimethylation of lysine 9 of histone H3 (H3K9me2) in the subject, optionally a reduction of dimethylation of lysine 9 of histone H3 (H3K9me2) in the nucleus accumbens (NAc) in the subject.

In some embodiments, the presently disclosed subject matter provides the use of EHMT2/GA9 inhibition in relapse-like behavior for substance use disorders. In some embodiments, the UNC0642 compound is used to treat one or more subjects having on one more such behaviors. However, any composition as disclosed herein can be employed in such treatment methods and uses.

In some embodiments, the methods and compositions of the presently disclosed subject matter are used for treating individuals with co-morbid psychiatric diseases and/or disorders and SUD and/or AUD, e.g., a SUD, optionally AUD. Thus, in some embodiments, the presently disclosed subject matter treats subjects having an SUD and/or AUD and a co-morbid psychiatric disorder. In some embodiments, “co-morbid psychiatric diseases and/or disorders” as used herein refers to stress- and/or anxiety-related disorders and/or disorders exacerbated by stress and anxiety. In accordance with some embodiments of the presently disclosed subject matter, G9a inhibition can be useful in treating symptoms in individuals diagnosed with stress- and/or anxiety-related disorders and/or disorders exacerbated by stress and anxiety, including post-traumatic stress disorder (PTSD), panic disorder, social anxiety disorder, general anxiety disorder, and major depressive disorder, in some embodiments where these disorders are co-morbid with SUD and/or AUD in a subject, e.g., a substance use disorder (SUD), optionally Alcohol Use Disorder (AUD).

The compositions of the presently disclosed subject matter comprise in some embodiments a composition that includes an EHMT2/G9A inhibitor as disclosed herein and a carrier, particularly a pharmaceutically acceptable carrier, such as but not limited to a carrier pharmaceutically acceptable for use in humans. Any suitable pharmaceutical formulation can be used to prepare the compositions for administration to a subject.

For example, suitable formulations can include aqueous and non-aqueous sterile injection solutions that can contain antioxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

Suitable methods for administration of the compositions of the presently disclosed subject matter include, but are not limited to intravenous administration and delivery directly to a target tissue or organ. Exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated. By way of example and not limitation, in some embodiments a composition of the presently disclosed subject matter is administered to the subject via a route selected from the group consisting of intraperitoneal, intramuscular, intravenous, and intranasal, or any combination thereof.

In addition, with respect to subcutaneous delivery, a pen delivery device readily has applications in delivering a pharmaceutical composition of the presently disclosed subject matter. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with anew cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, United Kingdom), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind., United States of America), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J., United States of America), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif., United States of America), the PENLET™ (Haselmeier, Stuttgart, Germany), the EPI PEN (Dey, L.P.), and the HUMIRA™ Pen (Abbott Labs, Abbott Park, Ill., United States of America), to name only a few. See e.g., U.S. Pat. Nos. 7,762,994; 8,409,149; 8,556,864; 8,579,869; 9,011,391; and 9,265,893, the disclosure of each of which is incorporated herein by reference in its entirety.

The methods described herein use pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethylene glycol, hyaluronic acid, ethanol, cyclodextrins, modified cyclodextrins (i.e., sulfobutyl ether cyclodextrins), etc. Suitable excipients for non-liquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, and salts is available in Remington's Pharmaceutical Sciences, 1990.

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions, or the like, preferably in unit dosage form suitable for single administration of a precise dosage. The compositions will include an effective amount of the selected drug in combination with a pharmaceutically acceptable carrier and, in addition, may include other pharmaceutical agents, adjuvants, diluents, buffers, etc.

In some embodiments, the mode of administration is a solid dosage form, such as tablets and pills that are orally administered.

The EHMT2/G9A inhibitor-based therapies of the presently disclosed subject matter can thus be provided by several routes of administration. In some embodiments, intracardiac muscle injection is used, which avoids the need for an open surgical procedure. The EHMT2/G9A inhibitors can in some embodiments be introduced in an injectable liquid suspension preparation or in a biocompatible medium that is injectable in liquid form and becomes semi-solid at the site of administration. The injectable liquid suspension EHMT2/G9A inhibitor preparations can also be administered intravenously, either by continuous drip or as a bolus.

As such, suitable methods for administration of the compositions of the presently disclosed subject matter include, but are not limited to intravenous administration and delivery directly to a target tissue or organ. In some embodiments, the method of administration encompasses features for regionalized delivery or accumulation of the compositions of the presently disclosed subject matter at the site in need of treatment. In some embodiments, the compositions of the presently disclosed subject matter are delivered directly into the tissue or organ to be treated, such as but not limited to the nervous system.

Injection medium can be any pharmaceutically acceptable isotonic liquid. Examples include phosphate buffered saline (PBS), culture media such as X-vivo medium, DMEM (in some embodiments serum-free), physiological saline, 5% dextrose in water (D5W), or any biocompatible injectable medium or matrix.

A pharmaceutical composition as described herein can be administered once, twice, three times, or more. In some embodiments, the pharmaceutical composition is administered to the subject on at least two separate occasions. In some embodiments, pharmaceutical composition is administered to the subject chronically, which in some embodiments includes one or more doses a day for 1, 2, 3, 4, 5, 6, 7, 8, 9, or 10 days or more. In those embodiments wherein the pharmaceutical composition is administered to the subject in two or more doses covering multiple occasions, the time between the administrations of the doses can be hours, days, weeks, or months.

An effective dose of a composition of the presently disclosed subject matter is administered to a subject in need thereof. A “treatment effective amount”, “therapeutic amount”, or “effective amount” as those phrases are used herein is an amount of a therapeutic composition sufficient to produce a measurable response (e.g., a biologically or clinically relevant response in a subject being treated). Actual dosage levels of an active agent or agents (e.g., EHMT2/G9A inhibitors) in the compositions of the presently disclosed subject matter can be varied so as to administer an amount of the active agent(s) that is effective to achieve the desired therapeutic response for a particular subject. Exemplary, non-limiting dosages include about 1.0 mg/kg administered i.p., about 1.25 mg/kg administered i.p., about 1.5 mg/kg administered i.p., about 1.75 mg/kg administered i.p., about 2.0 mg/kg administered i.p., about 2.25 mg/kg administered i.p., about 2.5 mg/kg administered i.p., about 2.75 mg/kg administered i.p., about 3.0 mg/kg administered i.p., about 3.25 mg/kg administered i.p., about 3.5 mg/kg administered i.p., about 3.75 mg/kg administered i.p., about 4.0 mg/kg administered i.p., or greater than 4.0 mg/kg administered i.p. It is noted that a dosage of 2.0 mg/kg administered i.p. was found to be effective in the mouse models disclosed herein for both male and female subjects, and as such, in some embodiments a dosage of about 2.0-4.0 mg/kg administered i.p. would be an appropriate dose.

The selected dosage level can depend upon the activity of the therapeutic composition, the route of administration, combination with other drugs or treatments, the severity of the condition being treated, the condition and prior medical history of the subject being treated, and the genus and species of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore a “treatment effective amount” can vary. However, one skilled in the art can readily assess the potency and efficacy of a therapeutic composition of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular injury treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods for the EXAMPLES

Animal Care. Adult male C57BL/6J mice (Jackson Laboratory, Bar Harbor, Me.) were singly housed in a climate-controlled environment (21° C.) on a 12h light-dark cycle. Animals were habituated to the housing environment for at least 7 days prior to use in experiments, and had food and water ad libitum. All drinking and behavioral experiments were performed during the dark cycle as described below, and were approved by the MUSC Institutional Animal Care and Use Committee (IACUC) in facilities accredited by the American Association for the Accreditation of Laboratory Animal Care (AAALAC). All procedures were conducted in accordance with the guidelines established by the National Institutes of Health and the National Research Council.

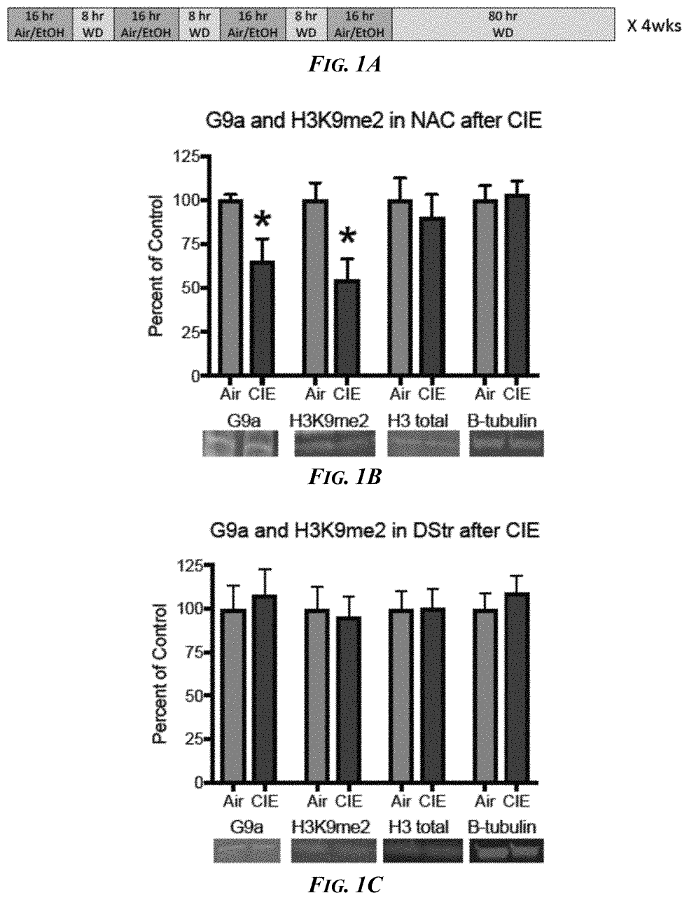

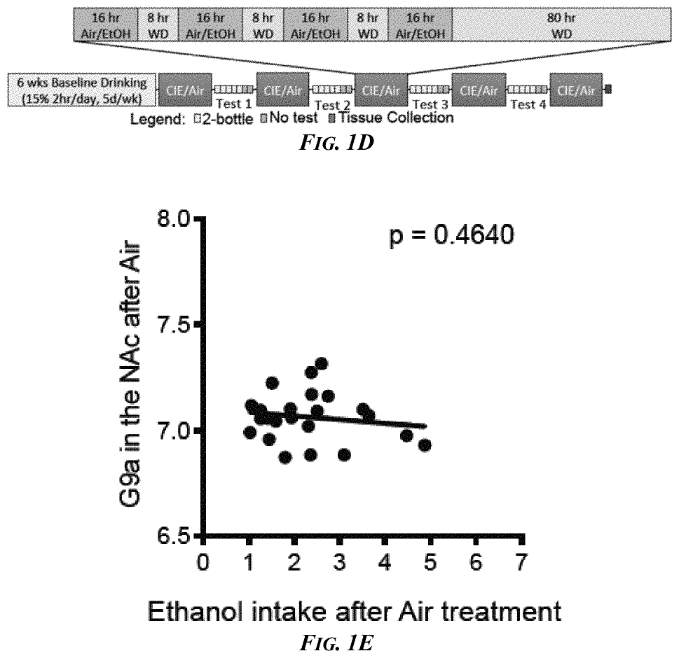

EHMT2/G9A Microarray Analysis. A previously published microarray dataset (see the website of GeneNetwork; www.genenetwork.org) was analyzed for EHMT2/G9A mRNA changes in chronic intermittent ethanol (CIE) exposure vs. air-exposed BXD mice interspersed with limited access 2-bottle choice drinking as described previously (Lopez et al., 2017; Rinker et al., 2017; van der Vaart et al., 2017) and as illustrated in FIG. 1A. EHMT2/G9A mRNA levels were correlated with ethanol intake after the final week of baseline drinking, and after four test periods following CIE or Air exposure.

CIE induction for western blotting. C57BL/6J male mice were exposed ethanol vapors in inhalation chambers to induce dependence, or to air in control chambers, for 5 cycles (16 hours/day×4 days/week) as illustrated in Table 2 according to established methodology (Badanich et al., 2011; den Hartog et al., 2016). 72-96 hours following the last exposure, these mice were euthanized and the NAc (ventral striatum: core and shell) and dorsal striatum were harvested. Tissues were pooled from 3 mice and frozen on dry ice.

TABLE 2
R-squared and p-values for Correlations Between EHMT2/G9A
mRNA Levels in the NAc Following Air Exposure

| EHMT2/G9A Levels in the NAc | R2 | p-value |
|---|---|---|
| Air-treated - baseline | 0.0155 | 0.6534 |
| Air-treated - Test 1 | 0.1147 | 0.1055 |
| Air-treated - Test 2 | 0.0930 | 0.1571 |
| Air-treated - Test 3 | 0.0246 | 0.4640 |
| Air-treated - Test 4 | 0.1201 | 0.0971 |

Western blotting. Tissue was lysed, immunoblotted according to previously published methods (Taniguchi et al., 2017), and analyzed by western blot for EHMT2/G9A, H3K9, H3K9me2, and Tubulin Beta 3 as a loading control. Primary antibodies were anti-EHMT2/G9A (Research Resource Identifier (RRID): AB_731483, Catalog No. ab40542, Abcam US, Cambridge, Mass., United States of America, rabbit, 1:4000), anti-Histone H3 (RRID: AB_331563, Catalog No. 9715S, Cell Signaling Technology, Danvers, Mass., United States of America, rabbit, 1:10,000), anti-H3K9me2 (RRID: AB_449854, Catalog No. ab1220, Abcam US, mouse, 1:10,000), and anti-Tubulin Beta 3 (RRID: AB_10063408, Catalog No. 801202, BioLegend, San Diego, Calif., United States of America, mouse, 1:50,000). Secondary antibodies: 680RD anti-rabbit (RRID: AB_10956166, Catalog No. 926-68071, LI-COR Biosciences, Lincoln, Nebr., United States of America, goat, 1:10,000) and 800CW anti-mouse (RRID: AB_621842, Catalog No. 926-32210, LI-COR, goat, 1:10,000). Blots were developed on a LI-COR Odyssey CLx and analyzed with ImageStudio software.

Viral vectors. EHMT2/G9A was knocked down by using a previously validated adeno-associated vector serotype 2 vector encoding a short hairpin RNA (AAV-shG9A; comprising 5′-GAGCCACCTCCAGGTGGTTGT-3′; SEQ ID NO: 5). The control virus was a scrambled version of this sequence with no known homology (AAV-shSC; encoding 5′-AAATGTACTGCGCGTGGAGAC-3′; SEQ ID NO: 6). These viruses have been previously characterized via western blotting (Anderson et al., 2019) and are further characterized below.

Stereotaxic surgery. C57BL/6J male mice underwent isoflourane-anesthetized survival surgery to microinject AAV-shG9A (encoding SEQ ID NO: 5) or AAV-shSC (encoding SEQ ID NO: 6) bilaterally into the NAc (AP: +1.6, DV: −4.4 ML: +1.5, 10° angle) and allowed at least 7 days of recovery. Carprofen (5 mg/kg, once daily for 3 days) was used for post-surgical pain relief.

Immunohistochemistry (IHC). Brains from virus-infused mice were drop fixed in 4% paraformaldehyde at least three weeks following surgery to allow for peak AAV expression. Following at least a 24 hour post-fix, brains were cryoprotected with 30% sucrose and sliced at 60 microns on a microtome. Tissue was blocked in buffer (3% bovine serum albumen, 1.5% normal donkey serum, 0.2% Triton-X, 0.2% Tween-20 in PBS) for at least 1 hour, and then transferred to new buffer with anti-GFP (RRID:AB_10000240, Catalog No. GFP-1020, Aves Labs, Davis, Calif., United States of America, chicken, 1:4000). The next day, tissue was washed 3×5 minutes, and anti-chicken secondary was added (RRID:AB_2340375, Catalog No. 703-545-155, 488 donkey anti-chicken, Jackson ImmunoResearch Inc., West Grove, Pa., United States of America, 1:500). Tissue was washed in bisbenzimide (1:5000, Hoechst 33342, Invitrogen Corp. Carlsbad, Calif., United States of America) for 2 minutes, followed by 2×5 mins PBS washes, and then mounted. Images were taken with a Nikon Eclipse 80i fluorescent microscope and processed with ImageJ (RRID:SCR_002285, Fiji, NIH; Schneider et al., 2012).

Quantitative Polymerase Chain Reaction (qPCR). Virus-infused mice were euthanized following stereotaxic surgery and fresh NAc tissue was harvested at least three weeks following surgery. Native GFP signal was used to localize tissue punches. mRNA was extracted using QIAzol Lysis Reagent (Catalog No. 56008534, QIAGEN LLC-USA, Germantown, Md., United States of America) and the RNeasy Micro Kit (Catalog No. 74004, QIAGEN). qPCR was performed using a Biorad CFX96 using G9A primers (Forward: TGCCTATGTGGTCAGCTCAG (SEQ ID NO: 1); Reverse: GGTTCTTGCAGCTTCTCCAG (SEQ ID NO: 2) and normalized to GAPDH (Forward: AGGTCGGTGTGAACGGATTTG (SEQ ID NO: 3); Reverse: TGTAGACCATGTAGTTGAGGTCA (SEQ ID NO: 4).

Two-bottle choice ethanol drinking. Following stereotaxic surgery, mice received two-bottle choice (15% (v/v) ethanol vs. water) testing starting 3 hours after lights off for 4 weeks (2 bhr/d, 5 bd/wk), and then 5 cycles of CIE or air exposure interspersed with weekly test drinking sessions starting 72 hours after CIE (or Air) exposure (Becker & Lopez, 2004; Lopez & Becker, 2005; Griffin et al., 2009; Lopez et al., 2017).

Kappa agonist injections prior to drinking. As noted in FIG. 3A, two-bottle choice testing was interrupted between the fourth and fifth CIE cycle due to Hurricane Florence. Following the fifth CIE cycle, mice received 2 days of saline i.p. injections 1 hour before drinking, followed by 2 days of 1.25 mg/kg (trans-(1R,2R)-3,4-Dichloro-N-methyl-N-[2-(1-pyrrolidinyl)cyclohexyl]-benzeneacetamide (U50,488; CAS No. 67197-96-0) as previously described (Anderson et al., 2016a). On the fifth day, mice received a 5 mg/kg dose of U50,488. Finally, following a 3 day withdrawal period, mice were injected with a dose of 5 mg/kg U50,488 before drinking.

Placement verification. Mice were rapidly sacrificed and brains were drop-fixed in 4% paraformaldehyde for at least 24 hours before transferring to 30% sucrose for at least 3 days, slicing on a microtome, and mounting on slides. Native GFP fluorescence was used to verify proper placement under blinded conditions. Only mice with bilateral NAc GFP expression were included in the final analysis.

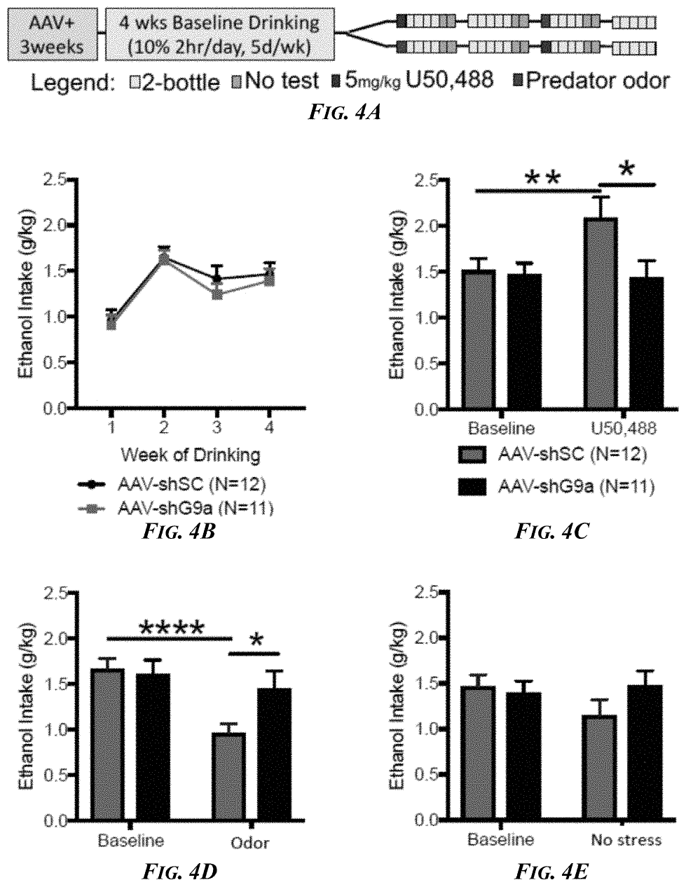

U50,488 and predator odor stress. As illustrated in FIG. 4A, mice underwent stereotaxic surgery and two-bottle choice testing for 4 weeks (10% (v/v) ethanol vs. water, 2 h/d, 5 d/wk). In this experiment bottles were presented 30 minutes before lights off Mice were then split into even groups for stress-testing in a counterbalanced design. The first group had a single 5 mg/kg U50,488 i.p. injection following 3 days of withdrawal and two-bottle choice testing was performed 30 minutes later and then daily for the next 2 weeks. The following Monday, mice were moved to a separate room and were placed in anew cage filled with dirty rat bedding for 30 minutes following previously described methods (Cozzoli et al., 2014), then immediately returned to their normal housing room for drinking. Two-bottle choice was assessed for 2 weeks and then mice were euthanized and viral placements were examined. The other group had predator odor exposure first followed by U50,488 injections before euthanizing.

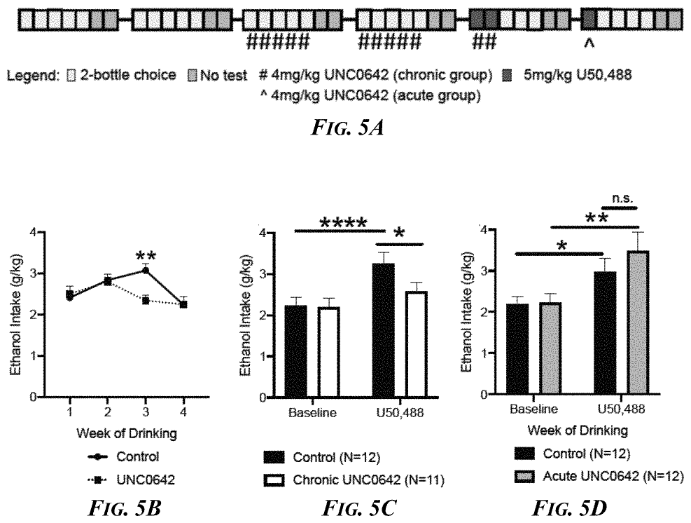

Systemic G9A inhibitor administration and stress-potentiated drinking. As shown in FIG. 5A, mice were given 2 weeks of baseline drinking using two-bottle choice (10% (v/v) ethanol vs. water, 2 h/d, 5 d/wk) with saline injections 30 minutes before each session. Mice were then divided into 2 even groups and given chronic injections of either vehicle or 4 mg/kg UNC0642 for 2 weeks similar to a previous in vivo study in mice (Wang et al., 2018). Next, following 3 days of withdrawal, control mice were injected with 5 mg/kg U50,488 only and chronic treated mice were given both U50,488 and UNC0642 in the same injection 30 minutes before the session. This test was repeated again the following day and then only vehicle injections were given to all mice for the next 3 days before two-bottle choice testing. Finally, after another 3 day withdrawal, an acute injection of UNC0642+U50,488 was given to the naïve control group while U50,488 alone was administered to the chronic UNC0642 group 30 minutes before drinking.

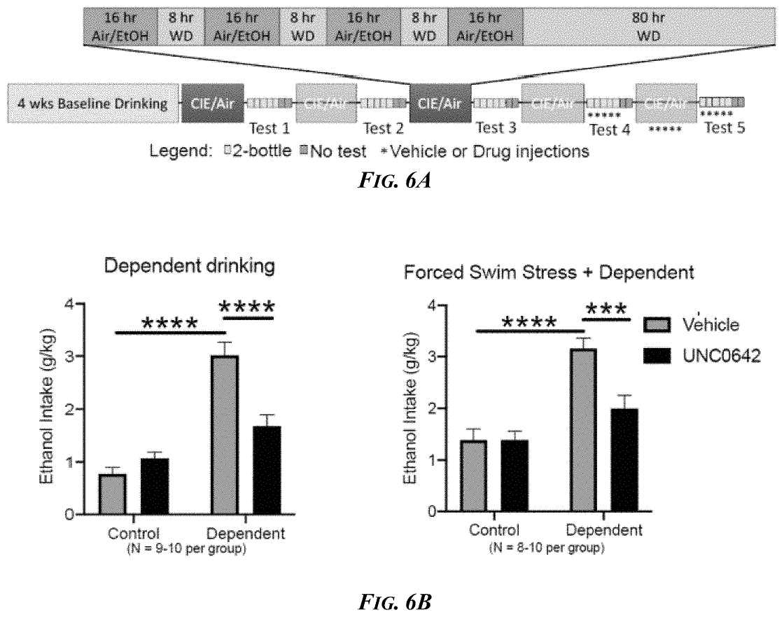

Systemic G9A inhibitor administration and dependence-potentiated and stress+dependence-potentiated drinking. As shown in FIG. 6A, mice were given 4 weeks of baseline drinking using two-bottle choice (10% (v/v) ethanol vs. water, 2h/d, 5d/wk). Mice were then divided into 4 even groups. Mice were exposed to Air/No stress, Air/FSS, CIE/No Stress, or CIE/stress in a 2×2 design to assess the effects of UNC0642 on dependence drinking alone and dependence+stress. Chronic injections of either vehicle or 4 mg/kg UNC0642 were started during the “test 4” phase in FIG. 6A and continued for 15 days. Ethanol consumed was recorded daily.

Statistics. Microarray data were analyzed with linear regression. T-tests were used to analyze protein and mRNA differences. One-way, two-way, or three-way ANOVAs were used to analyze behavioral data where appropriate. For three-way ANOVAs, only significant main effects and interactions are reported below. Fishers LSD post-hoc tests were used following significant ANOVAs. A Grubbs outlier test was applied where appropriate. All statistics were performed with GraphPad Prism 8 and p<0.05 was considered significant.

### Example 1

### EHMT2/G9A Levels in NAc are Negatively Regulated by Chronic Ethanol Exposure

To test whether chronic ethanol exposure regulates EHMT2/G9A levels in the NAc, we isolated brain tissues (NAc or dorsal striatum) after 4 weeks of Air vs. CIE exposure (FIG. 1A) and analyzed EHMT2/G9A protein levels by immunoblotting. We observed that CIE treatment produced a significant reduction (>30%) in NAc EHMT2/G9A protein levels (FIG. 1B; t-test: t(13)=2.471, p=0.028).

We next examined the consequences of the 4-week CIE treatment on H3K9me2, a major substrate affected by EHMT2/G9A's enzymatic activity. Similar to the CIE-induced reduction of EHMT2/G9A protein levels in NAc, we detected a significant reduction (˜40%) of H3K9me2 levels (FIG. 1B, t(13)=2.916, p=0.0120). However, no changes in total histone H3 protein (t(13)=0.5383, p=0.5995) or Beta-Tubulin (t(13)=0.3121, p=0.7599) were observed (FIG. 1B). These reductions in EHMT2/G9A and H3K9me2 did not occur broadly since no changes were observed in dorsal striatum of the same animals (FIG. 1C; EHMT2/G9A (t(13)=0.4089, p=0.6892), H3K9me2 (t(13)=0.2775, p=0.7858), histone H3 (t(13)=0.9818, p=0.9818), β-tubulin (t(13)=0.7212, p=0.4836). These results indicate that chronic ethanol exposure reduces NAc EHMT2/G9A and histone H3K9me2, and that these changes are brain region selective.

Since CIE exposure produced changes in NAc EHMT2/G9A levels, we next asked whether EHMT2/G9A mRNA levels in Air or CIE-treated animals correlated with levels of ethanol drinking. Using a published dataset of NAc gene expression in Air vs. CIE-treated mice (Lopez et al., 2017; Rinker et al., 2017; van der Vaart et al., 2017)), we observed that Air-only control animals showed no significant correlation between ethanol drinking and NAc EHMT2/G9A mRNA levels at any time point assessed (FIGS. 1D and 1E and Table 2). However, CIE treatment produced a significant negative correlation between EHMT2/G9A mRNA levels in NAc and ethanol intake at every post-CIE drinking timepoint (FIGS. 1D and 1F and Table 3), suggesting that CIE might negatively regulate EHMT2/G9A levels in the NAc and influence ethanol drinking.

TABLE 3
R-squared and p-values for Correlations Between EHMT2/G9A
mRNA Levels in the NAc Following CIE Exposure

| EHMT2/G9A Levels in the NAc | R2 | p-value |
|---|---|---|
| CIE-treated - baseline | 0.0017 | 0.8452 |
| CIE-treated - Test 1 | 0.1733 | 0.0308 |
| CIE-treated - Test 2 | 0.2051 | 0.0177 |
| CIE-treated - Test 3 | 0.2463 | 0.0085 |
| CIE-treated - Test 4 | 0.1785 | 0.0281 |

### Example 2

### Reduction of NAc EHMT2/G9A Levels is Not Sufficient to Modulate Ethanol Drinking

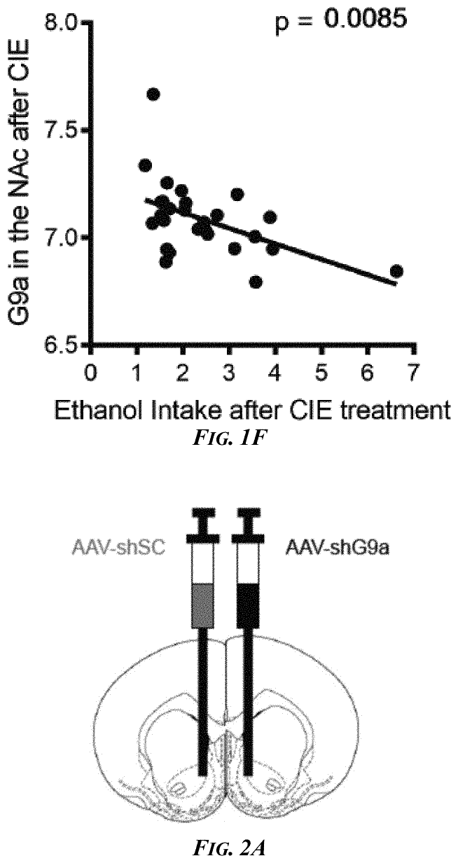

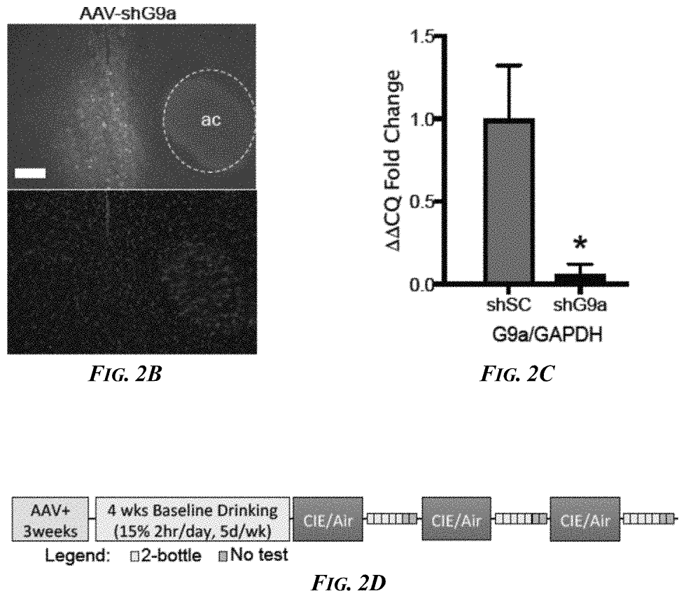

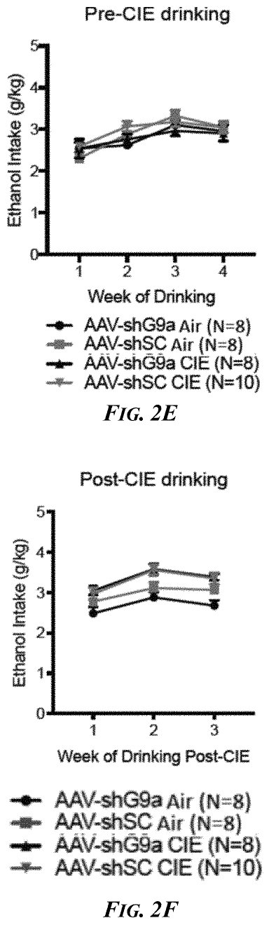

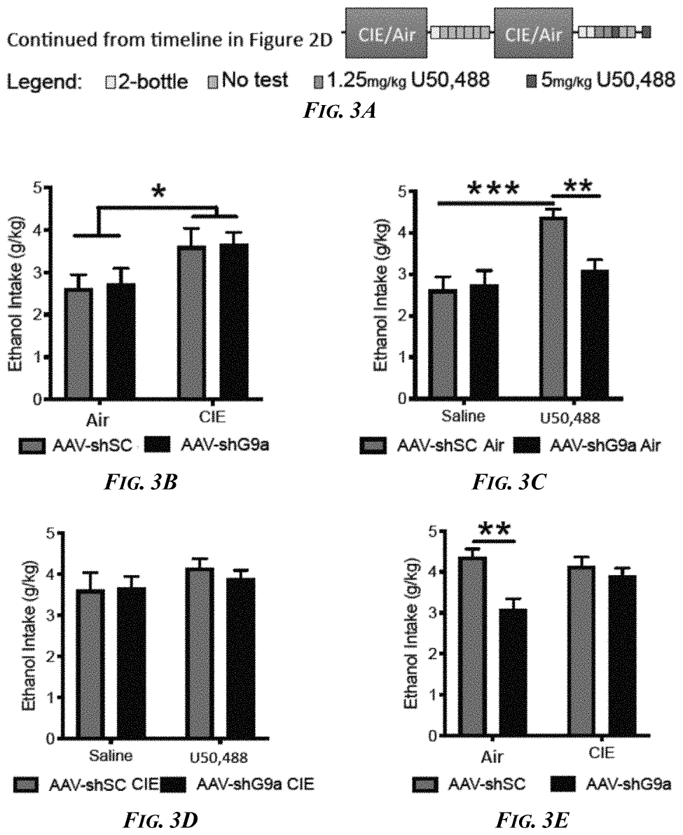

To determine if CIE-induced reduction of EHMT2/G9A levels in the adult NAc promote escalated ethanol drinking, we utilized an adeno-associated virus (AAV2) viral-mediated short hairpin RNA interference approach (AAV-shG9A; encoding SEQ ID NO: 5) to reduce endogenous EHMT2/G9A levels (FIG. 2A). This virus also expresses GFP (FIG. 2B) and AAV-shG9A infusions produced a robust knockdown of endogenous EHMT2/G9A levels in the mouse NAc compared to the scrambled shRNA control (AAV-shSC; FIG. 2C; t(4)=2.856, p=0.046). Three weeks following bilateral infusions of AAV-shG9A or AAV-shSC, all mice were examined for baseline drinking in the 2-bottle choice test (FIG. 2D). Interestingly, EHMT2/G9A knockdown in NAc had no significant effects on baseline drinking (FIG. 2E; Three-Way ANOVA, Interaction: F_{3,90}=0.4136, p=0.7436), suggesting that reduction of EHMT2/G9A in NAc is not sufficient to increase or decrease ethanol drinking. As expected, CIE exposure escalated ethanol drinking in both virus groups (FIG. 2F; Three-Way ANOVA, F_{1,30}=24.96, p<0.0001), but surprisingly, there was no effect of NAc EHMT2/G9A knockdown on CIE-induced escalated drinking (F_{2,60}=0.1506, p=0.8605). As such, the observed reduction in NAc EHMT2/G9A protein levels following CIE treatment (see FIGS. 1B and 1C) did not appear to influence CIE-escalated drinking; however, since the EHMT2/G9A knockdown is in the same direction as the CIE effect on EHMT2/G9A levels, we cannot rule out the possibility that reduction of EHMT2/G9A levels is necessary, but not sufficient, for CIE-escalated ethanol drinking.

### Example 3

### EHMT2/G9A is Required for Stress-Potentiated Ethanol Drinking

EHMT2/G9A is required for stress-induced drug seeking in an extinction-reinstatement model of cocaine self-administration (Anderson et al., 2019), and similar to the effects of CIE, chronic cocaine exposure produces a reduction in NAc EHMT2/G9A and Histone H3K9me2 (Maze et al., 2010). In addition, stress is a major driver of heavy alcohol drinking and relapse in individuals suffering from AUDs (Brady & Sonne, 1999; Sinha, 2001; Spanagel et al., 2014). To examine the potential role of NAc EHMT2/G9A knockdown on stress-potentiated ethanol drinking, we extended the CIE/Air treatment for 2 additional rounds (5 total) before testing for stress-responsive drinking (FIG. 3A). All mice were then injected for 2 consecutive days with saline (i.p.) to habituate them to handling and injection stress, which did not alter the escalated drinking following CIE-exposure (FIG. 3B, Two-Way ANOVA, interaction: F_{1,30}=0.005401, p=0.9419; CIE vs Air: F_{1,30}=7.329, p=0.0111; virus: F_{1,30}=0.06250, p=0.8043). To stimulate stress-potentiated drinking, we treated mice with U50,488, a potent kappa opioid receptor agonist known to enhance ethanol drinking (Anderson et al., 2016a). A low dose of U50,488 (1.25 mg/kg; i.p.) had only modest effects on drinking. However, in the Air-treated and virus control group, a second exposure to a high dose of U50,488 (5 mg/kg; i.p.) produced a robust increase in ethanol drinking. However, stress-potentiated drinking was absent in the Air-treated/AAV-shG9A group, suggesting that NAc EHMT2/G9A is required for stress-potentiated drinking in non-dependent animals (FIG. 3C; Two-Way ANOVA, interaction: F_{1,14}=5.819, p=0.0302; drug: F_{1,14}=12.95, p=0.0029; virus: F_{1,14}=4.423, p=0.0540). In contrast, U50,488-treatment failed to increase drinking levels in the CIE-treated groups, possibly due to a ceiling effect of the higher ethanol intake levels produced by CIE (FIG. 3D; Two-Way ANOVA, interaction: F_{1,16}=0.3175, p=0.5809; drug: F_{1,16}=1.960, p=0.1806; virus: F_{1,16}=0.07293, p=0.7906). Plotted in a different way, the U50,488 injection increased the Air/shSC control animals to CIE-escalated levels, and NAc EHMT2/G9A is required for the stress-potentiated drinking in the non-dependent mice (FIG. 3E; Two-Way ANOVA, interaction: F_{1,30}=5.609, p=0.0245; CIE vs Air: F_{1,30}=1.783, p=0.1918; virus: F_{1,30}=12.00, p=0.0016). Taken together, our findings suggest that NAc-specific EHMT2/G9A is not required for baseline or CIE-escalated drinking, but NAc EHMT2/G9A is required for stress-potentiated drinking.

In mice, different stressors can have distinct effects on ethanol drinking (Becker et al., 2011; Anderson et al., 2016b; Lopez et al., 2016). To determine whether NAc EHMT2/G9A was required for different types of stress-regulated ethanol drinking, we compared the role of NAc EHMT2/G9A for U50,488-potentiated versus predator odor-suppressed ethanol drinking. Following bilateral virus infusions (AAV-shG9A or AAV-shSC), we established the baseline of ethanol drinking for 4 weeks (FIG. 4A). Similar to our previous results (FIG. 3B), the reduction of NAc EHMT2/G9A levels did not alter baseline ethanol drinking (FIG. 4B, Two-Way ANOVA, interaction: F_{3,63}=0.2184, p=0.8833; time: F_{3,63}=18.48, p<0.0001; virus: F_{1,21}=0.3883, p=0.5399). As expected (FIG. 3C), the injection of U50,488 (5 mg/kg; i.p.) in the virus control group (AAV-shSC) increased ethanol drinking, but it failed to increase ethanol drinking in the AAV-shG9A group (FIG. 4C, Two-Way ANOVA, interaction: F_{1,21}=4.517, p=0.0456; stress: F_{1,21}=3.519, p=0.0747; virus: F_{1,21}=3.261, p=0.0853), supporting our finding that NAc EHMT2/G9A was required for kappa opioid agonist-potentiated ethanol drinking. When the mice were exposed to a predator odor (soiled rat bedding), we observed a stress-induced suppression of ethanol drinking; however, reduction of NAc EHMT2/G9A again blocked this stress-induced effect (FIG. 4D, Two-Way ANOVA, interaction: F_{1,21}=7.034, p=0.0149; stress: F_{1,21}=16.93, p=0.0005; virus: F_{1,21}=1.619, p=0.2172). Both the potentiating (U50,488) and suppressing (predator odor) effects of these stressors on volitional ethanol drinking were reversible as shown by mice returning to baseline levels of drinking (FIG. 4E, Two-Way ANOVA, interaction: F_{1,21}=2.807, p=0.1087; stress: F_{1,21}=1.012, p=0.3259; virus: F_{1,21}=0.5164, p=0.4803). Together, these data suggested that EHMT2/G9A was required for multiple forms stress-regulated ethanol drinking.

### Example 4

Systemic EHMT2/G9A Inhibition Suppresses Stress-induced Ethanol Drinking Since reducing EHMT2/G9A levels in the NAc blocked stress-regulated ethanol drinking in mice, we tested whether systemic delivery of a specific EHMT2/G9A methyltransferase inhibitor, UNC0642 (Liu et al, 2013), could block stress-potentiated ethanol drinking. Wild-type mice were allowed to drink ethanol for 2 weeks prior to repeated, daily injections of UNC0642 (4 mg/kg; i.p.) given 30 minutes prior to the 2-bottle choice sessions (FIG. 5A). Interestingly, we observed a significant reduction in ethanol drinking during the first week of daily UNC0642 injections (FIG. 5B, week 3; Two-Way ANOVA, interaction: F_{3,63}=4.435, p=0.0068; drug: F_{1,21}=0.9138, p=0.3500; time: F_{3,63}=8.268, p=0.0001), but there was no difference in ethanol intake between vehicle control- and UNC0642-injected mice in the second week of daily drug treatments (FIG. 5B, week 4). Similar to the effects of NAc EHMT2/G9A knockdown above, chronic UNC0642 treatment suppressed U50,488-potentiated ethanol drinking observed in vehicle control mice (FIG. 5C, Two-Way ANOVA, interaction: F_{1,21}=6.595, p=0.0179; drug: F_{1,21}=1.481, p=0.2371; time: F_{1,21}=5.623, p<0.0001). Mice were then allowed to continue ethanol drinking for one additional week before the UNC0642-naïve mice (former vehicle control group) were injected with either vehicle or UNC0642 (4 mg/kg; i.p.) and tested for U50,488-potentiated ethanol drinking. Unlike chronic UNC0642 administration, the single injection of UNC0642 failed to influence stress-potentiated drinking (FIG. 5D; Two-Way ANOVA, interaction: F_{1,21}=0.9236, p=0.3470; drug: F_{1,21}=0.5679, p=0.4591; time: F_{1,21}=17.03, p=0.0004). These data suggest that chronic, but not acute, systemic EHMT2/G9A inhibition can block stress-potentiated drinking, and that pharmacological EHMT2/G9A inhibition is a viable candidate therapeutic for reducing stress-induced alcohol drinking.

### Example 5

### Systemic G9A Inhibition Suppresses Dependence-Induced Ethanol Drinking

Since systemic delivery of UNC0642 blocked stress-regulated ethanol drinking in mice, we tested whether systemic delivery of UNC0642 could block dependence-induced escalation of ethanol drinking also. As shown in FIG. 6A, wild-type mice were given 4 weeks of baseline drinking using a 1-hour limited access model (15% (v/v) ethanol vs. water, 2 h/d, 5d/wk, starting 3 hours into the dark phase of the circadian cycle). Mice were then divided into 4 evenly matched groups. Mice were exposed to Air/No stress, Air/FSS, CIE/No Stress, or CIE/stress to assess the effects of UNC0642 on ethanol drinking influenced by alcohol dependence alone or stress+dependence. Chronic injections of either vehicle or 4 mg/kg UNC0642 were started during the “test 4” phase illustrated in FIG. 6A and continued for 15 days. G9a inhibition caused a significant reduction in dependence-induced ethanol drinking during the “test 5” week (FIG. 6B, left panel, test 5; Two-Way ANOVA, interaction: F_{1,35}=20.23, p<0.0001; drug: F_{1,35}=8.392, p=0.0065; dependence: F_{1,35}=61.66, p<0.0001). Similar to the effects on dependence-induced escalated drinking alone, chronic UNC0642 treatment also suppressed stress+dependence ethanol drinking (FIG. 6B, right panel, test 5, Two-Way ANOVA, interaction: F_{1,34}=7.371, p=0.0103; drug: F_{1,34}=7.305, p=0.0107; time: F_{1,34}=30.86, p<0.0001). These data suggest that chronic G9A inhibition can suppress both dependence-escalated ethanol drinking and, stress+dependence potentiated alcohol drinking, and that pharmacological G9A inhibition is a viable candidate therapeutic for reducing both dependence-induced and stress-induced alcohol drinking.

### Example 6

### Systemic EHMT2/G9A Inhibition Suppresses Stress-Potentiated Alcohol Drinking and Reduces Anxiety-Like Behavior

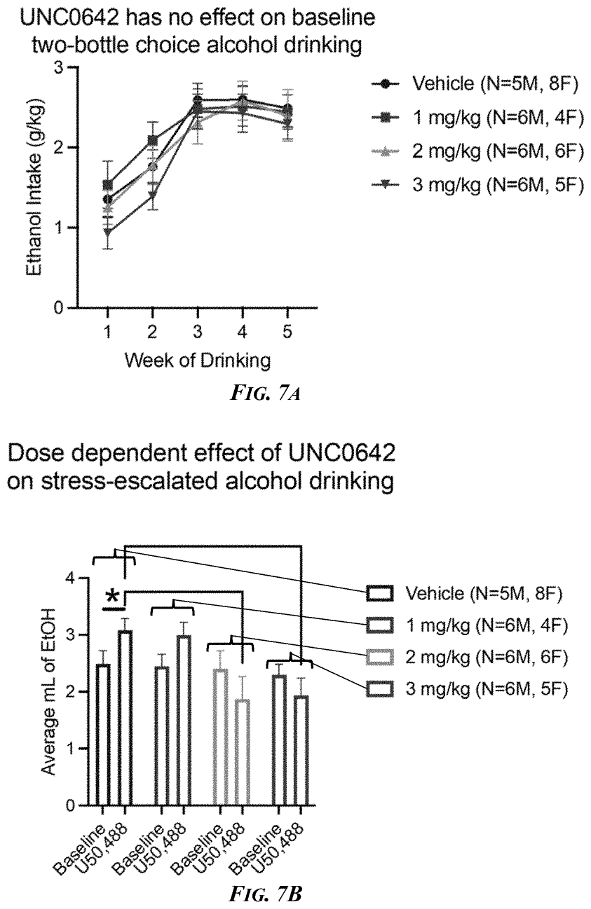

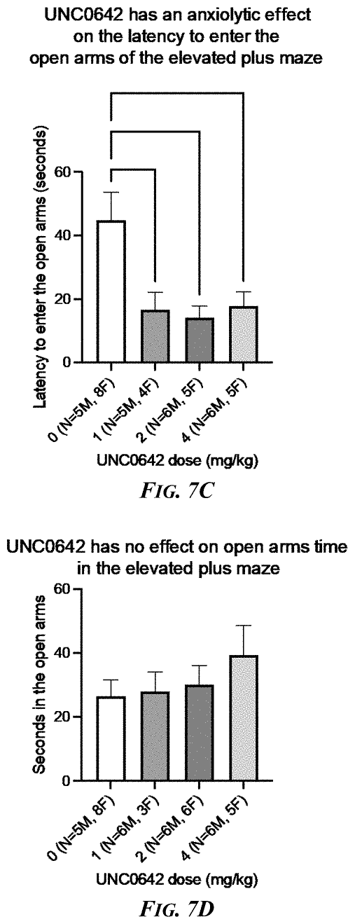

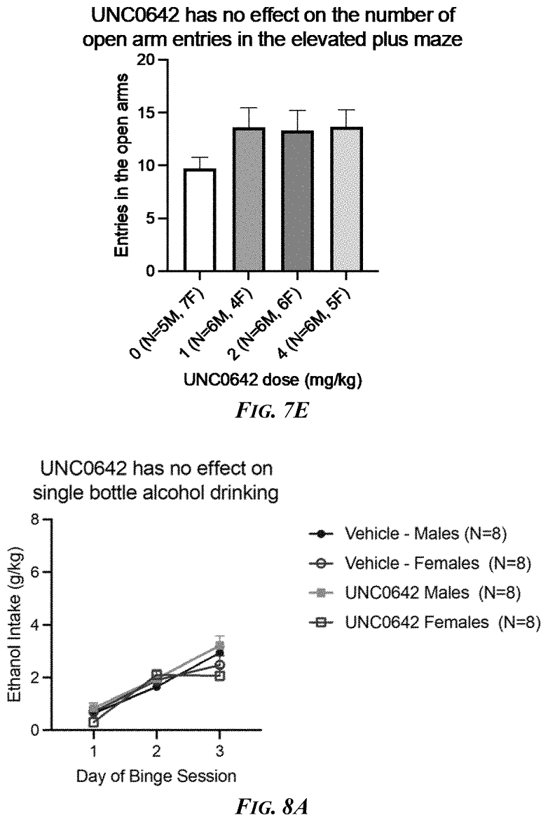

Since UNC0642 (4 mg/kg, i.p.) blocked stress-regulated ethanol drinking in mice as shown above, it was next tested whether this effect was dose-dependent and if it worked similarly in both sexes. There are reported sex/gender differences in the risk of developing several neuropsychiatric conditions, including AUD, SUD, and mood disorders, and while there was no a priori expectation of a sex-difference in UNC0642 efficacy in reducing stress-potentiated alcohol drinking, we wished to confirm that the treatment would be effective in both males and females. Similar to the studies described hereinabove (FIG. 5A), wild-type mice (both male and female) were allowed to drink ethanol for 2 weeks prior to repeated, daily injections of UNC0642 (4 mg/kg; 2 mg/kg, 1 mg/kg, or 0 mg/kg (vehicle), i.p.) given 30 minutes prior to the 2-bottle choice sessions. No changes were observed in alcohol drinking during the three weeks of daily UNC0642 injections as UNC0642 had no effect on baseline two-bottle choice alcohol drinking (weeks 3-5; FIG. 7A, Two-Way ANOVA, interaction: F12,168=0.5944, p=0.8447; drug: F3,42=0.6140, p=0.6098; time: F3,417,143.5=39.57, p<0.0001). Similar to the effects of reducing EHMT2/G9A levels in the NAc (i.e., shRNA knockdown), and the studies described above, chronic UNC0642 treatment suppressed U50,488-potentiated ethanol drinking observed in vehicle control mice at both the 4 mg/kg and 2 mg/kg doses. These data demonstrated a dose-dependent effect of UNC0642 on stress-escalated alcohol drinking (FIG. 7B, Two-Way ANOVA, interaction: F3,42=4.361, p=0.0092; drug: F3,42=2.435, p=0.0781; stress: F1,42=0.1755, p=0.6774). These data suggested that chronic, systemic EHMT2/G9A inhibition blocked stress-potentiated drinking at both 4 mg/kg and 2 mg/kg but not 1 mg/kg doses, and that pharmacological EHMT2/G9A inhibition is a viable candidate therapeutic for reducing stress-potentiated alcohol drinking at a lower dose than previously established. Importantly, this effect was observed in both male and female mice and UNC0642 reduces stress-potentiated alcohol drinking in both sexes similarly. So despite some sex/gender-related differences in AUD risk, patient proportions, and known differences in alcohol drinking behaviors in several preclinical AUD models, UNC0642 appeared to be similarly efficacious for both males and females, which increases its potential treatment utility for the general AUD patient population or related comorbid treatment contexts.

These same mice were tested on a 5-minute elevated plus maze protocol at 1-3 days after their final drinking session. 30-60 minutes following an i.p. injection of either 0, 1, 2, or 4 mg/kg UNC0642, mice were allowed to freely explore the open and closed arms of the elevated plus maze. Less anxious mice typically explore the open arms more quickly and spend more time exploring the open arms. Consistent with an anxiolytic response, all 3 doses of UNC0642 reduced the latency to explore the open arms of the elevated plus maze, meaning that UNC0642 had an anxiolytic effect on the latency to enter the open arms of the elevated plus maze (FIG. 7C, One-Way ANOVA, F3,41=5.772, p=0.0022, 1 outlier removed from the 1 mg/kg and 2 mg/kg groups each with a Grubbs test). Importantly, this effect was observed in both male and female mice and UNC0642 reduced anxiety in both sexes similarly, which suggested that, similar to effects of UNC0642 on stress-regulated alcohol drinking, the anxiolytic mechanisms regulated by G9a and UNC0642, were independent of sex/gender-based influences. No statistically significant effects were observed on the time spent in the open arms, as UNC0642 had no effect on open arms time in the elevated plus maze (see FIG. 7D; One-Way ANOVA, F3,42=0.7372, p=0.5359, 1 outlier removed from the 1 mg/kg group with a Grubbs test) or on the number of entries into the open arms, meaning that UNC0642 had no effect on the number of open arm entries in the elevated plus maze (FIG. 7E; One-Way ANOVA, F3,42=1.396, p=0.2573, 1 outlier removed from the 0 mg/kg group with a Grubbs test). Taken together, these results suggested that UNC0642 has anxiolytic properties in addition to the ability to reduce stress- and dependence-potentiated alcohol drinking. Thus, in some embodiments, it can be particularly useful for treating individuals with co-morbid psychiatric diseases and/or disorders and AUD/SUD.

These results also suggested that G9a inhibition can be useful in treating symptoms in individuals diagnosed with stress- and/or anxiety-related disorders and/or disorders exacerbated by stress and anxiety, including post-traumatic stress disorder (PTSD), panic disorder, social anxiety disorder, general anxiety disorder, and major depressive disorder, as these disorders are often co-morbid with AUD and/or SUD (Moss et al, 2010; Back & Brady, 2008; Vorspan et al, 2015; Hunt et al., 2020; Kaysen et al., 2014; Schneier et al., 2010; Lespine et al., 2022; Torvik et al., 2019).

### Example 7

### Systemic EHMT2/G9A Inhibition does not Alter Binge-Like Ethanol Drinking

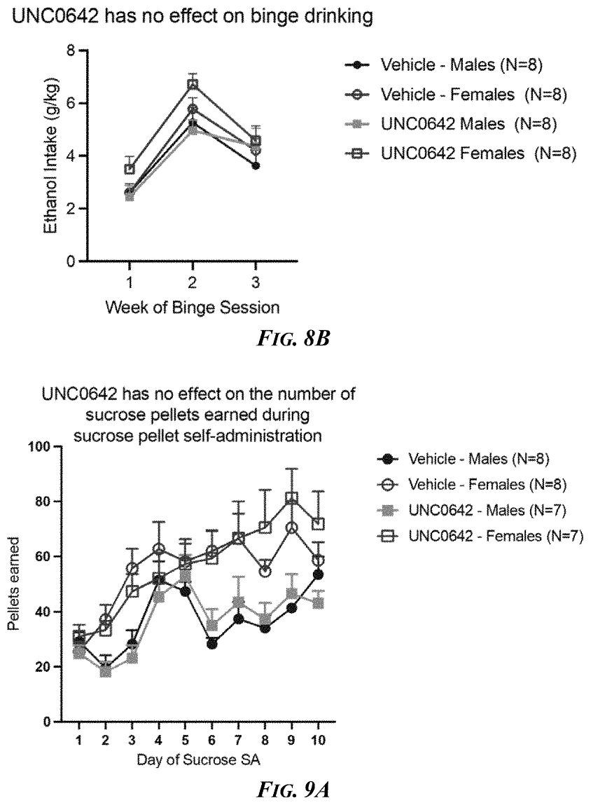

Since UNC0642 blocked stress-potentiated ethanol drinking in mice, as show above, we next tested whether UNC0642 could alter binge-like ethanol drinking in both sexes. Wild-type mice (both male and female) were given ethanol for 1 week in a “Drinking in the Dark” paradigm (Rhodes et al., 2005), a well-established alcohol model that is similar to binge-drinking in humans. Both male and female mice were given a single bottle of 20% ethanol in their home cage for 2 hours per day for 3 days. No alternative bottle of water was given in this experiment. On the 4th day, the access to the bottle was extended to 4 hours to model a binge-drinking session. After the first week, mice were split into even groups and were injected with UNC0642 (2 mg/kg, i.p.) or vehicle for 10 days 30 minutes before each drinking session began. Mice were tested for changes in baseline drinking and binge drinking for 2 weeks. We observed no changes in alcohol drinking during the baseline 2 hour sessions, meaning that UNC0642 had no effect on single-bottle alcohol drinking (FIG. 8A; Three-Way ANOVA, Time: F1.996,55.88=47.99, p<0.0001, Sex: F1,28=4.229, p=0.0492, no other significant effects) nor the 4-hour binge sessions, meaning that UNC0642 had no effect on binge alcohol drinking (FIG. 8B; Three-Way ANOVA, Time: F1.909,53.45=78.43, p<0.0001, Day X Sex: F2,56=4.552, p=0.0147, no other significant effects). Our data detected the well-documented sex differences in alcohol intake in these assays, but there was no significant interaction between sex and UNC0642 treatment. These data suggested that UNC0642 did not alter binge alcohol drinking in mice, at least at the 2 mg/kg UNC0642 dose that effectively suppressed stress-potentiated alcohol drinking as shown in EXAMPLE 6. These results also re-confirmed that UNC0642 did not alter baseline alcohol drinking as observed in other experiments set forth hereinabove.

### Example 8

### Systemic EHMT2/G9A Inhibition does not Alter Sucrose Self-Administration Behavior

Since the effects of UNC0642 on stress- and dependence-potentiated alcohol drinking could be influenced by a reduction of general reward-seeking behavior, it was next tested the effects of UNC0642 on self-administration of sucrose.

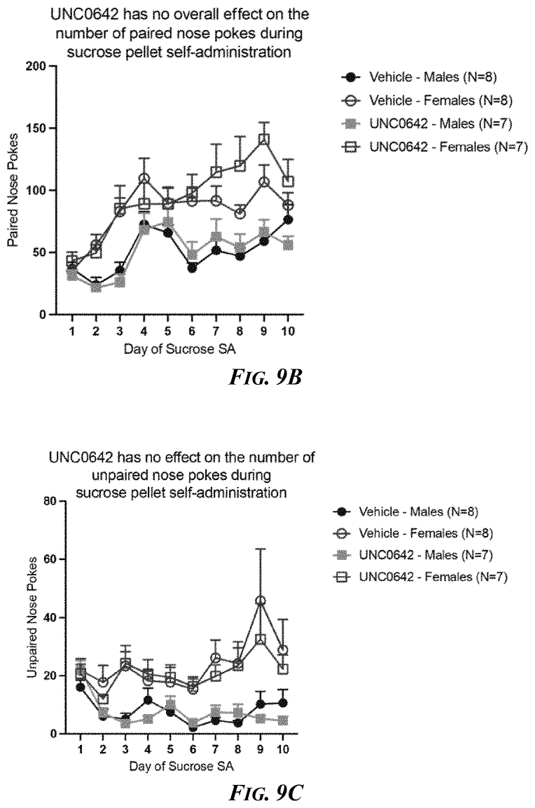

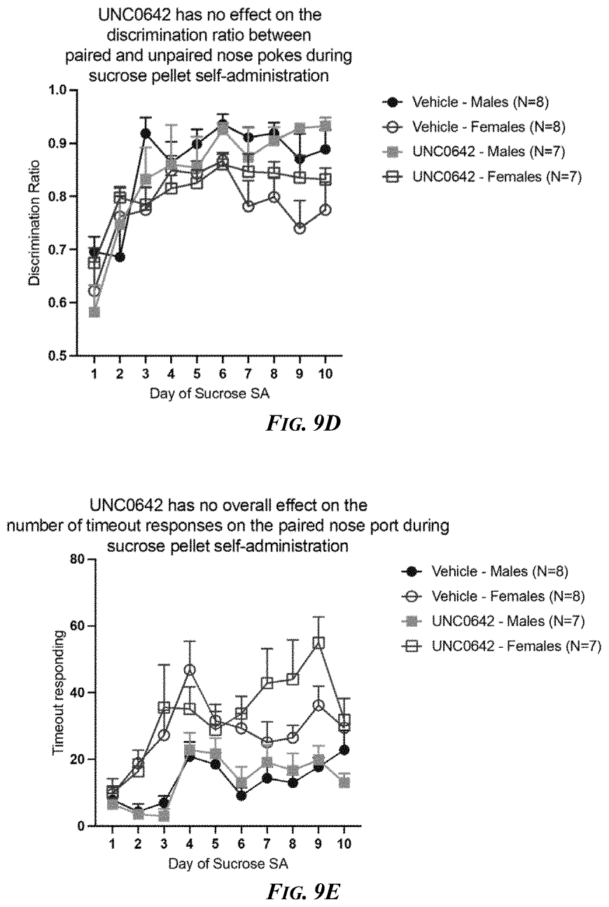

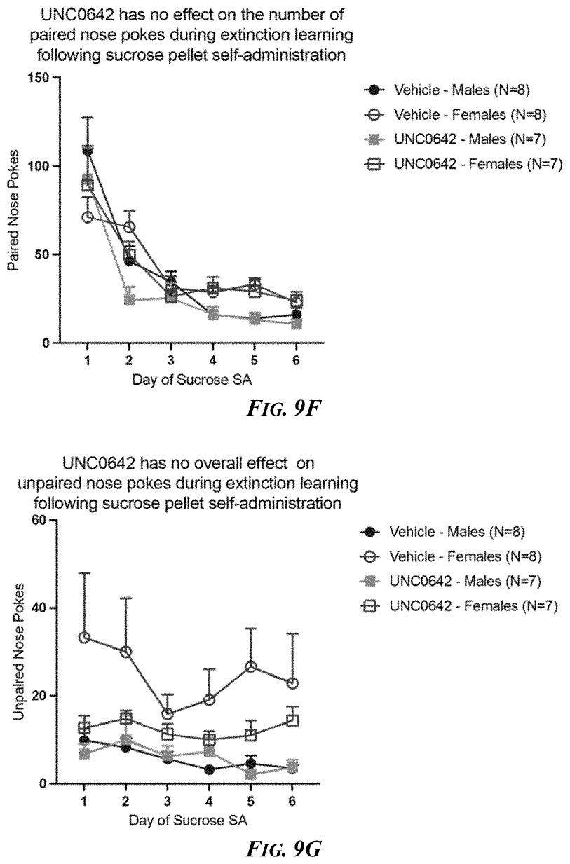

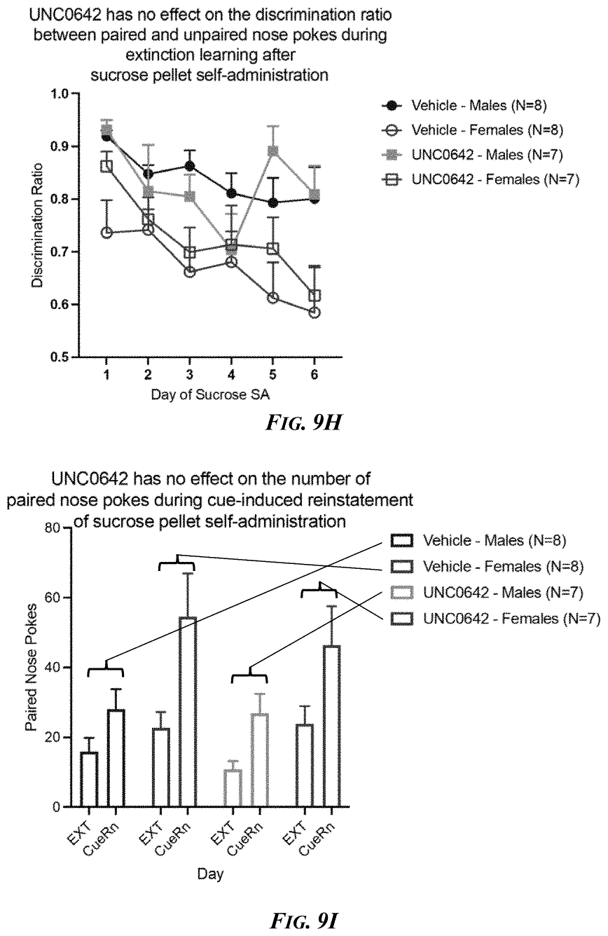

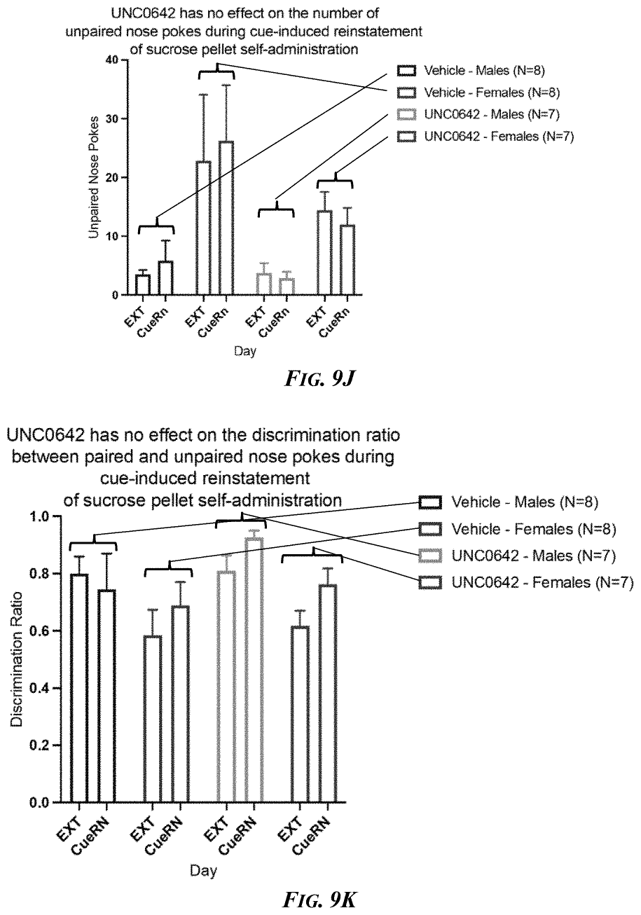

Mice were first injected with UNC0642 (2 mg/kg, i.p) or vehicle-alone once daily for 10 days. Thirty minutes following the 10th dose, they began the first 2-hour sucrose self-administration session. The UNC0642 and vehicle injections occurred 30 minutes before the start of each sucrose self-administration, extinction, and reinstatement session. All self-administration experiments occurred in standard operant chambers with two nose-poke detectors, a house light, and a cue light and tone generator (Med Associates, Fairfield, Vt.). All sucrose-paired nose-pokes, including those during the timeout, were recorded and are reported as “paired nose pokes”. No fasting occurred during any stage of the experiment. During the 2-hour sessions, mice were trained to nose-poke in the paired side on a fixed-ratio 1 (FR1) schedule with 15-second timeout period after delivery of a single sucrose pellet. Concurrent with the pellet delivery, a cue tone and cue light (which is located immediately above the paired nose-poke port) are activated. All mice had ten self-administration sessions. No significant differences were detected in the number of sucrose pellets earned over the course of the experiment, meaning that UNC0642 had no effect on the number of sucrose pellets earned during sucrose pellet self-administration (FIG. 9A; Three-Way ANOVA, Day: F1.885,50.90=22.80, p<0.0001, Sex: F1,27=11.41, p=0.0022, Day X Sex: F9,243=4.535, p<0.0001, no other significant effects. While there was no overall effect of UNC0642 on paired nose-pokes there was a significant interaction between the self-administration day and the UNC0642 treatment, suggesting that female mice had a slight increase in paired nose-pokes during self-administration in the UNC0642 group, as UNC0642 had no overall effect on the number of paired nose pokes during sucrose pellet self-administration (FIG. 9B; Three-Way ANOVA, Day: F2.222,57.78=23.20, p<0.0001, Sex: F1,26=20.03, p=0.0001, Day X Sex: F9,234=4.721, p<0.0001, Day X Treatment: F9,234=2.060, p=0.0339, no other significant effects, including no effect on Treatment X Sex, F1,26=0.3544, p=0.5568). No differences were observed in inactive nose-pokes, as UNC0642 had no effect on the number of unpaired nose pokes during sucrose pellet self-administration (FIG. 9C; Three-Way ANOVA, Day: F2.320,60.31=3.272, p=0.0380, Sex: F1,26=21.90, p<0.0001, Day X Sex: F9,234=3.018, p=0.0020, no other significant effects) or in the discrimination ratio of the paired and unpaired nose-pokes, as UNC0642 had no effect on the discrimination ratio between paired and unpaired nose pokes during sucrose pellet self-administration (FIG. 9D; Three-Way ANOVA, Day: F3.496,90.90=15.98, p<0.0001, Sex: F1,26=5.146, p=0.0318, Day X Sex: F9,234=2.315, p=0.0164, no other significant effects, including no effect on Treatment X Sex, F1,26=0.8646, p=0.3610). Finally, we examined the number of nose-pokes on the paired side during timeout periods. Similar to the paired nose-poke data above, there was a slight effect on the interaction between UNC0642 treatment and sex. Female mice in the UNC0642 group had slightly higher timeout responding, but UNC0642 had no overall effect on the number of timeout responses on the paired nose port during sucrose pellet self-administration (FIG. 9E; Three-Way ANOVA, Day: F3.085,80.21=12.81, p<0.0001, Sex: F1,26=24.29, p<0.0001, Day X Sex: F9,234=2.889, p=0.0029, Day X Treatment: F9,234=1.929, p=0.0488, no other significant effects, including no effect on Treatment X Sex, F1,26=0.4990, p=0.4862). Since there appears to be only a very subtle effect on active lever pressing in female mice and no effect in male mice, this suggests that UNC0642's effects likely cannot be attributed to a deficit in learning and memory or an effect on general reward-taking behavior. Overall, UNC0642 had no effect on sucrose-taking behavior as all groups increased their responding for sucrose pellets over time, and they learned and remembered the relationship between the reinforced (sucrose-paired) and the non-reinforced (unpaired) ports.

Sucrose-seeking behaviors were next tested in these same mice. Following a 7-day period in the home cage with ad libitum food and water, all mice began extinction training for 6 consecutive days (a model of context-associated sucrose-seeking behavior). During these 2-hour sessions, nose-pokes in either port resulted in no sucrose pellet delivery nor presentation of the light and tone cues. No differences were observed in paired nose-pokes, as UNC0642 had no effect on the number of paired nose pokes during extinction learning following sucrose pellet self-administration (FIG. 9F; Three-Way ANOVA, Day: F1.560,40.55=49.19, p<0.0001, Day X Sex: F5,130=4.052, p=0.0019, no other significant effects), unpaired nose-pokes during extinction learning following sucrose pellet self-administration (FIG. 9G; Three-Way ANOVA, Sex: F1,26=6.976, p=0.0138, no other significant effects), or the discrimination ratio between paired and unpaired nose pokes during the extinction learning after sucrose pellet self-administration (FIG. 9H; Three-Way ANOVA, Day: F3.741,97.27=7.902, p<0.0001, Sex: F1,26=11.46, p=0.0023, no other significant effects). Finally, light/tone cue-induced reinstatement behavior (a model of discreet cue-induced sucrose-seeking behavior) was tested one day after the 6th extinction session in a single 2-hour session. Mice were allowed to nose-poke for light and tone cues without receiving pellets. During the cue test, a significant effect of reinstatement was observed, suggesting that all groups reinstated to cues. However, we found no differences in paired nose-pokes between groups, suggesting no effect of UNC0642 on the number of paired nose pokes during cue induced reinstatement of sucrose pellet self-administration (FIG. 9I; Three-Way ANOVA, Reinstatement: F1,26=26.79, p<0.0001, Sex: F1,26=7.481, p=0.0111, no other significant effects). We also found UNC0642 had no effect on UNC0642 on the number of unpaired nose pokes during cue induced reinstatement of sucrose pellet self-administration (FIG. 9J; Three-Way ANOVA, Day: F1,26=6.739, p=0.0153, Sex: F1,26=6.739, p=0.0153, no other significant effects) and UNC0642 had no effect on the discrimination ratio between paired and unpaired nose pokes during cue-induced reinstatement of sucrose pellet self-administration (FIG. 9K; Three-Way ANOVA, Sex: F1,26=5.880, p=0.0226, no other significant effects).

Overall, no statistically significant effects of UNC0642 on the acquisition or stable intake of sucrose self-administration, extinction of sucrose seeking, or cue-induced reinstatement of sucrose seeking were detected. These results suggest that UNC0642's effects are selective to stress-potentiated and dependence-escalated alcohol drinking and are independent of sex.

### Discussion of the Examples

In these studies, we discovered that chronic ethanol exposure reduced NAc levels of EHMT2/G9A protein and histone H3K9me2, its well-documented enzymatic target (FIG. 1B). Also, CIE-exposed, but not Air-exposed, mice showed a negative correlation between NAc EHMT2/G9A mRNA and ethanol drinking levels (FIGS. 1E and 1F), but we observed no evidence that viral-mediated reduction of NAc EHMT2/G9A levels altered baseline or CIE-escalated ethanol drinking levels. However, either viral-mediated reduction of EHMT2/G9A in NAc or systemic inhibition of EHMT2/G9A activity blocked stress-regulated ethanol drinking in mice. Together, our findings suggested that the reduction of NAc EHMT2/G9A following repeated ethanol exposure limited stress-regulated ethanol drinking although other brain areas like the amygdala could also play a role (Berkel et al., 2019). As stress is a major trigger for heavy alcohol drinking and relapse in individuals suffering from AUD, our findings suggest that EHMT2/G9A inhibition could be a viable therapeutic strategy to reduce the vulnerabilities to stress-induced heavy alcohol drinking and/or relapse. Since other abused substances, like cocaine and opioids, also reduce NAc EHMT2/G9A levels (Maze et al., 2010; Sun et al., 2012) and limit stress-induced reinstatement of drug seeking (Anderson et al., 2019), inhibition of EHMT2/G9A activity might be an effective treatment strategy in humans to limit relapse vulnerability in polysubstance abusers.

Since NAc EHMT2/G9A mRNA levels in CIE-treated mice correlated negatively with ethanol drinking, it was unexpected that viral-mediated reduction of NAc EHMT2/G9A had no apparent impact on levels of alcohol drinking in ethanol-dependent (or non-dependent) mice. This suggested that neither the reinforcing effects of alcohol nor the mechanisms underlying CIE-induced escalation of ethanol drinking were dependent on NAc EHMT2/G9A levels or activity. However, NAc EHMT2/G9A did regulate stress-reactive ethanol drinking behavior (see FIGS. 3C, 4C, and 4D). While alcohol dependence can increase stress reactivity (Liu & Weiss, 2002; Becker et al., 2011; Anderson et al., 2016b; Lopez et al., 2016), the presently disclosed findings suggested that the CIE-enhanced ethanol drinking was independent of NAc EHMT2/G9A's role in stress modulation.

As a key regulator of chromatin landscape and nuclear gene expression, we assume that EHMT2/G9A modulates stress-regulated ethanol drinking via an epigenetic mechanism. EHMT2/G9A-mediated dimethylation of histone H3K9me2 is typically associated with gene repression (Anderson et al., 2018a), and reduction of EHMT2/G9A (and H3K9me2) would likely increase gene expression of many target genes that ultimately suppress stress-reactivity. Prior studies have reported hundreds of genes that are differentially expressed in the absence or overexpression of EHMT2/G9A (Maze et al., 2010; Maze et al., 2014), and it is possible that multiple dysregulated NAc gene targets combine to regulate stress reactive drinking. As such, future studies exploring the relevant gene target(s) can enhance understanding of the precise molecular and cellular mechanisms underlying NAc EHMT2/G9A's role in stress-reactive drug and alcohol taking and seeking behaviors.

In the present study, two distinct models of stress-regulated ethanol drinking were employed, and viral-mediated reduction of NAc EHMT2/G9A blocked stress-regulated ethanol drinking in both (see FIGS. 3C, 4C, and 4D). Systemic activation of the kappa opioid receptor (KOR) using the agonist U50,488 promoted increased ethanol drinking, as previously reported (Anderson et al., 2016a). KORs are activated by numerous stressful stimuli and KOR activation is sufficient to produce aversive and dysphoric states (Bals-Kubik et al., 1993; Mague et al., 2003; Todtenkopf et al., 2004; Carlezon et al., 2006). In addition, the KOR system is dysregulated in AUD/SUD (Anderson and Becker, 2017) and is a major contributor to the high comorbidity of addiction and depression (Bruchas et al., 2010; Crowley & Kash, 2015). It was found that viral-mediated reduction of NAc EHMT2/G9A blocked the KOR-potentiated ethanol drinking, but without altering baseline ethanol drinking (see FIGS. 2E, 3B-3E, and 4B-4E.) How EHMT2/G9A reduction blocks U50,488-potentiated drinking is unclear, but it is possible that EHMT2/G9A supports KOR signaling within the NAc. Both EHMT2/G9A and KOR (and its endogenous agonist prodynorphin) are expressed in the NAc (Przewlocka et al., 1997), and NAc Shell-specific injection of a KOR antagonist reduced ethanol drinking in alcohol-preferring rats (Uhari-Vaananen et al., 2018) and in ethanol-dependent self-administering animals (Nealey et al., 2011).

The second stress-related model used was predator odor exposure. Mice were exposed to dirty rat bedding using a protocol that in the past led to stress-induced increases in drinking (Cozzoli et al., 2014). However, as disclosed herein, control mice exposed to predator odor unexpectedly decreased ethanol drinking. However, this stress-induced effect was blocked by viral-mediated reduction of NAc EHMT2/G9A (FIG. 4D), suggesting that EHMT2/G9A was mediating stress reactivity, regardless of the behavioral outcome of the stress exposure. In rodents, stress and alcohol interactions can either increase or decrease ethanol consumption, depending on the experimental conditions (Becker et al., 2011; Anderson et al., 2016b; Lopez et al., 2016), and the presently disclosed findings suggested that NAc EHMT2/G9A was required for changes in ethanol drinking produced by multiple types of stress exposure.

In sum, the present findings demonstrated that NAc EHMT2/G9A was required for stress-regulated drinking in both ethanol-dependent and non-dependent animals. In contrast, NAc EHMT2/G9A did not play an obvious role in volitional drinking or CIE-induced escalation of drinking. Interestingly though, systemic administration of a EHMT2/G9A inhibitor reduced both U50,488-stress-induced ethanol drinking and dependence-induced drinking, suggesting that EHMT2/G9A inhibition was more effective in reducing EHMT2/G9A activity in the NAc than the AAV-shG9a virus. These findings also suggested that chronic ethanol exposure produced reductions in NAc EHMT2/G9A and histone H3K9me2 that appeared to function as a counter-adaptations to limit future stress reactivity. Also, additional studies using systemic administration of a EHMT2/G9A inhibitor dose-dependently reduced both U50,488-stress-induced ethanol drinking and anxiety-like behavior in both male and female mice. These effects were unlikely due to an effect of EHMT2/G9a inhibition on rewarding behaviors, or mechanisms of learning and memory since no effects on sucrose-taking, sucrose-seeking, non-potentiated ethanol drinking, or binge drinking were observed. These results suggested that the effects of systemically administered EHMT2/G9A inhibition were selective to stress-potentiated and dependence-escalated alcohol drinking and despite clear and well-documented sex differences in alcohol behavior in preclinical and clinical studies, the effects of EMHT2/G9A inhibition on stress- and dependence-potentiated alcohol drinking were independent of sex. Since the stress system is dysregulated in chronic substance abusers (Becker, 2012), pharmacological inhibition of EHMT2/G9A activity could prove to be a useful therapeutic strategy to treat relapse vulnerability in individuals suffering from AUD and SUD. Pharmacological inhibition of EHMT2/G9A activity can also especially help individuals with both SUD (e.g., AUD) and co-morbid psychiatric diseases and/or disorders (Moss et al, 2010; Back & Brady, 2008; Vorspan et al, 2015; Hunt et al., 2020; Kaysen et al., 2014; Schneier et al., 2010; Lespine et al., 2022; Torvik et al., 2019).

### REFERENCES

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Anderson & Becker (2017) Role of the Dynorphin/Kappa Opioid Receptor System in the Motivational Effects of Ethanol. Alcohol Clin Exp Res 41:1402-1418.
Anderson et al. (2016a) Stress-Induced Enhancement of Ethanol Intake in C57BL/6J Mice with a History of Chronic Ethanol Exposure: Involvement of Kappa Opioid Receptors. Front Cell Neurosci 10:45.
Anderson et al. (2016b) Forced swim stress increases ethanol consumption in C57BL/6J mice with a history of chronic intermittent ethanol exposure. Psychopharmacology 233:2035-2043.
Anderson et al. (2018a) It's a complex issue: Emerging connections between epigenetic regulators in drug addiction. European Journal of Neuroscience 50:2477-2491.
Anderson et al. (2018b) Overexpression of the Histone Dimethyltransferase G9A in Nucleus Accumbens Shell Increases Cocaine Self-Administration, Stress-Induced Reinstatement, and Anxiety. The Journal of neuroscience: the official journal of the Society for Neuroscience 38:803-813.
Anderson et al. (2019) Knockdown of the histone di-methyltransferase G9A in nucleus accumbens shell decreases cocaine self-administration, stress-induced reinstatement, and anxiety. Neuropsychopharmacology: official publication of the American College of Neuropsychopharmacology 44:1370-1376.
Badanich et al. (2011) Effects of chronic intermittent ethanol exposure on orbitofrontal and medial prefrontal cortex-dependent behaviors in mice. Behav Neurosci 125:879-891.
Back, S. E. & Brady, K. T. Anxiety Disorders with Comorbid Substance Use Disorders: Diagnostic and Treatment Considerations. Psychiatr Ann 38, 724-729, doi:10.3928/00485713-20081101-01(2008).
Bals-Kubik et al. (1993) Neuroanatomical sites mediating the motivational effects of opioids as mapped by the conditioned place preference paradigm in rats. The Journal of pharmacology and experimental therapeutics 264:489-495.
Beaucage et al. (eds.) (2002) Current Protocols in Nucleic Acid Chemistry, John Wiley & Sons, Inc., New York, N.Y., United States of America.
Becker (2012) Effects of alcohol dependence and withdrawal on stress responsiveness and alcohol consumption. Alcohol Res 34:448-458.
Becker & Lopez (2004) Increased ethanol drinking after repeated chronic ethanol exposure and withdrawal experience in C57BL/6 mice. Alcohol Clin Exp Res 28:1829-1838.
Becker et al. (2011) Effects of stress on alcohol drinking: a review of animal studies. Psychopharmacology 218:131-156.
Berkel et al. (2019) Essential Role of Histone Methyltransferase G9A in Rapid Tolerance to the Anxiolytic Effects of Ethanol. Int J Neuropsychopharmacol 22:292-302.
Brady & Sonne (1999) The role of stress in alcohol use, alcoholism treatment, and relapse. Alcohol Res Health 23:263-271.
Bruchas et al. (2010) The dynorphin/kappa opioid system as a modulator of stress-induced and pro-addictive behaviors. Brain Res 1314:44-55.
Carlezon et al. (2006) Depressive-like effects of the kappa-opioid receptor agonist salvinorin A on behavior and neurochemistry in rats. The Journal of pharmacology and experimental therapeutics 316:440-447.
Covington et al. (2011) A role for repressive histone methylation in cocaine-induced vulnerability to stress. Neuron 71:656-670.
Cozzoli et al. (2014) Environmental stressors influence limited-access ethanol consumption by C57BL/6J mice in a sex-dependent manner. Alcohol 48:741-754.
Crooke et al. (1996) Pharmacokinetic properties of several novel oligonucleotide analogs in mice. J Pharmacol Exp Ther 277:923-937.
Crowley & Kash (2015) Kappa opioid receptor signaling in the brain: Circuitry and implications for treatment. Prog Neuropsychopharmacol Biol Psychiatry 62:51-60. den Hartog et al. (2016) Inactivation of the lateral orbitofrontal cortex increases drinking in ethanol-dependent but not non-dependent mice. Neuropharmacology 107:451-459.
Elmen et al., (2005) Locked nucleic acid (LNA) mediated improvements in siRNA stability and functionality. Nucleic Acids Research 33(1):439-447.
Englisch et al. (1991) Chemically modified oligonucleotides as probes and inhibitors. Angewandte Chemie, International Edition 30:613.
Gangisetty et al. (2015) Fetal Alcohol Exposure Reduces Dopamine Receptor D2 and Increases Pituitary Weight and Prolactin Production via Epigenetic Mechanisms. PLoS One 10:e0140699.
GENBANK® Accession Nos. NC_000006.12, NM_006709.5, NM_025256.7, NM_145830.3, NM_212463.1, NM_001206263.2, NM_001286573.2, NM_001289413.1, NM_001318833.1, NM_001363689.1, NP_006700.3, NP_079532.5, NP_001276342.1, NP_001305762.1, NP_001350618.1, XM_023624646.1, XP_006715037.1, XP_006715038.1, XP_006715039.1, XP_016865691.1.
Griffin et al. (2009) Intensity and duration of chronic ethanol exposure is critical for subsequent escalation of voluntary ethanol drinking in mice. Alcohol Clin Exp Res 33:1893-1900.
Grunweller et al., (2003) Comparison of different antisense strategies in mammalian cells using locked nucleic acids, 2′-O-methyl RNA, phosphorothioates and small interfering RNA. Nucleic Acids Research 31(12):3185-3193.
Herdewijn (ed.) (2008) Modified nucleosides: in biochemistry, biotechnology and medicine. Wiley-VCH Verlag GmbH & Co., Weinheim, Germany.
Hunt et al., (2020) Prevalence of comorbid substance use in major depressive disorder in community and clinical settings, 1990-2019: Systematic review and meta-analysis. J Affect Disord. 266:288-304.
Kabanov et al. (1990) A new class of antivirals: antisense oligonucleotides combined with a hydrophobic substituent effectively inhibit influenza virus reproduction and synthesis of virus-specific proteins in MDCK cells. FEBS Lett 259:327-330.
Kaysen et al. (2014) Proximal relationships between PTSD and drinking behavior. Eur J Psychotraumatol. 5:26518.
Kroschwitz (ed.) (1990) The Concise Encyclopedia Of Polymer Science And Engineering John Wiley & Sons, New York, N.Y., United States of America, pages 858-859.
Lespine et al. (2022) Gender-related associations between psychiatric disorders and alcohol use disorder: Findings from the French “Mental health in the general population” survey. Arch Womens Ment Health. 25(5):895-902.
Letsinger et al. (1989) Cholesteryl-conjugated oligonucleotides: synthesis, properties, and activity as inhibitors of replication of human immunodeficiency virus in cell culture. Proc Natl Acid Sci USA 86:6553-6556.
Liu & Weiss (2002) Additive effect of stress and drug cues on reinstatement of ethanol seeking: exacerbation by history of dependence and role of concurrent activation of corticotropin-releasing factor and opioid mechanisms. The Journal of neuroscience: the official journal of the Society for Neuroscience 22:7856-7861.
Liu et al. (2013) Discovery of an in Vivo Chemical Probe of the Lysine Methyltransferases G9a and GLP. Journal of Medicinal Chemistry 56(21):8931-8942.
Lopez & Becker (2005) Effect of pattern and number of chronic ethanol exposures on subsequent voluntary ethanol intake in C57BL/6J mice. Psychopharmacology 181:688-696.
Lopez et al. (2016) Effect of different stressors on voluntary ethanol intake in ethanol-dependent and nondependent C57BL/6J mice. Alcohol 51:17-23.
Lopez et al. (2017) Variable effects of chronic intermittent ethanol exposure on ethanol drinking in a genetically diverse mouse cohort. Alcohol 58:73-82.
Mague et al. (2003) Antidepressant-like effects of kappa-opioid receptor antagonists in the forced swim test in rats. The Journal of pharmacology and experimental therapeutics 305:323-330.
Manoharan et al. (1992) Chemical modifications to improve uptake and bioavailability of antisense oligonucleotides. Ann NY Acad Sci 660:306-309.
Manoharan et al. (1993) Introduction of a lipophilic thioether tether in the minor groove of nucleic acids for antisense applications. Biorg Med Chem Lett 3:2765-2770.
Manoharan et al. (1994) Cholic acid-oligonucleotide conjugates for antisense applications. Biorg Med Chem Lett 4:1053-1060.
Manoharan et al. (1995a) Lipidic nucleic acids. Tetrahedron Lett 36:3651-3654.
Manoharan et al. (1995b) Oligonucleotide conjugates: alteration of the pharmacokinetic properties of antisense agents. Nucleosides & Nucleotides 14:969-973.
Martin et al. (1995) Ein neuer Zugang zu 2′-O-Alkylribonucleosiden und Eigenschaften deren Oligonucleotide. Helv Chim Acta 78:486-504.
Maze et al. (2010) Essential role of the histone methyltransferase G9A in cocaine-induced plasticity. Science 327:213-216.
Maze et al. (2014) G9A influences neuronal subtype specification in striatum. Nature neuroscience 17:533-539.
Mishra et al. (1995) Improved leishmanicidal effect of phosphorotioate antisense oligonucleotides by LDL-mediated delivery. Biochim Biophys Acta 1264:229-237.
Mook et al. (2007) Evaluation of locked nucleic acid-modified small interfering RNA in vitro and in vivo. Mol Canc Ther 6(3):833-843.
Moss et al. (2010) Prospective follow-up of empirically derived Alcohol Dependence subtypes in wave 2 of the National Epidemiologic Survey on Alcohol And Related Conditions (NESARC): recovery status, alcohol use disorders and diagnostic criteria, alcohol consumption behavior, health status, and treatment seeking. Alcohol Clin Exp Res 34, 1073-1083,
Nealey et al. (2011) kappa-opioid receptors are implicated in the increased potency of intra-accumbens nalmefene in ethanol-dependent rats. Neuropharmacology 61:35-42.
Nielsen et al. (1991) Sequence-selective recognition of DNA by strand displacement with a thymine-substituted polyamide. Science 254:1497-1500.
Oberhauser et al. (1992) Effective incorporation of 2-O-methyl-oligoribonuclectides into liposomes and enhanced cell association through modification with thiocholesterol. Nucl Acids Res 20:533-538.
PCT International Patent Application Publication Nos. WO 1992/02190 and WO 1993/16185.
Przewlocka et al. (1997) Ethanol withdrawal enhances the prodynorphin system activity in the rat nucleus accumbens. Neuroscience letters 238:13-16.
Qiang et al. (2011) Histone H3K9 modifications are a local chromatin event involved in ethanol-induced neuroadaptation of the NR2B gene. Epigenetics 6:1095-1104.
Rhodes et al. (2005) Evaluation of a simple model of ethanol drinking to intoxication in C57BL/6J mice. Physiol Behav. 84:53-63,
Rinker et al. (2017) Differential potassium channel gene regulation in BXD mice reveals novel targets for pharmacogenetic therapies to reduce heavy alcohol drinking. Alcohol 58:33-45.
Saison-Behmoaras et al. (1991) Short modified antisense oligonucleotides directed against Ha-ras point mutation induce selective cleavage of the mRNA and inhibit T24 cells proliferation. EMBO J 10:1111-1118.
Sanghvi (1993) in Antisense Research and Applications (Crooke & Lebleu (eds.)) CRC Press, Boca Raton, Fla., United States of America, pages 276-278 and 289-302.
Schneider et al. (2012). NIH Image to ImageJ: 25 years of image analysis. Nature Methods 9:671-675.
Schneier et al. (2020) Social anxiety disorder and alcohol use disorder co-morbidity in the National Epidemiologic Survey on Alcohol and Related Conditions. Psychol Med. 40(6):977-88.
Shea et al. (1990) Synthesis, hybridization properties and antiviral activity of lipid-oligodeoxynucleotide conjugates. Nucl Acids Res 18:3777-3783.
Sinha (2001) How does stress increase risk of drug abuse and relapse? Psychopharmacology 158:343-359.
Spanagel et al. (2014) Stress and alcohol interactions: animal studies and clinical significance. Trends in neurosciences 37:219-227.
Subbanna & Basavarajappa (2014) Pre-administration of G9A/GLP inhibitor during synaptogenesis prevents postnatal ethanol-induced LTP deficits and neurobehavioral abnormalities in adult mice. Exp Neurol 261:34-43.
Subbanna et al. (2013) G9A-mediated histone methylation regulates ethanol-induced neurodegeneration in the neonatal mouse brain. Neurobiol Dis 54:475-485.
Subbanna et al. (2014) Ethanol induced acetylation of histone at G9A exon1 and G9A-mediated histone H3 dimethylation leads to neurodegeneration in neonatal mice. Neuroscience 258:422-432.
Sun et al. (2012) Morphine epigenomically regulates behavior through alterations in histone H3 lysine 9 dimethylation in the nucleus accumbens. The Journal of neuroscience: the official journal of the Society for Neuroscience 32:17454-17464.
Svinarchuk et al. (1993) Inhibition of HIV proliferation in MT-4 cells by antisense oligonucleotide conjugated to lipophilic groups. Biochimie 75:49-54.
Taniguchi et al. (2017) HDAC5 and Its Target Gene, Npas4, Function in the Nucleus Accumbens to Regulate Cocaine-Conditioned Behaviors. Neuron 96:130-144 e136.
Todtenkopf et al. (2004) Effects of kappa-opioid receptor ligands on intracranial self-stimulation in rats. Psychopharmacology 172:463-470.
Torvik et al. (2019) Explaining the association between anxiety disorders and alcohol use disorder: A twin study. Depress Anxiety. 36(6):522-532.
U.S. Patent Application Publication Nos. 2002/0034765, 2003/0022244, 2003/0153043, 2004/0253645, 2006/0073137, 2018/0256749, 2019/0136199, 2020/0054635, 2020/0113901.
U.S. Pat. Nos. 3,687,808; 4,469,863; 4,476,301; 4,816,567; 4,845,205; 4,946,778; 4,975,369; 4,981,957; 5,001,065; 5,023,243; 5,034,506; 5,075,431; 5,081,235; 5,118,800; 5,130,302; 5,134,066; 5,166,315; 5,169,939; 5,175,273; 5,177,195; 5,185,444; 5,188,897; 5,202,238; 5,204,244; 5,214,134; 5,216,141; 5,225,539; 5,231,026; 5,235,033; 5,264,423; 5,264,562; 5,264,564; 5,276,019; 5,278,302; 5,286,717; 5,292,867; 5,319,080; 5,321,131; 5,354,847; 5,359,044; 5,367,066; 5,393,878; 5,399,676; 5,405,938; 5,405,939; 5,432,272; 5,434,257; 5,436,157; 5,446,137; 5,453,496; 5,455,233; 5,457,187; 5,459,255; 5,466,677; 5,466,786; 5,470,967; 5,472,693; 5,476,925; 5,482,856; 5,484,908; 5,489,677; 5,491,088; 5,500,362; 5,502,167; 5,502,177; 5,514,785; 5,519,126; 5,519,134; 5,525,711; 5,530,101; 5,536,821; 5,539,082; 5,541,307; 5,541,316; 5,550,111; 5,552,540; 5,561,225; 5,563,253; 5,567,811; 5,571,799; 5,571,894; 5,576,427; 5,585,089; 5,587,361; 5,587,458; 5,587,469; 5,591,722; 5,594,121; 5,596,086; 5,596,091; 5,597,909; 5,602,240; 5,608,046; 5,610,289; 5,610,300; 5,614,617; 5,618,704; 5,623,070; 5,625,050; 5,627,053; 5,633,360; 5,639,873; 5,641,870; 5,643,759; 5,646,265; 5,658,873; 5,663,312; 5,670,633; 5,677,437; 5,677,439; 5,681,941; 5,693,761; 5,693,762; 5,700,920; 5,712,120; 5,714,331; 5,714,350; 5,719,262; 5,750,692; 5,766,886; 5,770,196; 5,777,085; 5,821,123; 5,821,337; 5,869,619; 5,877,293; 5,886,152; 5,895,205; 5,929,212; 6,015,886; 6,028,188; 6,054,297; 6,124,445; 6,147,200; 6,160,109; 6,166,197; 6,169,170; 6,172,209; 6,180,370; 6,222,025; 6,235,887; 6,239,265; 6,268,490; 6,277,603; 6,326,199; 6,346,614; 6,380,368; 6,407,213; 6,444,423; 6,528,640; 6,531,590; 6,534,639; 6,548,640; 6,608,035; 6,617,438; 6,632,927; 6,639,055; 6,639,062; 6,670,461; 6,683,167; 6,750,325; 6,794,499; 6,797,492; 6,858,715; 6,867,294; 6,878,805; 6,998,484; 7,015,315; 7,041,816; 7,045,610; 7,053,207; 7,084,125; 7,273,933; 7,321,029; 7,399,845; 7,427,672; 7,495,088; 9,284,272; 9,840,500.
U.S. Reissue Pat. No. RE39464.
Uhari-Vaananen et al. (2018) The kappa-opioid receptor antagonist JDTic decreases ethanol intake in alcohol-preferring AA rats. Psychopharmacology 235:1581-1591.
van der Vaart et al. (2017) The allostatic impact of chronic ethanol on gene expression: A genetic analysis of chronic intermittent ethanol treatment in the BXD cohort. Alcohol 58:93-106.
Veazey et al. (2015) Dose-dependent alcohol-induced alterations in chromatin structure persist beyond the window of exposure and correlate with fetal alcohol syndrome birth defects. Epigenetics Chromatin 8:39.
Vorspan et al. (2015) Anxiety and substance use disorders: co-occurrence and clinical issues. Curr Psychiatry Rep 17:4, doi:10.1007/s11920-014-0544-y.
Wang et al. (2018) Inhibition of the G9A/GLP histone methyltransferase complex modulates anxiety-related behavior in mice. Acta Pharmacol Sin 39:866-874.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for reducing substance consumption by a subject with a substance use disorder (SUD), optionally alcohol use disorder (AUD), the method comprising, consisting essentially of, or consisting of administering to a subject in need thereof a composition comprising, consisting essentially of, or consisting of an effective amount of an inhibitor of a euchromatic histone-lysine N-methyltransferase 2 (EHMT2/G9A) biological activity, wherein the substance consumption is stress-induced consumption, dependence-induced consumption, or both, and further wherein the substance consumption by the subject is reduced as compared to what would have occurred had the subject not been administered the composition and/or had the subject not experienced stress-induced consumption, dependence-induced consumption, or both.

2. The method of claim 1, wherein the substance is alcohol.

3. The method of claim 2, wherein the consumption of alcohol is stress-induced consumption, dependence-induced consumption, or both.

4. The method of claim 2, wherein the consumption of alcohol is associated with a kappa opioid receptor (KOR) biological activity in the subject, optionally wherein the KOR biological activity is associated with stress in the subject.

5. The method of claim 1, wherein the subject is a human.

6. The method of claim 1, wherein the EHMT2/G9A inhibitor is selected from the group comprising (2-(4,4-difluoropiperidin-1-yl)-N-(1-isopropylpiperidin-4-yl)-6-methoxy-7-(3-(pyrrolidin-1-yl)propoxy)quinazolin-4-amine, 2-(hexahydro-4-methyl-1H-1,4-diazepin-1-yl)-6,7-dimethoxy-N-(1-(phenylmethyl)-4-piperidinyl)-4-quinazolinamine (also known as Histone Lysine Methyltransferase Inhibitor (CAS 935693-62-2) or BIX 01294 trihydrochloride hydrate), 6-Methoxy-2-morpholin-4-yl-N-(1-propan-2-ylpiperidin-4-yl)-7-(3-pyrrolidin-1-ylpropoxy)quinazolin-4-amine (also known as UNC1479), 6-Chloro-N-(4-ethoxyphenyl)-2-methylquinolin-4-amine (also known as CSV0C018875), CPUY074020 (CAS No. 902279-44-1), 2-(benzoylamino)-1-(3-phenylpropyl)-1H-benzimidazole-5-carboxylic acid, methyl ester (also known as BRD4770, CAS No. 1374601-40-7), Chaetocin (CAS No. 28097-03-2), A-366 (CAS No. 1527503-11-2), a derivative thereof, a metabolic precursor thereof, a metabolic product thereof, a salt thereof, or any combination thereof; and/or is a nucleic acid that binds to and inhibits the activity of an EHMT2/G9A gene product; and/or is an antibody and/or a paratope-containing fragment thereof that binds to and inhibits the activity of an EHMT2/G9A gene product.

7. The method of claim 1, wherein the administering results in a reduction of dimethylation of lysine 9 of histone H3 (H3K9me2) in the subject, optionally a reduction of dimethylation of lysine 9 of histone H3 (H3K9me2) in the nucleus accumbens (NAc) in the subject.

8. The method of claim 1, wherein the administering is repeated one or more times a day for at least 1, 2, 3, 4, 5, 6, 7, 10, or 15 days.

9. The method of claim 1, wherein the EHMT2/G9A inhibitor is (2-(4,4-difluoropiperidin-1-yl)-N-(1-isopropylpiperidin-4-yl)-6-methoxy-7-(3-(pyrrolidin-1-yl)propoxy)quinazolin-4-amine (also known as UNC0642).

10. The method of claim 1, wherein the EHMT2/G9A inhibitor is 6-Methoxy-2-morpholin-4-yl-N-(1-propan-2-ylpiperidin-4-yl)-7-(3-pyrrolidin-1-ylpropoxy)quinazolin-4-amine (also known as UNC1479).

11. The method of claim 1, wherein the subject has a stress-related and/or anxiety-related disorder and/or a disorder exacerbated by stress and/or anxiety, optionally wherein the stress-related and/or anxiety-related disorder and/or a disorder exacerbated by stress and/or anxiety is selected from the group consisting of post-traumatic stress disorder (PTSD), panic disorder, social anxiety disorder, general anxiety disorder, and major depressive disorder.

12. The method of claim 1, further comprising administering at least one additional therapy to the subject, optionally wherein the at least one additional therapy comprises, consists essentially of, or consists of a behavioral therapy, optionally a cognitive behavioral therapy.

13. A method for reducing relapse vulnerability in a subject that has a substance use disorder (SUD), optionally Alcohol Use Disorder (AUD), the method comprising, consisting essentially of, or consisting of administering to a subject a composition comprising, consisting essentially of, or consisting of an effective amount of an inhibitor of a euchromatic histone-lysine N-methyltransferase 2 (EHMT2/G9A) biological activity, wherein the substance use disorder is associated with stress-induced consumption, dependence-induced consumption, or both in the subject, and further wherein the effective amount is sufficient to reduce the incidence of stress-related alcohol consumption, dependence-related alcohol consumption, and/or another substance consumption by the subject as compared to what would have occurred had the subject not been administered the composition and/or had the subject not experienced stress-induced consumption and/or dependence-induced consumption.

14. The method of claim 13, wherein the subject has stress-related alcohol consumption, dependence-related alcohol consumption, or both.

15. The method of claim 14, wherein the stress-related alcohol consumption, dependence-related alcohol consumption, or both is associated with a kappa opioid receptor (KOR) biological activity in the subject, optionally wherein the KOR biological activity is associated with stress in the subject.

16. The method of claim 13, wherein the subject is a human.

17. The method of claim 13, wherein the EHMT2/G9A inhibitor is selected from the group comprising (2-(4,4-difluoropiperidin-1-yl)-N-(1-isopropylpiperidin-4-yl)-6-methoxy-7-(3-(pyrrolidin-1-yl)propoxy)quinazolin-4-amine, 2-(Hexahydro-4-methyl-1H-1,4-diazepin-1-yl)-6,7-dimethoxy-N-(1-(phenylmethyl)-4-piperidinyl)-4-quinazolinamine (also known as Histone Lysine Methyltransferase Inhibitor (CAS 935693-62-2) or BIX 01294 trihydrochloride hydrate), 6-Methoxy-2-morpholin-4-yl-N-(1-propan-2-ylpiperidin-4-yl)-7-(3-pyrrolidin-1-ylpropoxy)quinazolin-4-amine (also known as UNC1479), 6-Chloro-N-(4-ethoxyphenyl)-2-methylquinolin-4-amine (also known as CSV0C018875), CPUY074020 (CAS No. 902279-44-1), 2-(benzoylamino)-1-(3-phenylpropyl)-1H-benzimidazole-5-carboxylic acid, methyl ester (also known as BRD4770, CAS No. 1374601-40-7), Chaetocin (CAS No. 28097-03-2), A-366 (CAS No. 1527503-11-2), a derivative thereof, a metabolic precursor thereof, a metabolic product thereof, a salt thereof, or any combination thereof; and/or is a nucleic acid that binds to and inhibits the activity of an EHMT2/G9A gene product; and/or is an antibody and/or a paratope-containing fragment thereof that binds to and inhibits the activity of an EHMT2/G9A gene product.

18. The method of claim 13, wherein the administering results in a reduction of dimethylation of lysine 9 of histone H3 (H3K9me2) in the subject, optionally a reduction of dimethylation of lysine 9 of histone H3 (H3K9me2) in the nucleus accumbens (NAc) in the subject.

19. The method of claim 13, wherein the administering is repeated one or more times a day for at least 1, 2, 3, 4, 5, 6, 7, 10, or 15 days.

20. The method of claim 13, wherein the EHMT2/G9A inhibitor is (2-(4,4-difluoropiperidin-1-yl)-N-(1-isopropylpiperidin-4-yl)-6-methoxy-7-(3-(pyrrolidin-1-yl)propoxy)quinazolin-4-amine (also known as UNC0642).

21. The method of claim 13, wherein the EHMT2/G9A inhibitor is 6-Methoxy-2-morpholin-4-yl-N-(1-propan-2-ylpiperidin-4-yl)-7-(3-pyrrolidin-1-ylpropoxy)quinazolin-4-amine (also known as UNC1479).

22. The method of claim 13, further comprising administering at least one additional therapy to the subject, optionally wherein the at least one additional therapy comprises, consists essentially of, or consists of a behavioral therapy, optionally a cognitive behavioral therapy.

23. The method of claim 13, wherein the subject has a stress-related and/or anxiety-related disorder and/or a disorder exacerbated by stress and/or anxiety, optionally wherein the stress-related and/or anxiety-related disorder and/or a disorder exacerbated by stress and/or anxiety is selected from the group consisting of post-traumatic stress disorder (PTSD), panic disorder, social anxiety disorder, general anxiety disorder, and major depressive disorder.


SEQUENCE LISTING (32 sequences)

SEQ ID NO: 1 - DNA, 20 bp, Mus musculus
  source 1..20: mol_type = other DNA
tgcctatgtggtcagctcag

SEQ ID NO: 2 - DNA, 20 bp, Mus musculus
  source 1..20: mol_type = other DNA
ggttcttgcagcttctccag

SEQ ID NO: 3 - DNA, 21 bp, Mus musculus
  source 1..21: mol_type = other DNA
aggtcggtgtgaacggatttg

SEQ ID NO: 4 - DNA, 23 bp, Mus musculus
  source 1..23: mol_type = other DNA
tgtagaccatgtagttgaggtca

SEQ ID NO: 5 - DNA, 21 bp, synthetic construct
  misc_feature 1..21: note = Artificially synthesized short hairpin sequence
  source 1..21: mol_type = other DNA
gagccacctccaggtggttgt

SEQ ID NO: 6 - DNA, 21 bp, synthetic construct
  misc_feature 1..21: note = Artificially synthesized short hairpin sequence
  source 1..21: mol_type = other DNA
aaatgtactgcgcgtggagac

SEQ ID NO: 7 - DNA, 4045 bp, Homo sapiens
  source 1..4045: mol_type = other DNA
agagatgcggggtctaccgagagggagggggttgatgcgggcccgggggaggggtcgtgcggcccctccgggcagccgaggccgcggaaggggggggccccacagaggaagaggtaggccccggagcctactctctcttcccagggcccaggcatcctggaccccccaactctctactgggctgaccagccctcctgtcccttgtctcccctcccagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatgagaacggaccacaccctctctccccagcatggatggccacagctcagccgcctcctctgccaccagctgctcgcagcccatgcctgggggtgctgccatcttctctccccaccaccctttcacacattcctgaccagagatcccagccaggccctggaggtctgacagcccctccctcccagagctggttcctccctgggagggcaacttcagggctggccaccccccgtgttccccatcctcagttgaagtttgatgaattgaagtcgggcctctatgccaactggttccttttgttctcaataaatgttgggtttggtaataaaaaaaaaaaaaaaaa

SEQ ID NO: 8 - protein, 1233 aa, Homo sapiens
MRGLPRGRGLMRARGRGRAAPPGSRGRGRGGPHRGRGRPRSLLSLPRAQASWTPQLSTGLTSPPVPCLPSQGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 9 - DNA, 17940 bp, Homo sapiens
  source 1..17940: mol_type = genomic DNA
cggcccccagcgcaagcggcgatggcggcggcggcgggagctgcagcggcggcggccgccgaggtgcggaaggggagggggagaggcgggtgcatgcccgcgcgcgcgcccgggggaagcgcgcgcccgtgcaatgctccgggggtgcaacggggccggggggcccgggcgggacttgcagcaatccagggggccagcaccgggggcggatgaggggaccagggtgtgggaggtggggggagtgggcacggaggcgcgcgtgcagccgccggctgcccctgagtgggcgacgggcgccggggccccaggagcacgcgtgaggggcacgaggggccgtccaccgggctggggggcagcgggtgctggaggggttggtgcccggggtccccgcggcctgggggacaaagggggagcggcgcgtgcagccgggaggagggggcggggccggggcgcggaggccccgccccctccccctcctcttctctcggccccagagatgcggggtctaccgagagggagggggttgatgcgggcccgggggaggggtcgtgcggcccctccgggcagccgaggccgcggaaggggggggccccacagaggaagaggtaggccccggagcctactctctcttcccagggcccaggcatcctggaccccccaactctctactgggctgaccagccctcctgtcccttgtctcccctcccagggggaggcccccgctgagatgggggcgctgctgctggagaaggaaaccagaggagccaccgagagaggtgagtgcagctaaaacgggccccaattggaccgtcaccttccctaaacacaggatctcctgagcccttagctctgaactttttctcttcctctacagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggtgagaggctgggtagcctggggggttggtgggaaatttctgactcttgccttctttcttatcttgccttttgcttgcctgttttctaggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacaggtgagtgggatgggagagatgggataatcaaccataaacctttggtgagctcttttacagtttcacttaaggcttctactccatttactcattcacatatttaagcaaggggctgctgtgtgtcaaatattaggctgtgcttgatctgtgaaccaacagacaggttcctctcttcctgcagtttacattcttgggtggtgaaagtttgatgttaaagagtcaattatatagttaatgtcttaattactcttatgataatgacaaggtaagggaatttcaaaccactcttcctttctacctcaaagcaagcccttggggaactcatctgtcactgctctatttgtaaagtttttcctgggaatcccagtgggaaaggagatgagggccacagaaataatagaggagggaagtgacaagggcaccagaagctttatgaccaagttctagggttcttgggtcatacctattgttgacctaaaggaaaaaactgaggcaaacttaacattaagtaaagtatatttggtcaggttttgaggacagcctttaggagcaaagattcaagttgccgtgaaaatacactctgattagcagcagttataagtaggggtttttttgtttgttttgttttgtttttgaagacagtttcactatgttgcccaggctggtctcaaactgctgggcttcagcaatcttcctgccttgacctcgcaaagtgctgggattacaggtgcaagccaccacaaccagcccacaagtaggtttttaaaggaaaagaagattggatgacgtgaagatattgaaaaaaaacaggctgactctgggcacactgcgtaggagttagccctgctctggaaggagccgcaaaaagaaaaaaaaaaagataaaaattttttaaaagattgaagaaataacaaaggaaaagaagaggcagttcctaaattgtttaccagtttacattagaataacaagctattgatctgctatacattgttctttgtctcacagatattaggcacatgaagataatgggtgaagcagcaccctagtcaggtacaaaatgcctttaagcagttgcctccaggcatgggccctaaccgttccatgctgacttctcgctgagcctgatcaattttgcctgcttcacatagcacaggttgctatgagctacctttcctttctcactatgataaacattaatttttatagttgcaattcattttgtaaagaatagggaagaaaaggaaaattgaatttataaattaggtacagtgagagattaacaacaataataacataatagcgtaatcctaacagtaggccaggcgtggcttgcctataatctcaacactttcagaggccaaggccggcaatcacttgaggtcaggagttcgagaccagcctggtaaacatgtccaaaccccgtctctaactaaaataataataataataataaattagccaggtgtggtggcgtgcgcctgtaatcccagctagtcgagaggctgaggcaggagaatcacttgcagtgagccaagatcgcgccactgcggttcagcctgggcaacagagcgagactcaaaaaaaatagcacaattctaacaatatgccaccatcactgctgatgaactggataagaggggactagtgtcacgtaaaatttaccatctaaaaaacaattttaggccaggcgcagtggctcacgcctgtaatctcagcactttggaggccgaggcgggaggatcacctgaggtcaggagtttgagaccagcctggccaacatagtgaaaccctgtctctactaaaaatacaaaaattaggccaggcggggtgcctcacgcatgtaatcccagcactttgggaggccaaggcaggtggatcacctgaggtcaggagttcgagaccagccggacgaacatggtgaaaccccatctctattaaaaatacaaaattagccgggcatggtggtacatgcctgtaacagtccgctacttgggaggctgaggcaggagaatcgcttgaatccaggaggcagaggttgtagttagccgagatcctgccattgcactccagcctggacaacaagagcaaaactctgtctcaaaaaaaaaaacaaaacaaaaattagccgggcgtggtggcatgtgcctgtagttccagctactcaggaggcagaggtgagagaaacgcttgaacctgggaggtagaggctgcagtgaactgagatcgcgccactgcactccagcttgggcaacagagtgagactccatctaaaaatttaaaaataaaataaaaaacatttttaagtatataatttaatggtattaaatactttcatcatgtgcaatcatcagtcaccataactcatcttgtaaaactgaaactctgtacccattaaaacataactctccattccctccaccccctagcccctggcaaggaccactgtcctttctttctctataaatttgagtaagtactttatctaagtgtaatcatacagtatttatccttttgtgactggcttatttcacttagcataatgtcctcaaggttcatctatgttgtaccatgtcagaatttctttcctttcacctgggcagcataccaagaccccatctctaaaagaagtttaaaagaatgtttcaaaggccaggcccagtgactcacgcctgtaatcccgtactttctggggaggatcacttgacaccaggagttcaagaccagcctgggcaacatggcaagacctcttctctaccaaaaaaaaaaattaagaagacattagttaggcattgtgacatgtgcctgtaatcccagcttcccaggaagctgaggcaggagaatggcttgagtccagttcgaggctgaagtgagccatagtcatgccactgcactccatcctgggtaacagaatgagaccttatctcttaaaaaaagtatatctttcctttttatggctgaatgttattccattgtgtataaatgccacattttgcttatccattcatctgtcagtggcacttgagttgcttccgcacctcagctgttgtaaacagtcctgctgtgtatatggccacatataaaaggtcttttaactttattaagctttgttttctcatctgtaaaatggaaaagggcctgctctgcatggttgcagagatgagtgagtgttagtgtgcttgacactcttcctctgaggaaatagcacccgtggttgccagtttgtggctactggagcctcacatggcatttggaggtgtggctcactaccccacccccgcctggggctccaggctaacccaggcaaaactgtaggtgctctctgaccctcagtgtgctccctctcggcccacagcccccggtccctgagaagcggccccctgaaatacagcatttccgcatgagtgatgatgtccactcactgggaaaggtgacctcaggtcagtcccatcccccacccccggtggcttgatgtggtctgttctgaaggtttcggtgataggccccacaccattctctctcttcttccagatctggccaaaaggaggaagctgaactcaggaggtggcctggtaagcagcagaggcagagcctctacccctcgggggctgctctgagggagggtcagaggtggggctgcatcccatcctcagtagaaatgcgcagggtgtcagtggcctcaatttttctgatctttttctagtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaaggtgagacagagctggtctgccctccccgccggtgctcccccagggctggggctcactgtccagtcctccttgttccctgtttttctgctttctgtcctttttatcttctgaatatccactaagtcattgccccatgctgtctataactccttttttttgtggggggagacggaatctcattctgtcgcccaggctggagcgcaatatcacgatctgggctcactgcaacctctgtctccagggttaaagcaattctcctgcctcagcctcccgagtagctgggattacaggcacctgccaccacgcccggctaatttttgtatttttagtagagacggggtttccctgtgttggccaggctggtctctaactcctgaccttagttgatccacccaccttggcctcccaaagtgctgggattacagctgtgagccaccgcgcccagctgtaactcattttcaatgtgtattctgcccgtttcatccacaaaggtggccaccaccctgcacatgggatttagtattcttttttttttaatcagctaatgaaatgaccatttaacagacatgtactgtttaaaaaaagaaaacaaaaaaagaaatgaccagaaagcacttttttaaaacatacgttatgctgtttatataaaattcaaaagcattgcattgtcatttatttgcttatttcgttgctctttgtctggttttatcttatatgtttgcctacacagtagaatgtttagtctctctgattttgccttttatttaacattttatcatgaaaacttgcaagcataatcaaaagagtagaatagggagcccccatgtacccaatacacagctccagccattatcagtacacagtccatcttgacccatctgtacatctcccagcacacacacctactaggttattttaaagcaaatcccagacctcatatcattttacctaaacatatatccatatagatttgtaaatataaggcctctttttgtcgttcaacatcattacactactgttttcacacctaaaaaaacttaccgatttcttaataccatcaaatatcctgtcagtcttcacatttcctcagctctcacaaatggtttttgtcaagatccaaatgaggcccacaccttgatttgattaacatttgtcttaggtcagccaggcacagtggctcacgcctgtaatcccaatactttgggaggccaaggcgggcagatcacgtgaggttgggagttcgagaccagcctgatcaacatggagaaaccccgtctctactgaaaaaaatacaaaattagctggacgtagtggtacgtgcctgtaattccagctactcgggaggctgaggcaggagaatcgcttgaacctgggaggtggaggttgcagtgagccaagatcgtaccattgcactccagcctcggcaacaagagcgaaactccgtctcaaaaaaaaaaaaatttttttttgtcttaggtctctctcagtatttaggtttccccatcttctttgtgtttgtttgtttgtttgtttttgttttttgagatggagtctcacactgtcacccaggctggagtgcagtggcgtgatcttggctcactgcaagctccacctcctgagttcaagcaattctcctgcctcagtctcccaagtagctgggattacaggcacccaccaccatgcccagctaattttttgtatttttagtagaggcagggtttcactatgttggccaggctggtctcaaactcctgatctcgtgatctgcccacctcggcctcccaaagcgctgggattacaggcgtgagccaccacacctggcctgatttcctcatcttctaaaaaaaaattttttggcccaggcgtggtggctcacgcctgtaatcccagcactttgggaggcaggtggatcacctgaggtcaggagtttgagaccagcctagccaacatggtgaaaacacatctctaataaaaatacgaaaattagctgggtgtggtggcgggtgcctataatcccagctactcaggaggctgaggcaagagaattgcttgaatccaggaggtggaggttgcagtgagctgagatcacaccattgcaatgcagcctgggcaacaagagcaaaactcaataaataaataagaaagattttttttttttgccatttatttgtcaaagaaaccagctcagttgtcctacagaatttttcacatttaggagttggctgattgggtggagattgacacatcctctctgcccatatttcttgtaaactgatagatctagaggcggaatcagcttcaggtgccaagaagacttgacagtgggtgcaagtgcttctggccacatcacatcaggatgcatggaacatctaatctggtcatttttcttgttagtgataagattgaccagtgggttcaggtgatgtcagcctgaacctttcatataaagtttctcatgagctttgtgtctagtgtttttagcaaccattggtggtcttgcctgtttatgaacatcataaaaggggtatcaaagtatgtttatcttctggtacttgctttttgttttttaacttaatatcacattaccaagatctgtgcacattgctgtctgtaactttttcactgctgtgtaatattccatgcaccctctctttatgtggccctttgtctcctctgctacccacacttatccccagcagccatccgtgggtagtggcggggggcaatggtggtggtggttttctttactcacctcattgtctgagtcctgggggttggatatgtctgttctgcctctctcccagtctgaagttgaagctctaactgaacaactaagtgaagaggaggaggaggaagaggaggaagaagaagaagaggaagaggaggaggaagaggaagaagaagaggaagatgaggagtcagggaatcagtcagatagggtaagagacggaggctgatatctccagaggagtgggagactatggggctggaggtctggtcctgaaggtgttgggggggcccctgggggtgagggttcctaactcctcctcctcccccttcccagagtggttccagtggccggcgcaaggccaagaagaaatggcgaaaagacagcccatgggtgaagccgtctcggaaacggcgcaagcgggagcctccgcgggccaaggagccacgaggtgaggaggctctgctgcttttgggtgccctccagcccccgcccggccccccagagtgtgtgcacgcacacacacgctctcgcatgtccacctgcatgtacccacgcgtccaggcacctgtgagctcgcactctcactctctctgtctctgtgtcaggagtgaatggtgtgggctcctcaggccccagtgagtacatggaggtccctctggggtccctggagctgcccagcgaggggaccctctcccccaaccacgctggtaattgccaattgccgggacagggagccactagggggcgacctcagggcaggagggaaagggaaggaggggaaccacgccagagccggggtgtccatggccaggctttaggggttctggggcatggcggggtggggtagggagggagtgagaggacccgccaggggtcccagtaggtgaggtgcagagcctccctcagctcctcttttcctccatccaaggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagaggtggggccgtgggctggtgggagaggtgccagggcgtccagtccccggccccagcctcacgctctcttctcacccatcctcactgcgcacagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcggtgagtgaccagtggggcagacaggtagcatgccctgtggcagagggggccccagtaacctgaccatgtcgtttccctgctcccagggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccaggtactggcctcccccttctgtactgtctgttccctcccccacccctattgctcctggacatgagctccttcttccacagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggtgagtggggagttgctcaggcacagcaactggggctgaggccagaggagtggtgtcgaggctgatgctggaatctgaggagctccccttctctccccgctcccgtgctcccttggcaggcggaagaagctccgtttccaccctcggcagttgtacctgtccgtgaagcagggcgagctgcagaaggtgatcctgatgctgtgtgagtgccacccattccttcagcagaccttgaccaagcttcatgtatataccaggcactgggtacagggccaggagtacagtcgtgaaggacacagtccttgccctgaaggacttagtgtggtggggaaaagacacatgtaacccataacgatggggatgtggtgactcataggtgctgggtcctgttctgagtgcttatacctgttagccttggcttgcacagagaggttagggaccttgcacagtgcacagtggtactgagaagcaaggcagggtttgaattcaggcagagctgatgcctttgactccttattctgataaatgctgtggtggaggcaagctcggagaatgccaggaggggccctgatccagcttggggccctgagaagccttcctgaaagaagtgccacctgccccctagcttgcttaccacttgtccctccctctcccggtgtggcgggctctccccgcagtggacaacctggaccccaacttccagagcgaccagcagagcaagcgcacgcccctgcatgcagccgcccagaagggctccgtggagatctgccatgtgctgctgcaggtcagcacgggcccggccccatgcctcattcaccaggcccttaggcccctcccctgccccatgcctccctggtgccagccctcctgccccctcacaggctggagccaacataaatgcagtggacaaacagcagcggacgccactgatggaggccgtggtgaacaaccacctggaggtagcccgttacatggtgcagcgtggtggctgtgtctatagcaaggtgtgcgcgcaggcagcagggcgtggcccccggagtcagggaccaggtttggggtgccagcccaaaggctcatttgcccgtgtctccctcaggaggaggacggttccacctgcctccaccacgcagccaaaatcgggaacttggagatggtcagcctgctgctgagcacaggacaggtggacgtcaacgcccaggtcagcggcccacccagcccagcccctctgggtccctggtgcctgggttccttggcttggcctcaggctttgggccgcctttcctacatctgacctcacactgctcgtgtctgtcctcattgctttcttcttcctcatttgtatttctcttcattcttttctttctcgtttctttctctttatcaagtttcagaataatcagttcctgtcttaatctctgaaagtgtccagtgaggctttttattaactcttgggttgtcatacattggatgtgttttagtcatttattggcattcttctcagtctttgaattattccatcattgtccagaagaagcccctttaagaggcctctgtggctgtgacatgacccagtggcccagtggcctttgagagcctcctgacctttcagcacatttcctgcccccaactcagaatccaccatttctctaagaaactctgcttcctttatgggaaacttttttttttttttttgagacggagtctcactctgttgcccaggctggagtgcaatggcgtgatcacagcttactgcaagctccgcctcccaggttcacgccattctcctgcctcagcctcccgagtagctgggattacaggcgcccaccaccatgcccggctaatttttgtatttttagtagagacggggtttcaccatattggccaggctgatcttgaactcctgacctcgtgagccacagcgcccggccagaagcattttttagagaatacaatctatgttcctaggagtgtttattgttattgggttgtcgctgcttctaggacttttgagtgggcagacttaggaaatttttgttcgttttttcttttaagagacagggtcttgctatgtcacccaggctgaagtacagtagcagttcacagacagtcatagctctctgcagcctcgaaatcctgggctcaagcaaccctcccatctcagtcacatgagtagctgggactacaggcatgcacctccatgaccagctcctggctgtattttttggaaagagaacaattactttattttctctctggcatcagatggtagacgctactagtcccatttttgtttgtttgttttttgagacggagtcttgctctgtcgcccaggctggagtgcagtggcacgatcttggctcactgcaagctccgcctcccgggttcacgccattctgcctcagcctccctagtagctgggactacaggcgcctgccaccacgcccagctaattttttctattttttagtagagacagggtttcaccgtgttggccacaatggcctccatctcctgacctcgtgatccgcccgcctcggcctcccaaagtgctgggattacaggcatgagccaccgcgcccgaccctactagtcccattttttagatgaagaaactgcgacccagagacgttaagtgacttacccagggtcccacagcttgtgaatggtggagctgggattcacacacacagccaatcttgaccatttgctgctttggtgagcagggccagctgcccccaccctcactcccttcccacccacaggacagtggggggtggacgcccatcatctgggctgcagagcacaagcacatcgaggtgatccgcatgctactgacgcggggcgccgacgtcaccctcactgacaacgtgagtgagcgtttggttgaggtagggcagccccaggcccctgagcaaggtggaggctggattcaagggcccagctgcctgcacctcatctgttcccctcctacctccacaggaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgtgagcccctgccccgcccttgatgcccctgcccccacttcttgcagcctcagaccctgcattgggcatcctgtcccctcttcaggttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccggtgagccctgggcccctctctgcacccaagaccacctcctccccacccataccctccccaggctccattgcatcctcacaacattccccaacccctccccatgcaccactgaatccccagaaccacccacaggccccctccccactagggttgccagatacagcaaataaaaatagaagacacccagttaaactggaatttcagatgcacagatgcatacttttttagtgtgagaatgtcctgtgtaatttttgggacatactaaaaagctatttcttgtttatctaaaattctatgttaactcgctgtcctgcatcttacctggcaaccttactcccctgcccccagccccaggaacctccccaactctcttccttatacctggagtagctcctcacccccttttccagcttcccagccccttcttcccgtcttctgcagggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcaggtgagtagggggcccggggcctccccaaaaggacaccgagagctgccaagtacagttggacaagcttcaaaccagcaaaggcacctggccaaggaagggttggctgaaagggagccctggtgtggggtcgcatccccaccaggggagggatccttttcctaggtcacagcaaggtatcatatgagctcccagtagccctggggacaccaccatactgattacccgtcaccctaggaccatcaccacccactgttgtaacacatggaccccaaagaaaggacagtggttagggggtcagggggcatcaagagtagaggggcccagaccgatcatggggttgagctgacctctgcctcctcgccccagcactgcacgtgtgtggacgactgctctagctccaactgcctgtgcggccagctcagcatccggtgctggtatgacaaggtgcgtgccccttgccttggccacagacacccggcccctccttcagaggagccagcctccaataaaccagctccctgggaccatgtccctcacctcctaccctgtgggggaagtgactgagaggactgggcaaggcctgggaatcctgggatgtggctgggacagcgatgggcgtgtgcccaggtctatgcaaaccagggatgatgctcctgcatttcgtgctctcggatctcagagcctgtgtgacctccccaaccccaacttccctgtctctgtagagtcttggggttcctgggtgggcagctgaggtgggccccacagagaaaccactatcctcccctccgcaccctaggatgggcgattgctccaggaatttaacaagattgagcctccgctgattttcgagtgtaaccaggcgtgctcatgctggagaaactgcaagaaccgggtcgtacagagtggcatcaagtgaggccctgctccacagcccaccctctccccacctcaccctttcctccctgcccctggggcttttccagggcctccacccaacagcgccatctctcctacccccagggtgcggctacagctctaccgaacagccaagatgggctggggggtccgcgccctgcagaccatcccacaggggaccttcatctgcgagtaagtcaccgggagcaccttaacacctggtgggaaggggatacatggccactgctgactgccaggcctccctctcagccatctgccctgatgagtcacatgtctccatctggccactctccagccagcctcctctgacccctattcctgcagctgagcagccagccgcctctgacccccatccccccagctgagaatggcacctgcctccactctgcccagaccacagagcttgtcttccctcccctccacctccaccctctaccttcccctccaccccctcctcgcccttcctagctggagaaagtgccagtttgggagacaaacagacttgggtttgagcccctctctgcttctggatgttcttggccaagttacttaacctctttgagccttatttttcttgtctgtagagtggaggtgataacagctacctatctgtccacctcctcaggggcctgtccctctccatcagttctttctctgcccagcatctgtaatctccccctttctcttggctgccatcccacaaccctcaatcttactttagtctcctttggctcttttttttttttttttttaatttattttttgagacggagtttcactcttgttgcccaagctggagtgcaatggcacaatcttggctcactgcagtctccacctcccaggttcaagcgattctcctgcctcagcctcccaagtagctgggattacaggtgcgcaccaccacacccggctaattttttgtatttttagtagaaacggagtttcaccatgttagccaggctggtctccaactcctgacctcagatgatccgcccacctcggcctcccaaagtgctgggattacaggcgtgagccaccgtgcctgaccattctttggcctttaaaaaagatagtcctctaaccatacttgcctgttcaacagaccatccagtttctttccatcatgtcatctctcaacttctctggcaagcagccccttcctgctgggcctcagtttccccaccatccactcactgcctgtccctgccaaaatcggcctttgcccttcccactctgctgaagctgtctcttgacagtcacttcccacccacgtcctctccgtgtgtctgaatctcccatgtctgctcaccctccttagatagctggctcctctcctccctctggtgtccagatatgtgtgggtgctcttgccccgaatcatgccttgagttccttgccactcctgtacctctggctcttcctgcagtcctggcccctctcctgttccatatcgcccattcctgccgggtggctcctggaacacaaacctctctgtgtccaaacccgaacttccccacccaggccaactgcccctacacattaatccccttaaccacagagcttgttgtctgtctctgtgctgactgaaggcttcctctgtcaaggctggaatgctgccctttcacttgcccaccactgctgctccaggccttcccttccccacacctgccagccctgcccattccagattccacaggcttgtgaagagagatggggcctggagccacctcctaatagcccacactcaccttcagaaccatggattcctgtcccacaggtatgtcggggagctgatctctgatgctgaggctgatgtgagagaggatgattcttacctcttcgacttagacaacaaggtgagcaggagaccctccttaccctgctgctccccagggctggctttcaggagcctctgggaagtcagcaaatggaaacctggggaggagggactggggagtcagtgggtggggagggcaagcagggtcgggggagatgtgaagagtgccatccccctgccccaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtgagactctaggggtctctaggggctctgccagggggtgggagatgcagcaaacctgggacctgaaggctggcatgtacctggtgtccaggcatggagtagtgcagagcttctcagatttcagtgtacataagtatttcctggaggacttggggaaacagtcctgtgcctcccagagagattttgaatcagtaggcttgggatggggcccaggattctgcatttctaaaaagtccccaggcaatgctgatgctgaaagtccatagaacatacaaggggctcagatcccatctggagaagataggttgggtgtcgggtccctctcagggagcaggatccacagggtcttcggggtgggctcttggtggctgggtccaggtcccacaagctcctgttttcctctgacaggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatgagaacggaccacaccctctctccccagcatggatggccacagctcagccgcctcctctgccaccagctgctcgcagcccatgcctgggggtgctgccatcttctctccccaccaccctttcacacattcctgaccagagatcccagccaggccctggaggtctgacagcccctccctcccagagctggttcctccctgggagggcaacttcagggctggccaccccccgtgttccccatcctcagttgaagtttgatgaattgaagtcgggcctctatgccaactggttccttttgttctcaataaatgttgggtttggtaataaa

SEQ ID NO: 10 - DNA, 3979 bp, Homo sp.
  source 1..3979: mol_type = other DNA
cggcccccagcgcaagcggcgatggcggcggcggcgggagctgcagcggcggcggccgccgagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggagtgaatggtgtgggctcctcaggccccagtgagtacatggaggtccctctggggtccctggagctgcccagcgaggggaccctctcccccaaccacgctggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatgagaacggaccacaccctctctccccagcatggatggccacagctcagccgcctcctctgccaccagctgctcgcagcccatgcctgggggtgctgccatcttctctccccaccaccctttcacacattcctgaccagagatcccagccaggccctggaggtctgacagcccctccctcccagagctggttcctccctgggagggcaacttcagggctggccaccccccgtgttccccatcctcagttgaagtttgatgaattgaagtcgggcctctatgccaactggttccttttgttctcaataaatgttgggtttggtaataaa

SEQ ID NO: 11 - protein, 1210 aa, Homo sapiens
MAAAAGAAAAAAAEGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 12 - DNA, 3877 bp, Homo sapiens
  source 1..3877: mol_type = other DNA
cggcccccagcgcaagcggcgatggcggcggcggcgggagctgcagcggcggcggccgccgagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatgagaacggaccacaccctctctccccagcatggatggccacagctcagccgcctcctctgccaccagctgctcgcagcccatgcctgggggtgctgccatcttctctccccaccaccctttcacacattcctgaccagagatcccagccaggccctggaggtctgacagcccctccctcccagagctggttcctccctgggagggcaacttcagggctggccaccccccgtgttccccatcctcagttgaagtttgatgaattgaagtcgggcctctatgccaactggttccttttgttctcaataaatgttgggtttggtaataaa

SEQ ID NO: 13 - protein, 1176 aa, Homo sapiens
MAAAAGAAAAAAAEGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 14 - DNA, 4252 bp, Homo sapiens
  source 1..4252: mol_type = other DNA
ggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcataccaagaccccatctctaaaagaagtttaaaagaatgtttcaaaggccaggcccagtgactcacgcctgtaatcccgtactttctggggaggatcacttgacaccaggagttcaagaccagcctgggcaacatggcaagacctcttctctaccaaaaaaaaaaattaagaagacattagttaggcattgtgacatgtgcctgtaatcccagcttcccaggaagctgaggcaggagaatggcttgagtccagttcgaggctgaagtgagccatagtcatgccactgcactccatcctggcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggagtgaatggtgtgggctcctcaggccccagtgagtacatggaggtccctctggggtccctggagctgcccagcgaggggaccctctcccccaaccacgctggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgtgagtgagcgtttggttgaggaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatgagaacggaccacaccctctctccccagcatggatggccacagctcagccgcctcctctgccaccagctgctcgcagcccatgcctgggggtgctgccatcttctctccccaccaccctttcacacattcctgaccagagatcccagccaggccctggaggtctgacagcccctccctcccagagctggttcctccctgggagggcaacttcagggctggccaccccccgtgttccccatcctcagttgaagtttgatgaattgaagtcgggcctctatgccaactggttccttttgttctcaataaatgttgggtttggtaataaaaaaaaaaaaaaaaa

SEQ ID NO: 15 - protein, 1008 aa, Homo sapiens
MSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNVSERLVEEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 16 - DNA, 4133 bp, Homo sapiens
  source 1..4133: mol_type = other DNA
agagatgcggggtctaccgagagggagggggttgatgcgggcccgggggaggggtcgtgcggcccctccgggcagccgaggccgcggaaggggggggccccacagaggaagaggtaggccccggagcctactctctcttcccagggcccaggcatcctggaccccccaactctctactgggctgaccagccctcctgtcccttgtctcccctcccagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggagtgaatggtgtgggctcctcaggccccagtgagtacatggaggtccctctggggtccctggagctgcccagcgaggggaccctctcccccaaccacgctggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatgagaacggaccacaccctctctccccagcatggatggccacagctcagccgcctcctctgccaccagctgctcgcagcccatgcctgggggtgctgccatcttctctccccaccaccctttcacacattcctgaccagagatcccagccaggccctggaggtctgacagcccctccctcccagagctggttcctccctgggagggcaacttcagggctggccaccccccgtgttccccatcctcagttgaagtttgatgaattgaagtcgggcctctatgccaactggttccttttgttctcaataaatgttgggtttggtaataaa

SEQ ID NO: 17 - protein, 1267 aa, Homo sapiens
MRGLPRGRGLMRARGRGRAAPPGSRGRGRGGPHRGRGRPRSLLSLPRAQASWTPQLSTGLTSPPVPCLPSQGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 18 - protein, 1274 aa, Homo sapiens
MRGLPRGRGLMRARGRGRAAPPGSRGRGRGGPHRGRGRPRSLLSLPRAQASWTPQLSTGLTSPPVPCLPSQGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNVSERLVEEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 19 - protein, 1240 aa, Homo sapiens
MRGLPRGRGLMRARGRGRAAPPGSRGRGRGGPHRGRGRPRSLLSLPRAQASWTPQLSTGLTSPPVPCLPSQGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNVSERLVEEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 20 - protein, 1217 aa, Homo sapiens
MAAAAGAAAAAAAEGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNVSERLVEEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 21 - protein, 1183 aa, Homo sapiens
MAAAAGAAAAAAAEGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNVSERLVEEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 22 - protein, 1233 aa, Homo sapiens; identical to SEQ ID NO: 8

SEQ ID NO: 23 - DNA, 3980 bp, Bos taurus
  source 1..3980: mol_type = other DNA
atggcggcggcggcgggagctgcagcggcggcggccgccgagggggaggcccccgctgagatgggggcgctggtgctggagaaggagcccagaggagccaccgagagagttcatggctctttgggggacacctctcgtagtgaggagaccctgcccaaggccaaccccgattccctggagactgctggcccctcatccccagcctctgtcacagtcactgtcggcgatgagggggctgacacccctgtaggggccacaccactcattggggatgaacccgagaatctcgagggagatggggacctccatgggggtcgcatcctgctgggccatgccacaaagtcgttcccgtcttcccccagcaaggggggtgcctgtcccagccgagccaagatgtcaatgacaggggccggaaaatcacccccatcagttcagagtttggctatgaggctgctaagtatgccgggcgcccaaggggcgccagcagcagggcctgaaccccctccggccacagccagcccagaggggcagcccaaggttcatcgagccaggaaaaccatgtccaaaccaggaaatggacagcccccagtccctgagaagcggccccctgaagtgcagcatttccgcatgagtgatgacgtgcactcgctggggaaggtgacctcagatgtggccaaaaggaggaagctgaactcaggaggtggcttgtcagaagagttggcttctacgcggagttcgggagaagtgaccctggagaagggggaccccgggtccctggaggagtgggaaacggtggtaggggatgacttcagcctctactatgattcctactctgtggatgagcgggtggactctgacagcaagtctgaggttgaagctctggctgaacaactgagtgaggaagaggaagaggaggaggaggaagaggaggaagaagaagaggaggaggaggaggaggaagaagaagaggaagatgaagagtctggcaatcagtctgacaggagtggttccagcggccgccgcaaagccaagaagaaatggcggaaggacagcccatgggtgaagccatcacggaaacggcggaaacgagagcctccgagggccaaggagccacgaggagtgaatggtgtgggctcctcaggccccagtgagtacatggaggtccctctggggtctctggagctgcccagcgaggggaccctctctcccaaccacgctggggtgtccaatgacacatcttcgctggagacagagcgcgggtttgaggagctgcccctctgcagctgccgcatggaggcccccaagatagaccgcatcagcgagagagcagggcacaagtgcatggccacagagagcgtggacggagagctgtccggctgcaatgctgccatcctcaagcgggagaccatgagaccgtccagccgtgtggccctgatggtgctctgtgagacccaccgtgcgcgcatggtcaaacatcactgctgccccggctgtggctacttctgtacggcgggcaccttcctggagtgtcaccccgacttccgtgtggcccaccgcttccacaaggcctgtgtgtcccagctgaacgggatggtcttctgtccccactgtggggaggacgcatctgaggcccaggaggtgaccatcccccggggggatggggtgaccccaccggctggcactgcagcacccgctcccccacccctggcccaggatgcccccgggagagcggacacttcccagcccagtgcccggatgcgggggcagggggagcctcggcgtccaccctgcgaccctctggccgacaccatcgacagctcagggccctccctaaccctgcccaatgggggctgcctctcagctgtagggctgccaccagggccaggccgggaggccctggagaaggccctggtcatccaggagtcagagaggcggaaaaagctccggttccaccctcggcagttgtacctgtccgtaaagcaaggggagctgcagaaggtgatcctgatgctgttggacaacctggaccccaacttccagagtgaccagcagagcaagcgcacacccctgcacgcggctgcccagaagggctctgtggagatctgccatgtgctgctgcaggctggagccaacatcaatgccgtggacaagcagcagcggacgccgctgatggaggccgtggtgaacaaccatctggaggtggctcgctacatggtgcagcgtgggggctgtgtctacagcaaggaggaagacggctccacctgcctccaccacgccgccaaaattgggaacctggagatggtcagcctgctgctgagcacgggacaggtggacgtcaatgcccaggacagcggcgggtggacccccatcatctgggctgcagagcacaagcacatagaagtgatccgcatgcttctgacacggggcgcggacgtcactctcaccgacaacgaggagaacatctgcctgcactgggcctccttcaccggcagcgccgccatcgccgaggtcctcctgaacgcccgctgcgacctccacgctgtcaattaccacggggacacacccctgcacatcgcggcccgggagagctaccacgactgcgtgctgttgttcctgtcacgcggggcgaaccctgagctgcggaacaaggagggggacacggcgtgggacctgacccccgagcggtccgacgtgtggtttgccctccagctcaaccgcaagctccggctcggggtggggaatcgggccatccgcacggagaagatcatctgccgggacgtggctcggggctatgagaacgtccccatcccctgtgtcaatggtgtggacggggagccctgccccgaggattacaagtacatctcagagaactgcgagacgtccaccatgaacattgaccgcaacattacccacctgcagcactgcacgtgtgtggatgactgctccagctccaactgcctgtgtggccagctcagcatccgctgttggtatgacaaggatgggcggctgctccaggaatttaacaagatcgagcccccgctgatttttgagtgtaaccaggcatgttcctgctggagaaactgcaagaacagagtagtgcagagcggcatcaaggtgcgactgcagctctaccgaacagccaagatgggctggggggtccgtgccctgcagaccattccccagggtaccttcatttgcgagtatgtcggagaactgatctctgatgccgaggctgatgtgagagaggacgattcttatctcttcgacttggacaacaaggatggagaggtgtactgcatcgatgcccgttactacggcaacatcagccgcttcatcaaccacttgtgtgaccccaacatcatccccgtccgagtcttcatgctgcaccaagacctgagatttccacgcatcgccttcttcagttcccgagacatccgggccggggaggagctggggtttgactatggtgaccgcttctgggacatcaaaagcaagtacttcacctgccagtgtggctctgagaagtgcaagcactcggctgaggccattgccctggagcagagccgcctggcccgcctggacccacaccctgagctgctgcctgagctcagctccctgccccccgtcaacccctgagatcggaccacagccactgttccacccctggacagccaccagctcagtagccacttctgccgcttgcccacagcctacgcctgggggcttttaccgtttcctctctcccttacttaccccttcacacattcctcagccagctcccagccaggccctggaggtctgacagcccctgtctcccagagctggttctttcctgggagggtgacttcagggctggccacccctccccccaagcgcctcctcccacccctgttccccaccctcaactgaagtttgatgaaattgagcctctgtgctaattggtttcctttgttctcaataaatgttgggtttataataaa

SEQ ID NO: 24 - protein, 1211 aa, Bos taurus
MAAAAGAAAAAAAEGEAPAEMGALVLEKEPRGATERVHGSLGDTSRSEETLPKANPDSLETAGPSSPASVTVTVGDEGADTPVGATPLIGDEPENLEGDGDLHGGRILLGHATKSFPSSPSKGGACPSRAKMSMTGAGKSPPSVQSLAMRLLSMPGAQGAPAAGPEPPPATASPEGQPKVHRARKTMSKPGNGQPPVPEKRPPEVQHFRMSDDVHSLGKVTSDVAKRRKLNSGGGLSEELASTRSSGEVTLEKGDPGSLEEWETVVGDDFSLYYDSYSVDERVDSDSKSEVEALAEQLSEEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLAQDAPGRADTSQPSARMRGQGEPRRPPCDPLADTIDSSGPSLTLPNGGCLSAVGLPPGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRAGEELGFDYGDRFWDIKSKYFTCQCGSEKCKHSAEAIALEQSRLARLDPHPELLPELSSLPPVNP

SEQ ID NO: 25 - DNA, 3947 bp, Mus musculus
  source 1..3947: mol_type = other DNA
agagatgcggggtctgccgagagggagggggctgatgcgggcccgggggcgggggcgtgcggcccccacgggcggccgcggccgcggtcgggggggcgcccaccgagggcgaggtaggccccgaagcctgctctcgctgcccagggcccaggcgtcttgggccccccagctgcctgccgggctgaccggccccccggttccttgtctcccctcccagggggaggcccccgctgagatgggggcgctgctgctggagaaggagccccgaggagccgccgagagagttcatagctctttgggggacacccctcagagtgaggagacccttcccaaggccaaccccgactccttggagcctgccggcccctcctctccggcctctgtcactgtcaccgtcggcgatgagggggctgacacccctgtcggggccgcatcactcatcggggacgaacccgagagcctggagggagatgggggtcgcatcgtgctgggccatgccacaaagtcgttcccctcttcccccagcaaggggggtgcctgtcccagtcgggccaaaatgtcaatgacaggggcaggaaagtcgcccccctcggtccagagtttggccatgaggctgttgagcatgcccggggcccagggagctgcaactgctgggcctgaaccctctccggcaacaactgccgcccaggaggggcagcccaaagtgcaccgagcccggaaaaccatgtccaaacctagcaacggacagcctccaatccctgagaagcggccccctgaagtccagcatttccgcatgagtgatgacatgcatctggggaaggtgacttcagatgtggccaaaaggaggaagctgaactctggtagcctgtccgaggacttgggctctgccgggggctcaggagatataatcctggagaagggagagcccaggcccctggaggagtgggagacggtggtgggcgatgacttcagcctgtactatgatgcgtactctgtggatgagcgggtggactctgacagcaagtctgaagtcgaagctctagctgaacagttgagtgaggaggaggaggaggaagaggaggaagaagaagaagaggaggaggaggaggaagaggaggaggaggaagaagaggacgaggagtcgggcaatcagtcagacaggagcggttctagtggccggcgcaaggccaagaagaaatggcggaaagacagcccgtgggtgaagccatctagaaaacggcggaaacgagagcctccgagggccaaggagccaagaggggtctccaatgacacgtcttcactggagacagaacgcgggtttgaggagctgcccctctgcagctgccgcatggaggctcccaagattgaccgcatcagcgagagagcagggcacaagtgcatggccacagagagtgtggatggagagctcctgggctgcaatgctgccatccttaagcgggagaccatgcggccgtctagccgcgtggcgctgatggtgctctgtgaggcccatcgagcccgcatggtcaagcaccattgctgcccgggctgcggctacttctgcacagcgggcaccttcctggaatgccaccccgactttcgtgtagctcaccgcttccataaggcctgcgtatcccagctcaatgggatggtcttctgtccccactgtggagaggatgcctcagaggcccaggaggtgaccattcctcggggcgatgggggaacacccccaattggcaccgcagctcctgctctgccacccctggcacatgatgccccagggcgagcggatacctcccagcctagcgcccgaatgcgagggcatggagagccgcggcgcccgccctgtgatcccctggctgacaccatcgacagctcagggccttcactgactctgcctaatgggggctgcctctccgctgtgggtctgcccccagggccgggcagggaagccctggaaaaagccttggtcatccaggagtctgagaggcggaagaagctgcgattccacccacggcagctgtacctgtcggtgaagcagggggagctgcagaaggtgatccttatgctgttagacaacctggaccccaacttccagagcgaccagcagagcaagcgcacgcccctgcacgcggccgcccagaaggggtcggtagagatctgtcatgtgctgctgcaggcaggagccaacatcaatgccgtagataagcaacaacgcacgccactaatggaggccgtggtgaacaaccacctggaggtggcacgctacatggtgcagttaggtggctgtgtctacagcaaggaagaggatggctccacctgtctacatcatgcagccaaaattgggaacttggaaatggtcagcctgctactgagcacaggacaggtggacgtcaatgcccaggacagtgggggctggacgcccatcatctgggcagccgagcacaagcacatcgatgtgattcgtatgctgctgacccggggtgccgatgtcaccctgactgacaatgaggaaaacatctgcctgcactgggcctccttcacgggtagtgccgccatcgctgaggtccttctgaatgcccagtgtgatctccatgctgtcaactaccatggggacacgcccctgcacatagccgccagggagagctaccatgactgtgttctgttgttcctgtctcgtggagccaaccctgagcttcggaacaaagaaggagacacggcatgggatctgaccccagagcgctctgatgtgtggtttgcactgcagctcaatcgaaagcttaggcttggggtagggaaccgggctgtccgcaccgagaagatcatctgccgggacgtagcccgaggctatgagaatgtacccatcccctgtgtcaatggtgtggatggggagccgtgcccggaggactacaagtacatctctgagaactgcgagacatcgaccatgaacatcgaccgcaacatcacccatctgcagcactgcacgtgtgtggatgactgctccagctccaattgcctatgtggtcagctcagtatccgatgctggtatgacaaggacgggcggctgctccaggagtttaacaagatcgagccccccctgatctttgagtgtaaccaggcatgctcctgctggagaagctgcaagaaccgcgtggtgcagagcggcatcaaggtacggctgcagctctaccggactgccaagatgggctggggggtccgagccttgcagaccatcccccagggcacgttcatctgcgagtatgtaggagagctgatctctgatgccgaggctgatgtgagagaggatgattcttacctcttcgatttagataacaaggatggcgaggtttactgcattgatgcccgttactatggcaacatcagccgattcattaaccacctgtgtgaccccaacatcatccctgtccgggttttcatgctgcaccaagatctacggttcccacgcattgccttcttcagctccagggacatccggactggggaggagctgggctttgactacggtgaccgattctgggacatcaagagcaagtatttcacctgccagtgtggctctgagaagtgcaagcattcagcggaggccatcgccctggagcagagccgcctggcccggctggacccccacccggagctgctccctgacctcagctccctgccccccatcaacacctgaggactcttaaaatccaggccgggcactgcccttcagacatttctccatcagagaccccagtaaggcctggaaggtcgatggcccctctcccagagctggtttctcactgggagtgcaagtgacttcagggctggccttccccactgagcctggcctcagttagctgattgaagttgggcctctgccagctgattttctgtgttctcaataaatgttgggtttggtaataaactgtctggtgattgtttcagga

SEQ ID NO: 26 - protein, 1229 aa, Mus musculus
MRGLPRGRGLMRARGRGRAAPTGGRGRGRGGAHRGRGRPRSLLSLPRAQASWAPQLPAGLTGPPVPCLPSQGEAPAEMGALLLEKEPRGAAERVHSSLGDTPQSEETLPKANPDSLEPAGPSSPASVTVTVGDEGADTPVGAASLIGDEPESLEGDGGRIVLGHATKSFPSSPSKGGACPSRAKMSMTGAGKSPPSVQSLAMRLLSMPGAQGAATAGPEPSPATTAAQEGQPKVHRARKTMSKPSNGQPPIPEKRPPEVQHFRMSDDMHLGKVTSDVAKRRKLNSGSLSEDLGSAGGSGDIILEKGEPRPLEEWETVVGDDFSLYYDAYSVDERVDSDSKSEVEALAEQLSEEEEEEEEEEEEEEEEEEEEEEEEEDEESGNQSDRSGSSGRRKAKKKWRKDSPWVKPSRKRRKREPPRAKEPRGVSNDTSSLETERGFEELPLCSCRMEAPKIDRISERAGHKCMATESVDGELLGCNAAILKRETMRPSSRVALMVLCEAHRARMVKHHCCPGCGYFCTAGTFLECHPDFRVAHRFHKACVSQLNGMVFCPHCGEDASEAQEVTIPRGDGGTPPIGTAAPALPPLAHDAPGRADTSQPSARMRGHGEPRRPPCDPLADTIDSSGPSLTLPNGGCLSAVGLPPGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQLGGCVYSKEEDGSTCLHHAAKIGNLEMVSLLLSTGQVDVNAQDSGGWTPIIWAAEHKHIDVIRMLLTRGADVTLTDNEENICLHWASFTGSAAIAEVLLNAQCDLHAVNYHGDTPLHIAARESYHDCVLLFLSRGANPELRNKEGDTAWDLTPERSDVWFALQLNRKLRLGVGNRAVRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRSCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPDLSSLPPINT

SEQ ID NO: 27 - DNA, 4049 bp, Mus musculus
  source 1..4049: mol_type = other DNA
agagatgcggggtctgccgagagggagggggctgatgcgggcccgggggcgggggcgtgcggcccccacgggcggccgcggccgcggtcgggggggcgcccaccgagggcgaggtaggccccgaagcctgctctcgctgcccagggcccaggcgtcttgggccccccagctgcctgccgggctgaccggccccccggttccttgtctcccctcccagggggaggcccccgctgagatgggggcgctgctgctggagaaggagccccgaggagccgccgagagagttcatagctctttgggggacacccctcagagtgaggagacccttcccaaggccaaccccgactccttggagcctgccggcccctcctctccggcctctgtcactgtcaccgtcggcgatgagggggctgacacccctgtcggggccgcatcactcatcggggacgaacccgagagcctggagggagatgggggtcgcatcgtgctgggccatgccacaaagtcgttcccctcttcccccagcaaggggggtgcctgtcccagtcgggccaaaatgtcaatgacaggggcaggaaagtcgcccccctcggtccagagtttggccatgaggctgttgagcatgcccggggcccagggagctgcaactgctgggcctgaaccctctccggcaacaactgccgcccaggaggggcagcccaaagtgcaccgagcccggaaaaccatgtccaaacctagcaacggacagcctccaatccctgagaagcggccccctgaagtccagcatttccgcatgagtgatgacatgcatctggggaaggtgacttcagatgtggccaaaaggaggaagctgaactctggtagcctgtccgaggacttgggctctgccgggggctcaggagatataatcctggagaagggagagcccaggcccctggaggagtgggagacggtggtgggcgatgacttcagcctgtactatgatgcgtactctgtggatgagcgggtggactctgacagcaagtctgaagtcgaagctctagctgaacagttgagtgaggaggaggaggaggaagaggaggaagaagaagaagaggaggaggaggaggaagaggaggaggaggaagaagaggacgaggagtcgggcaatcagtcagacaggagcggttctagtggccggcgcaaggccaagaagaaatggcggaaagacagcccgtgggtgaagccatctagaaaacggcggaaacgagagcctccgagggccaaggagccaagaggagtgaatggtgtgggttcctcagggcccagtgagtacatggaggttcctctggggtccctggagctgcccagcgaggggaccctctcccccaaccacgctggggtctccaatgacacgtcttcactggagacagaacgcgggtttgaggagctgcccctctgcagctgccgcatggaggctcccaagattgaccgcatcagcgagagagcagggcacaagtgcatggccacagagagtgtggatggagagctcctgggctgcaatgctgccatccttaagcgggagaccatgcggccgtctagccgcgtggcgctgatggtgctctgtgaggcccatcgagcccgcatggtcaagcaccattgctgcccgggctgcggctacttctgcacagcgggcaccttcctggaatgccaccccgactttcgtgtagctcaccgcttccataaggcctgcgtatcccagctcaatgggatggtcttctgtccccactgtggagaggatgcctcagaggcccaggaggtgaccattcctcggggcgatgggggaacacccccaattggcaccgcagctcctgctctgccacccctggcacatgatgccccagggcgagcggatacctcccagcctagcgcccgaatgcgagggcatggagagccgcggcgcccgccctgtgatcccctggctgacaccatcgacagctcagggccttcactgactctgcctaatgggggctgcctctccgctgtgggtctgcccccagggccgggcagggaagccctggaaaaagccttggtcatccaggagtctgagaggcggaagaagctgcgattccacccacggcagctgtacctgtcggtgaagcagggggagctgcagaaggtgatccttatgctgttagacaacctggaccccaacttccagagcgaccagcagagcaagcgcacgcccctgcacgcggccgcccagaaggggtcggtagagatctgtcatgtgctgctgcaggcaggagccaacatcaatgccgtagataagcaacaacgcacgccactaatggaggccgtggtgaacaaccacctggaggtggcacgctacatggtgcagttaggtggctgtgtctacagcaaggaagaggatggctccacctgtctacatcatgcagccaaaattgggaacttggaaatggtcagcctgctactgagcacaggacaggtggacgtcaatgcccaggacagtgggggctggacgcccatcatctgggcagccgagcacaagcacatcgatgtgattcgtatgctgctgacccggggtgccgatgtcaccctgactgacaatgaggaaaacatctgcctgcactgggcctccttcacgggtagtgccgccatcgctgaggtccttctgaatgcccagtgtgatctccatgctgtcaactaccatggggacacgcccctgcacatagccgccagggagagctaccatgactgtgttctgttgttcctgtctcgtggagccaaccctgagcttcggaacaaagaaggagacacggcatgggatctgaccccagagcgctctgatgtgtggtttgcactgcagctcaatcgaaagcttaggcttggggtagggaaccgggctgtccgcaccgagaagatcatctgccgggacgtagcccgaggctatgagaatgtacccatcccctgtgtcaatggtgtggatggggagccgtgcccggaggactacaagtacatctctgagaactgcgagacatcgaccatgaacatcgaccgcaacatcacccatctgcagcactgcacgtgtgtggatgactgctccagctccaattgcctatgtggtcagctcagtatccgatgctggtatgacaaggacgggcggctgctccaggagtttaacaagatcgagccccccctgatctttgagtgtaaccaggcatgctcctgctggagaagctgcaagaaccgcgtggtgcagagcggcatcaaggtacggctgcagctctaccggactgccaagatgggctggggggtccgagccttgcagaccatcccccagggcacgttcatctgcgagtatgtaggagagctgatctctgatgccgaggctgatgtgagagaggatgattcttacctcttcgatttagataacaaggatggcgaggtttactgcattgatgcccgttactatggcaacatcagccgattcattaaccacctgtgtgaccccaacatcatccctgtccgggttttcatgctgcaccaagatctacggttcccacgcattgccttcttcagctccagggacatccggactggggaggagctgggctttgactacggtgaccgattctgggacatcaagagcaagtatttcacctgccagtgtggctctgagaagtgcaagcattcagcggaggccatcgccctggagcagagccgcctggcccggctggacccccacccggagctgctccctgacctcagctccctgccccccatcaacacctgaggactcttaaaatccaggccgggcactgcccttcagacatttctccatcagagaccccagtaaggcctggaaggtcgatggcccctctcccagagctggtttctcactgggagtgcaagtgacttcagggctggccttccccactgagcctggcctcagttagctgattgaagttgggcctctgccagctgattttctgtgttctcaataaatgttgggtttggtaataaactgtctggtgattgtttcagga

SEQ ID NO: 28 - protein, 1263 aa, Mus musculus
MRGLPRGRGLMRARGRGRAAPTGGRGRGRGGAHRGRGRPRSLLSLPRAQASWAPQLPAGLTGPPVPCLPSQGEAPAEMGALLLEKEPRGAAERVHSSLGDTPQSEETLPKANPDSLEPAGPSSPASVTVTVGDEGADTPVGAASLIGDEPESLEGDGGRIVLGHATKSFPSSPSKGGACPSRAKMSMTGAGKSPPSVQSLAMRLLSMPGAQGAATAGPEPSPATTAAQEGQPKVHRARKTMSKPSNGQPPIPEKRPPEVQHFRMSDDMHLGKVTSDVAKRRKLNSGSLSEDLGSAGGSGDIILEKGEPRPLEEWETVVGDDFSLYYDAYSVDERVDSDSKSEVEALAEQLSEEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELLGCNAAILKRETMRPSSRVALMVLCEAHRARMVKHHCCPGCGYFCTAGTFLECHPDFRVAHRFHKACVSQLNGMVFCPHCGEDASEAQEVTIPRGDGGTPPIGTAAPALPPLAHDAPGRADTSQPSARMRGHGEPRRPPCDPLADTIDSSGPSLTLPNGGCLSAVGLPPGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQLGGCVYSKEEDGSTCLHHAAKIGNLEMVSLLLSTGQVDVNAQDSGGWTPIIWAAEHKHIDVIRMLLTRGADVTLTDNEENICLHWASFTGSAAIAEVLLNAQCDLHAVNYHGDTPLHIAARESYHDCVLLFLSRGANPELRNKEGDTAWDLTPERSDVWFALQLNRKLRLGVGNRAVRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRSCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPDLSSLPPINT

SEQ ID NO: 29 - DNA, 4023 bp, Rattus norvegicus
  source 1..4023: mol_type = other DNA
atgcggggtctgccgagagggagggggctgatgcgggcccgggggcgggggcgtgcggcccccacgggcggccgcggccggggtcgcgggggcgcccaccgtggacgaggtaggccccgaagcctgctctcgctgcccagggcccaggcgtcttgggccccccagctgcctgccgggctgaccggccctccggtcccttgtctcccctcccagggggaggcccccgctgagatgggggcgctgctgctggagaaggagccccgaggagccgccgagagagttcatggctctttgggggacacctctcatagtgaggagacccttcccaaggccaaccccgactccttggagcctaccggcccctcctctccggcctctgtcactgtcaccgtcggcgatgagggggctgacacccctgtcggggccacaccgctcatcggggaggaacccgagaacctggagggagatgggggtcgaatcctgctgggccatgccacaaagtcattcccctcttcccccagcaaggggggtgcctgtcccagtcgggccaaaatgtcaatgacaggggcaggaaagtcgcccccctcggtccagagtttggccatgaggctgttgagcatgcctggggcccagggagctgcaactgctgggcctgagccccctccggcaacaactgccggccaggaggggcagcccaaagtacaccgagctcggaaaaccatgtccaagcctagcaacggacagcctccagtccctgagaagcggccccctgaagtccagcatttccgcatgagtgacgacatgcatctgggaaaggtgacttcagatgtggccaaacggaggaagttgacctcgggcagcctgtcagaggacttgggctctgctgggggctcaggagaagtgatcctggagaagggagagcccaggcctctggaggagtgggagacggtggtgggcgatgacttcagcctgtactatgacgcgtactctgtggatgagcgggtggactctgacagcaagtctgaagtcgaagctctagctgaacagttgagtgaagaggaggaggaggaagaggaggaagaagaagaggaagaggaggaagaggaggaggaggaagaggaggaggaggatgaggagtcgggcaaccagtcagacaggagtggctctagcggccgacgcaaggccaagaagaaatggcggaaagacagcccatgggtgaagccatccaggaaacggcggaagcgagagcctccgagggccaaggagcccagaggagtgaatggtgtgggttcctcagggcccagtgagtacatggaggttcctctggggtccctggagctgcccagcgaggggaccctctcccccaaccacgctggggtctccaatgacacgtcttcgctggagacggagcgtgggtttgaggagctgcccctctgcagctgccgcatggaggcgcccaagattgaccgcatcagcgagagagcaggacacaagtgtatggccaccgaaagtgtggacggagagctgctgggttgcaatgccgccatcttgaagcgggagaccatgcggccgtctagccgtgtggcgctgatggtactgtgtgaggcccatcgagcccgcatggtcaagcaccattgctgcccaggctgtggctacttctgcacggcgggcaccttcctggagtgtcaccccgacttccgtgtagctcaccgcttccataaggcctgcgtgtcccagctcaatgggatggttttctgtccccactgtggggaggacgcctcagaggcccaggaggtgaccatccctcggggcgatgggggaacacccccggttggcactgtggcgcctgccccgccacccctggcacacgacgccccagggcgagcggatacctcccagcccagtgcccgaatgcgaggacacggagagccaaggcgcccaccctgtgatcccctggccgacaccattgacagctcggggccttctctgactctgcctaatggcggctgcctctcagctgtgggtctgcccccagggccaggccgggaagccctggaaaaagccttggtcatccaggagtccgagaggaggaagaagctgcggttccacccgcggcagctctacctgtcggtgaagcagggggagctgcagaaggtgatcctcatgctgctcgacaacctggaccccaacttccagagcgaccagcagagcaagcgcacgcccctgcacgcagccgcccagaaggggtccgtggagatctgtcacgtgctgctacaggcaggagccaacatcaacgccgtggataagcagcagcgcaccccgctgatggaggccgtggtgaacaaccacctggaggtggctcgctacatggtgcagctgggcggctgtgtgtacagcaaggaagaggatggctctacctgtctccatcatgcggccaaaattgggaacttggaaatggtcagcctgctgctgagcacagggcaggtggacgtcaatgcccaggacagtgggggctggacacccatcatctgggcagccgagcacaagcacatcgacgtgatccgtatgctgctgacccggggcgccgacgtcacccttaccgacaacgaggaaaacatctgcctgcactgggcttccttcacgggtagtgccgctattgccgaggtcctcctgaatgcccagtgcgacctccatgctgtcaactaccacggggacacgcccctgcacatagccgccagggagagctaccatgactgcgttctgttgttcctgtcccgtggagccaaccctgagcttcggaacaaagaaggggacacggcgtgggatctgaccccagagcgctccgatgtgtggtttgcactgcagctcaatcgcaagcttaggctcggggtagggaaccgggctgtccgcaccgagaagatcatctgccgggatgtagcccgaggctatgagaatgtgcccattccctgtgtcaatggcgtggatggggagccatgcccagaggactataagtacatctccgagaactgtgaaacgtccaccatgaacatcgaccgaaacatcactcacctgcagcactgcacgtgtgttgacgactgctccagctccaactgcctgtgtggccagctaagcatccgatgctggtatgataaggacgggcggctgctccaggagtttaacaagatcgagccccccctgatctttgagtgtaaccaggcatgctcctgctggagaagctgcaagaaccgcgtggtgcagagcggcatcaaggtgcggctgcagctctaccggacggccaagatgggctggggggtccgtgccttgcagaccattccccagggcacgttcatctgcgagtacgtcggggagctgatctccgacgccgaggcagacgtgagagaggatgattcttacctcttcgacttagacaacaaggatggtgaggtctactgcattgatgcccgttactatggcaacatcagccgcttcattaaccacctgtgcgaccccaacatcatccctgttcgggtcttcatgctgcatcaagacctacggttccctcgcatcgccttcttcagctccagggacatccggaccggggaggagctggggtttgactacggcgaccggttctgggatatcaagagcaagtatttcacctgccagtgtggctctgagaagtgcaagcattcagctgaggccatcgccctggagcagagccgcctggcccgtctggacccccacccggagctgctccctgacctcagctccctgccccccatcaacacctgaggactgttaaaatccaggccggggcactgcccctcagacattcccccatcggaggaccccagtaaggcctggaaggtttacagcccctctcccagagctggtttctcactgggagtgagtgacttcagggctggcctaccccactgagcctggcctcagttcgctcattgaagttgggcctctgccaactgattttctgtgctctcaataaatgttgggtttggtaataaa

SEQ ID NO: 30 - protein, 1263 aa, Rattus norvegicus
MRGLPRGRGLMRARGRGRAAPTGGRGRGRGGAHRGRGRPRSLLSLPRAQASWAPQLPAGLTGPPVPCLPSQGEAPAEMGALLLEKEPRGAAERVHGSLGDTSHSEETLPKANPDSLEPTGPSSPASVTVTVGDEGADTPVGATPLIGEEPENLEGDGGRILLGHATKSFPSSPSKGGACPSRAKMSMTGAGKSPPSVQSLAMRLLSMPGAQGAATAGPEPPPATTAGQEGQPKVHRARKTMSKPSNGQPPVPEKRPPEVQHFRMSDDMHLGKVTSDVAKRRKLTSGSLSEDLGSAGGSGEVILEKGEPRPLEEWETVVGDDFSLYYDAYSVDERVDSDSKSEVEALAEQLSEEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELLGCNAAILKRETMRPSSRVALMVLCEAHRARMVKHHCCPGCGYFCTAGTFLECHPDFRVAHRFHKACVSQLNGMVFCPHCGEDASEAQEVTIPRGDGGTPPVGTVAPAPPPLAHDAPGRADTSQPSARMRGHGEPRRPPCDPLADTIDSSGPSLTLPNGGCLSAVGLPPGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQLGGCVYSKEEDGSTCLHHAAKIGNLEMVSLLLSTGQVDVNAQDSGGWTPIIWAAEHKHIDVIRMLLTRGADVTLTDNEENICLHWASFTGSAAIAEVLLNAQCDLHAVNYHGDTPLHIAARESYHDCVLLFLSRGANPELRNKEGDTAWDLTPERSDVWFALQLNRKLRLGVGNRAVRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRSCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPDLSSLPPINT

SEQ ID NO: 31 - DNA, 4153 bp, Equus caballus
  source 1..4153: mol_type = other DNA
ttctcggcccctgagatgcggggtctaccgagagggagggggttgatgcgggcccgggggaggggtcgtgcggcccctccgggcagccgaggccgcggaagggggggcccccatagaggaagaggtaggccccggagcctactctctcttcccagggcccaggcgtcctgggccccccaacttcctactgggctgaccagtcctcctatcccttgtgtcccctcccagggggaggcccctgctgagatgggggcgctggtgctggagaaggagcccagaggagccaccgagagagttcatggctctttgggggacacccctcgtagtgaggagactctgcccaaggccaaccctgactccctggagcctgctggcccctcatccccagcctctgtcaccgtgaccgtcggcgatgagggggctgacacccctgtaggggccacaccacttatcggggaagaacctgagaatctcgagggagatggggacctccatgggggccgcatcctgctgggccatgccacaaagtcgttcccgtcttcccccagcaaggggagtgcctgtcccagccgggccaagatgtcaatgacaggggctggaaaatcacccccatcagtccaaagtttggctatgaggctgctgagtatgccaggggctcagggggcagtggcagcaggacctgaaccccctcctgctacagccggcccagaggggccacccaaggtccaccgagccaggaaaaccatgtccaaacccggcaacggacagcccccagtccctgagaagcggccccctgaagtgcagcatttccgcatgagtgatgacgtgcactccctggggaaggtcacctcagatgtggccaagaggaggaagctgaacgcaggaggtggcctgtcagaggagttgggctctgcccggggttcgggagaagtgaccctggagaagggagaccctgggtccctggaggagtgggagacagtagtgggtgacgacttcagcctctactatgattcctactctgtagatgagcgtgtggactctgacagcaagtctgaggttgaagctctcgctgaacaactgagcgaggaagaggaggaggaggaagaggaagaagaggaagaagaagaggaagaggaggaagaagaggaagaagaagaagatgaggagtcaggcaatcagtcagacagaagtggttccagcggccggcgcaaagccaagaagaaatggcggaaggacagcccctgggtgaagccatcacggaaacggcggaagcgggagcctccgagggccaaggagccacgaggagtgaatggtgtgggctcctcaggccccagtgagtacatggaggtccctctggggtccctggagctgcccagcgaggggaccctctcccccaaccacgctggggtgtccaatgacacgtcttcactggagactgagcgcgggtttgaggagttgcccctctgcagctgccgcatggaggcgcccaagattgaccgcatcagcgagagagcggggcacaagtgcatggccacggagagtgtggatggagagctgtcaggctgcaatgctgcaatcctcaagcgggagaccatgaggccatcaagccgagtggcactgatggtgctctgtgagacccaccgcgcccgcatggtcaaacaccactgctgcccgggctgtggctacttctgtacagcgggcaccttcctggagtgccaccccgacttccgtgtggcccaccgcttccacaaggcctgtgtgtcccagctgaatgggatggttttctgtcctcactgtggggaggacgcatctgaagcccaggaggtgaccatcccccggggagacggggtgaccccaccagccggcactgccgcccctgcccccccacccctggcccaggatgcccctggaagagcagacacttcccagcccagcgcccggatgcgagggcatggggagccccggcgcccaccctgtgaccctctggctgacaccatcgacagctcagggccctccctgaccctgcccaatgggggctgcctctcagccgtggggctgccacctgggccaggccgggaggccctggagaaggccctggtcatccaggagtcagagaggcggaagaaactccgtttccacccccggcagttgtacctgtcagtgaagcagggggagctgcagaaggtgatcctcatgctgttggacaacctggaccccaacttccagagcgaccagcagagcaagcgcacgcctctgcatgcagccgcccagaagggctccgtggagatctgccacgtgctgctgcaggctggagccaatatcaatgcagtggacaagcagcagcggacgccgctgatggaggccgtggtgaacaaccacctggaggttgcacgctacatggtacagcgcggtggctgtgtctacagcaaggaggaagacggttccacctgtctccaccacgcagccaaaattgggaacttggagatggtcagcctgctgctcagcacaggacaggtggacgtcaatgcccaggacagtggggggtggacacccatcatctgggccgcagagcacaagcacatcgaggtgatccgcatgctgctgacacggggcgctgacgtcaccctcacggacaacgaggaaaacatctgcctgcactgggcttccttcaccggcagtgccgccatcgccgaggtgctactgaacgcccgctgcgacctccatgctgtgaactaccatggggacacgcccctgcacatcgcagcccgggagagctaccacgactgcgtgctgttgttcctgtcacgtggggcaaaccctgaacttcggaacaaggagggggacacagcatgggacctgactcctgagcgctctgacgtgtggtttgcactccagctcaaccgcaagcttcggctcggggtgggaaatcgggccatccgcacggagaggatcatctgccgggatgtggctcggggctacgagaatgtgcccattccctgtgtcaacggcgtggacggggagccctgccccgaggattacaagtacatctcagagaactgcgagacgtccaccatgaacatcgaccgcaacatcacccacctgcagcactgcacgtgtgtggacgactgctccagctccaactgcctgtgtggccagctcagcatccgctgctggtatgataaggacgggcggctgctccaggaatttaacaagatcgagccgccgctgatttttgagtgtaaccaggcgtgctcctgctggaggaactgcaagaaccgcgtagtgcagagcggcatcaaggtgcgactgcagctctaccgaacagccaagatgggctggggggtgcgcgccctgcagaccatcccccaggggaccttcatctgcgagtatgtcggggagctgatctccgatgctgaggccgatgtgagagaggatgattcttatctcttcgacttagacaacaaggatggagaggtgtactgcattgatgctcgttactatggcaacatcagccgcttcatcaaccacctgtgtgaccccaacatcatccccgtccgggtcttcatgctgcaccaagacctgcgatttccacgcattgccttcttcagttcccgagacatccggactggggaggagctggggtttgactatggtgaccgcttctgggacatcaaaagcaaatatttcacctgccagtgtggctctgagaagtgcaagcactcagccgaggccatcgccctggagcagagccgcctggcccgcctggacccccaccctgagctgctgcctgagctcagctccctgccccccgtcaacccctgagagcggaccacaccctcccctccccacccctggatggccaccggctcagcagccgcctctgccacttgctgctcacagcccacacctggggatgctgccatcttctctccccctaccccttcacacattcctcggccagagaccccagccaggccctggaggtctgacagcccctctctcccagagctggttcctccctgggagagtgacttcagggctggccaccccgacccgtacccccattccccattgctgaagattgatgaattgaaatgggcctctatgccaactggtttcctttgttctcaataaatgttgggtttggtaataa

SEQ ID NO: 32 - protein, 1268 aa, Equus caballus
MRGLPRGRGLMRARGRGRAAPPGSRGRGRGGPHRGRGRPRSLLSLPRAQASWAPQLPTGLTSPPIPCVPSQGEAPAEMGALVLEKEPRGATERVHGSLGDTPRSEETLPKANPDSLEPAGPSSPASVTVTVGDEGADTPVGATPLIGEEPENLEGDGDLHGGRILLGHATKSFPSSPSKGSACPSRAKMSMTGAGKSPPSVQSLAMRLLSMPGAQGAVAAGPEPPPATAGPEGPPKVHRARKTMSKPGNGQPPVPEKRPPEVQHFRMSDDVHSLGKVTSDVAKRRKLNAGGGLSEELGSARGSGEVTLEKGDPGSLEEWETVVGDDFSLYYDSYSVDERVDSDSKSEVEALAEQLSEEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLAQDAPGRADTSQPSARMRGHGEPRRPPCDPLADTIDSSGPSLTLPNGGCLSAVGLPPGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTERIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELSSLPPVNP